(19) United States
(12) Patent Application Publication
(10) Pub. No.: US 2012/0009222 A1
(43) Pub. Date: Jan. 12, 2012

(54) MODULATION OF THE IMMUNE RESPONSE

(75) Inventors: David-Huy Nhu Nguyen, Mountain View, CA (US); Daniel Griffith Anderson, Sudbury, MA (US); Robert S. Langer, Newton, MA (US); Kerry Peter Mahon, Cambridge, MA (US)

(73) Assignee: Massachusetts Institute of Technology, Cambridge, MA (US)

(21) Appl. No.: 13/126,260

(22) PCT Filed: Oct. 27, 2009
(86) PCT No.: PCT/US2009/005810

§ 371 Date: Sep. 16, 2011

Related U.S. Application Data
(60) Provisional application No. 61/108,601, filed on Oct. 27, 2008.

(51) Int. Cl. A61K 31/7088; A61P 37/02; B82Y 5/00

(52) U.S. Cl. 424/283.1; 977/773; 977/795; 977/918

(57) ABSTRACT

The present invention provides lipidoids that can be used to modulate the immune response in a subject. Lipidoids are prepared by the conjugate addition of an amine to an acrylate to acrylamide. The lipidoids form complexes or particles with an immunostimulatory polynucleotide, which are then administerd to a subject. Such compositions have been found to stimulate the production of cytokines and increase both humoral and cell-mediate immune response. The invention also provides pharmaceuti-cal compositions thereof and methods for using the same.

### RELATED APPLICATIONS

The present application claims priority under 35 U.S.C. §119(e) to U.S. provisional application, U.S. Ser. No. 61/108,601, filed Oct. 27, 2008, which is incorporated herein by reference.

### GOVERNMENT SUPPORT

This invention was made with U.S. Government support under contract number EB000244 awarded by National Institutes of Health. The U.S. Government has certain rights in the invention.

### BACKGROUND OF THE INVENTION

The treatment of human diseases through the application of nucleic acid-based drugs such as DNA and RNA has the potential to revolutionize the medical field (Anderson Nature 392 (Suppl.):25-30, 1996; Friedman Nature Med. 2:144-147, 1996; Crystal Science 270:404-410, 1995; Mulligan Science 260:926-932, 1993; each of which is incorporated herein by reference). Thus far, the use of modified viruses as gene transfer vectors has generally represented the most clinically successful approach to gene therapy. While viral vectors are currently the most efficient gene transfer agents, concerns surrounding the overall safety of viral vectors, which includes the risk of undesired immune responses, have resulted in efforts to develop non-viral alternatives (for leading references, see: Luo et al. Nat. Biotechnol. 18:33-37, 2000; Behr Acc. Chem. Res. 26:274-278, 1993; each of which is incorporated herein by reference). Current alternatives to viral vectors include polymeric delivery systems (Zauner et al. Adv. Drug Del. Rev. 30:97-113, 1998; Kabanov et al. Bioconjugate Chem. 6:7-20, 1995; each of which is incorporated herein by reference), liposomal formulations (Miller Angew. Chem. Int. Ed. 37:1768-1785, 1998; Hope et al. Molecular Membrane Technology 15:1-14, 1998; Deshmukh et al. New J. Chem. 21:113-124, 1997; each of which is incorporated herein by reference), and “naked” DNA injection protocols (Sanford Trends Biotechnol. 6:288-302, 1988; incorporated herein by reference). While these strategies have yet to achieve the clinical effectiveness of viral vectors, the potential safety, processing, and economic benefits offered by these methods (Anderson Nature 392 (Suppl.):25-30, 1996; incorporated herein by reference) have ignited interest in the continued development of non-viral approaches to gene therapy (Boussif et al. Proc. Natl. Acad. Sci. USA 92:7297-7301, 1995; Putnam et al. Macromolecules 32:3658-3662, 1999; Lim et al. J. Am. Chem. Soc. 121:5633-5639, 1999; Gonzalez et al. Bioconjugate Chem. 10:1068-1074, 1999; Kukowska-Latallo et al. Proc. Natl. Acad. Sci. USA 93:4897-4902, 1996; Tang et al. Bioconjugate Chem. 7:703-714, 1996; Haensler et al. Bioconjugate Chem. 4:372-379, 1993; each of which is incorporated herein by reference).

Innate immune activation is a crucial step in activating mammalian responses to microbial infection ultimately leading to protective adaptive immunity. Activation of pattern recognition receptors (PRRs) allows for rapid identification of common pathogen-associated molecular patterns (PAMPs) without the need for prior education of an adaptive response.^{1, 2} The immune system has evolved methods for recognizing pathogens through a variety of receptors including the toll-like receptors (TLRs) (Cristofaro et al., Drugs (2006) 66: 15-29; Akira et al., C R Biol (2004) 327: 581-589; Hornung et al., J. Immunol. (2002) 168: 4531-4537; each of which is incorporated herein by reference). The Toll-like receptors (TLR), of which eleven have been identified in humans, recognize conserved structures among a diverse group of pathogens such as long dsRNA (TLR3), lipopolysachharide of bacterial cell walls (TLR4), and flagella (TLR5).² Long dsRNA interacts with TLR3 to induce interferon responses in various cell types (Marques et al., Nat Biotechnol (2005) 23: 1399-1405, incorporated herein by reference). Nucleic acids can be recognized by TLRs 7, 8, and 9, which comprise a closely related genetic sub-family whose expression is species-dependent and cell-type specific, and is functionally compartmentalized to the endosome.³ siRNAs have been shown to interact with TLR3, TLR7, and TLR8 to induce interferon responses (Kleinman et al., Nature (2008) 452: 591-597; Hornung et al., Nat. Med. (2005) 11: 263-270; Sioud et al., J Mol Biol (2005) 348: 1079-1090; each of which is incorporated herein by reference). These small siRNAs that generate immune responses through interferon-alpha and other cytokines are called immunostimulatory RNA (is RNA). TLR9 recognizes CpG sequences in unmethylated bacterial or viral DNA and synthetic CpG oligodeoxynucleotides (ODN). The activation of TLRs 7 and 8 is a hallmark of innate immune activation by RNA viruses. In humans, TLR7 seems to be highly expressed and functional mainly in plasmacytoid dendritic cells (pDCs) and B-cells, while TLR8 expression is localized mostly to monocytes, myeloid DCs (mDCs), and monocyte-derived DCs (moDCs).⁴

In particular, recognition of bacterial and viral nucleic acids in the endosomal compartment of plasmacytoid dendritic cells (TLR7 and TLR9) and myeloid dendritic cells (TLR3 and TLR8) results in a characteristic type I interferon response and coordinated Th1-biasing cytokine profile.¹ This innate immune response can have clinically relevant effects by activating anti-viral defenses² and increasing immune surveillance of cancer.⁵⁻⁶ Vaccine adjuvants can function by increasing coupling of innate and adaptive responses or by directing sustained availability of antigen to specific cells in the lymph nodes.⁷ Activation of the innate immune system is a critical step in generation of the second signal, or “danger signal,” necessary for efficient production of specific T-cell mediated responses and class switching to high-affinity antibodies.^{1,8} Thus, TLR activation may be useful for adjuvanting immune responses to vaccines.

Small-molecule agonists of TLR7 and TLR8 such as imiquimod and R-848 have been in used clinical as cancer therapies.^{5, 9} However, therapeutic activation of TLR7 and TLR8 with small single-stranded RNAs, the natural ligands for TLR7/8,^{10,11} has proven difficult due to low stability, nuclease degradation,¹²⁻¹⁴ and the requirement of endosomal uptake.^{10, 15} Many groups have focused on strategies to chemically or physically alter ssRNA and siRNA to increase serum stability and increase circulation time, but these modifications may also inhibit TLR activation^{14, 16-18} or require impractically large dosing¹² to achieve immunostimulatory effects.

There exists a continuing need for non-toxic, biodegradable, biocompatible lipid-like compounds that can be used to transfect polynucleotides and that can be prepared efficiently and economically. Such compounds may be used to modulate an immune response in a subject.

### SUMMARY OF THE INVENTION

The present invention stems from the recognition that lipidoids complexed with polynucleotides (particularly, is RNA, ssRNA, dsRNA, CpG oligonucleotides, CpG oligodeoxynucleotides, unmethylated bacterial or viral DNA) are useful for modulating the immune system of a subject. The present invention provides methods for modulating an immune response in a subject by administering a composition (e.g., a particle) comprising an immunostimulatory polynucleotide and a lipidoid. The design criteria for delivering is RNA for immune modulation are different from those of DNA delivery and siRNA delivery. For example, the is RNA typically needs to be delivered to the endosome where the TLRs are found rather than the cytosol or nucleus. Such compositions of an immunostimulatory polynucleotide and a lipidoid have been found useful in stimulating the production of cytokines and/or increasing both humoral and cell-mediated immune responses. Surprisingly, the lipidoids themselves have been found to have both RNA-specific and non-specific adjuvant activity. The invention also provides novel lipidoids useful in modulating immune function, as well as compositions of lipidoids. Thus, the present invention represents an important advance in the field of nucleic acid delivery systems and immune modulation.

In one aspect, the present invention provides novel lipidoids. Lipidoids have previously been described in detail in Akinc et al., Nat Biotechnol (2008) 26: 561-569; U.S. patent application U.S. Ser. No. 11/453,222, filed Jun. 14, 2006, and international PCT patent application PCT/US06/23171, publication number WO/2006/138380, filed Jun. 14, 2006, which claim priority to U.S. provisional application 60/785,176, filed Mar. 23, 2006, and U.S. provisional application 60/690,608, filed Jun. 15, 2005, all of which are incorporated herein by reference. The lipidoids may be synthesized by reacting an amine with an acrylate or acrylamide containing an aliphatic tail.

In certain embodiments, lipidoids of the invention are of the formula:

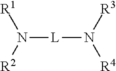

wherein L is a substituted or unsubstituted alkylene group having between one and six carbon atoms, inclusive;

R¹, R², R³, and R⁴ are independently selected from the group consisting of hydrogen, C₁-C₆ alkyl, C₃-C₇ cycloalkyl, —(CH₂)ₙOR^{A}, —(CH₂)ₙN(R^{A})₂, Y, —(CH₂)ₙN(Y)₂, —(CH₂)ₙNR^{A}Y; wherein each occurrence of R^{A} is independently a hydrogen; a protecting group; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted aryl; substituted or unsubstituted heteroaryl; alkoxy; aryloxy; alkylthio; arylthio; amino; alkylamino; dialkylamino; heteroaryloxy; or heteroarylthio moiety; with the proviso that at least one of R¹, R², R³, and R⁴ must equal Y, —(CH₂)ₙN(Y)₂, or —(CH₂)ₙNR^{A}Y;

R¹ and R² may be taken together with the intervening atoms to form a cyclic structure;

R³ and R⁴ may be taken together with the intervening atoms to form a cyclic structure; each occurrence of Y is independently selected from the group consisting of —CH₂CH₂CO₂R⁸ or —CH₂CH₂CONHR⁸; wherein each occurrence of R⁸ is independently an alkyl chain of 6-30 carbon atoms, inclusive;

each occurrence of n is independently an integer between one and six, inclusive; and salts thereof.

In certain embodiments, each instance of Y in the compounds described herein is independently selected from the group consisting of:

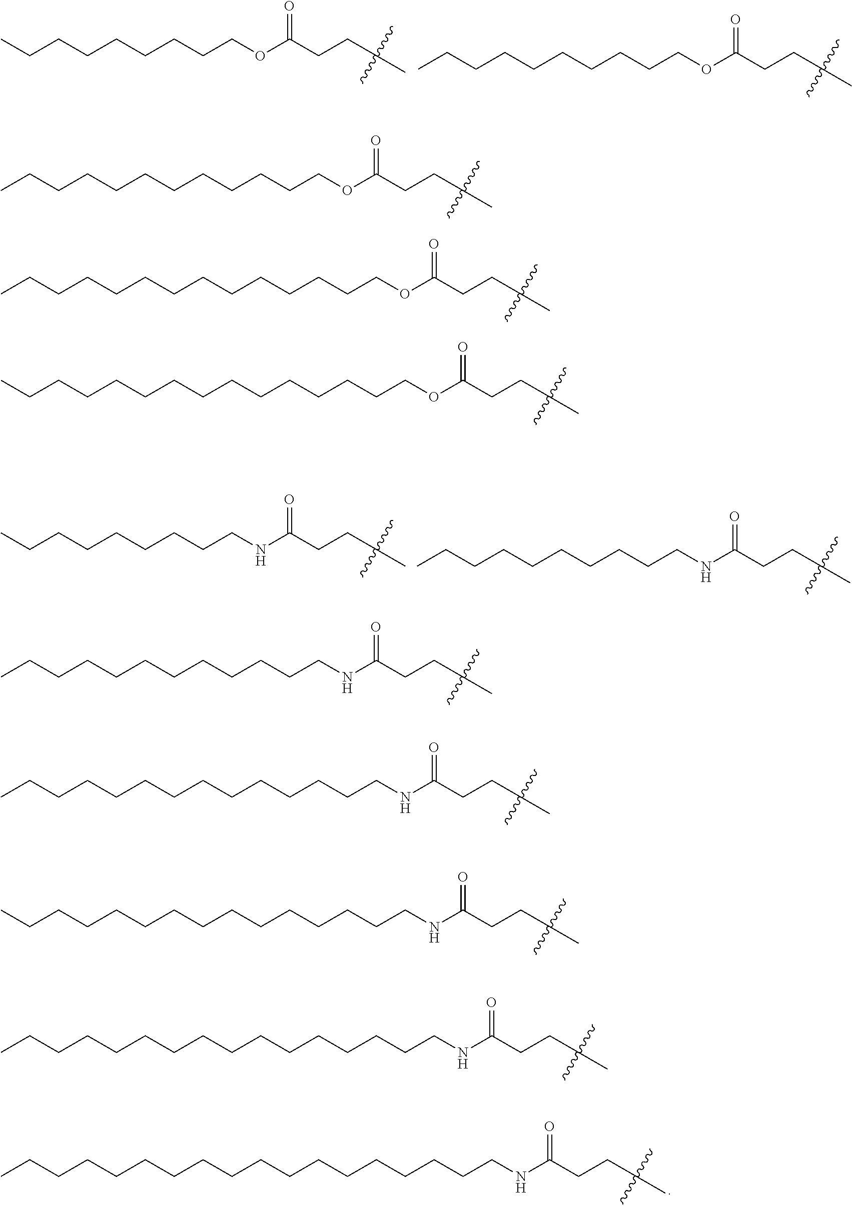

Lipidoids may be protonated or alkylated to form quaternary amines with a permanent positive charge. All or a portion of the amines may be protonated or alkylated. For example, a secondary or tertiary amine may be alkylated or acylated to for a quaternary amine. See, e.g., FIG. 16.

In certain embodiments, lipidoids of the current invention are of the formula:

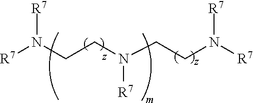

wherein

each occurrence of z is an integer between 1 and 10, inclusive;

m is an integer between 0 and 10, inclusive;

each occurrence of R₇ is hydrogen or Y; and

each occurrence of Y is independently selected from the group consisting of —CH₂CH₂CO₂R⁸ or —CH₂CH₂CONHR⁸; wherein each occurrence of R⁸ is independently an alkyl chain of 6-30 carbon atoms, inclusive; and salts thereof. In certain embodiments, the lipidoid is of the formula:

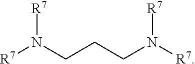

In certain embodiments, the lipidoid is of the formula:

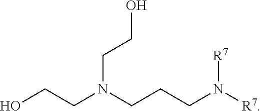

In certain embodiments, the lipidoid is of the formula:

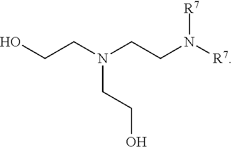

The lipidoids described herein are useful in the delivery of nucleic acids to cells. It is thought that lipidoids associate with nucleic acids. The lipidoids described herein may be particularly useful in delivering immunostimulatory polynucleotides, such as is RNA, ssRNA, or CpG DNA.

In another aspect, the invention provides compositions of lipidoids. A composition may contain lipidoids with the same amine core but different numbers of tails. A composition may contain lipidoids with a core amine and different tails, that is, the chemical identity of the tails is different. In certain embodiments, the composition further comprises an immunostimulatory polynucleotide. In certain embodiments, the composition further comprises a polymer. The compositions may be useful in delivering immunostimulatory polynucleotides to a subject. The compositions may be administered to a subject (e.g., human, mouse, rat, dog, cat) subcutaneously or intramuscularly. A therapeutically effective amount of the composition is administered to a subject to stimulate a desired immune response in the subject. The invention also provides kits for use in the present invention. The kit may include multiple doses of the lipidoid/immunostimulatory polynucleotide composition.

### DEFINITIONS

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito: 1999, the entire contents of which are incorporated herein by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

One of ordinary skill in the art will appreciate that the synthetic methods, as described herein, utilize a variety of protecting groups. By the term “protecting group”, as used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group should be selectively removable in good yield by readily available, preferably non-toxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen, and carbon protecting groups may be utilized. Hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS),1,3-(1,1,3,3-tetraisopropyldisiloxanylidene)derivative (TIPDS), tetra-t-butoxydisiloxane-1,3-diylidene derivative (TBDS), cyclic carbonates, cyclic boronates, ethyl boronate, and phenyl boronate. Amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fern), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilyethanesulfonamide (SES),9-anthracenesulfonamide, 4-(4′,8′-dimethoxynaphthylmethyl)benzenesulfonamide (DNMBS), benzylsulfonamide, trifluoromethylsulfonamide, and phenacylsulfonamide. Exemplary protecting groups are detailed herein, however, it will be appreciated that the present invention is not intended to be limited to these protecting groups; rather, a variety of additional equivalent protecting groups can be readily identified using the above criteria and utilized in the method of the present invention. Additionally, a variety of protecting groups are described in Protective Groups in Organic Synthesis, Third Ed. Greene, T. W. and Wuts, P. G., Eds., John Wiley & Sons, New York: 1999, the entire contents of which are hereby incorporated by reference.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of infectious diseases or proliferative disorders. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term “aliphatic”, as used herein, includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl”, “alkynyl”, and the like. Furthermore, as used herein, the terms “alkyl”, “alkenyl”, “alkynyl”, and the like encompass both substituted and unsubstituted groups. In certain embodiments, as used herein, “lower alkyl” is used to indicate those alkyl groups (cyclic, acyclic, substituted, unsubstituted, branched or unbranched) having 1-6 carbon atoms.

In certain embodiments, the alkyl, alkenyl, and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms. Illustrative aliphatic groups thus include, but are not limited to, for example, methyl, ethyl, n-propyl, isopropyl, cyclopropyl, —CH₂-cyclopropyl, vinyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl, n-hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl, and the like.

The term “alkyl” as used herein refers to saturated, straight- or branched-chain hydrocarbon radicals derived from a hydrocarbon moiety containing between one and twenty carbon atoms by removal of a single hydrogen atom. Examples of alkyl radicals include, but are not limited to, methyl, ethyl, propyl, isopropyl, n-butyl, tert-butyl, n-pentyl, neopentyl, n-hexyl, n-heptyl, n-octyl, n-decyl, n-undecyl, and dodecyl.

The term “alkenyl” denotes a monovalent group derived from a hydrocarbon moiety having at least one carbon-carbon double bond by the removal of a single hydrogen atom. Alkenyl groups include, for example, ethenyl, propenyl, butenyl, 1-methyl-2-buten-1-yl, and the like.

The term “alkynyl” as used herein refers to a monovalent group derived form a hydrocarbon having at least one carbon-carbon triple bond by the removal of a single hydrogen atom. Representative alkynyl groups include ethynyl, 2-propynyl (propargyl), 1-propynyl, and the like.

The term “alkoxy”, or “thioalkyl” as used herein refers to an alkyl group, as previously defined, attached to the parent molecule through an oxygen atom or through a sulfur atom. In certain embodiments, the alkyl, alkenyl, and alkynyl groups contain 1-20 alipahtic carbon atoms. In certain other embodiments, the alkyl, alkenyl, and alkynyl groups contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups contain 1-6 aliphatic carbon atoms. In yet other embodiments, the alkyl, alkenyl, and alkynyl groups contain 1-4 aliphatic carbon atoms. Examples of alkoxy, include but are not limited to, methoxy, ethoxy, propoxy, isopropoxy, n-butoxy, tert-butoxy, neopentoxy, and n-hexoxy. Examples of thioalkyl include, but are not limited to, methylthio, ethylthio, propylthio, isopropylthio, n-butylthio, and the like.

The term “alkylamino” refers to a group having the structure —NHR′, wherein R′ is aliphatic, as defined herein. In certain embodiments, the aliphatic group contains 1-20 aliphatic carbon atoms. In certain other embodiments, the aliphatic group contains 1-10 aliphatic carbon atoms. In yet other embodiments, the aliphatic group employed in the invention contain 1-8 aliphatic carbon atoms. In still other embodiments, the aliphatic group contains 1-6 aliphatic carbon atoms. In yet other embodiments, the aliphatic group contains 1-4 aliphatic carbon atoms. Examples of alkylamino groups include, but are not limited to, methylamino, ethylamino, n-propylamino, iso-propylamino, cyclopropylamino, n-butylamino, tert-butylamino, neopentylamino, n-pentylamino, hexylamino, cyclohexylamino, and the like.

The term “carboxylic acid” as used herein refers to a group of formula —CO₂H.

The term “dialkylamino” refers to a group having the structure —NRR′, wherein R and R′ are each an aliphatic group, as defined herein. R and R′ may be the same or different in an dialkyamino moiety. In certain embodiments, the aliphatic groups contains 1-20 aliphatic carbon atoms. In certain other embodiments, the aliphatic groups contains 1-10 aliphatic carbon atoms. In yet other embodiments, the aliphatic groups employed in the invention contain 1-8 aliphatic carbon atoms. In still other embodiments, the aliphatic groups contains 1-6 aliphatic carbon atoms. In yet other embodiments, the aliphatic groups contains 1-4 aliphatic carbon atoms. Examples of dialkylamino groups include, but are not limited to, dimethylamino, methyl ethylamino, diethylamino, methylpropylamino, di(n-propyl)amino, di(iso-propyl)amino, di(cyclopropyl)amino, di(n-butyl)amino, di(tert-butyl)amino, di(neopentyl)amino, di(n-pentyl)amino, di(hexyl)amino, di(cyclohexyl)amino, and the like. In certain embodiments, R and R′ are linked to form a cyclic structure. The resulting cyclic structure may be aromatic or non-aromatic. Examples of cyclic diaminoalkyl groups include, but are not limited to, aziridinyl, pyrrolidinyl, piperidinyl, morpholinyl, pyrrolyl, imidazolyl, 1,3,4-trianolyl, and tetrazolyl.

Some examples of substituents of the above-described aliphatic (and other) moieties of compounds of the invention include, but are not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

In general, the terms “aryl” and “heteroaryl”, as used herein, refer to stable mono- or polycyclic, heterocyclic, polycyclic, and polyheterocyclic unsaturated moieties having preferably 3-14 carbon atoms, each of which may be substituted or unsubstituted. Substituents include, but are not limited to, any of the previously mentioned substitutents, i.e., the substituents recited for aliphatic moieties, or for other moieties as disclosed herein, resulting in the formation of a stable compound. In certain embodiments of the present invention, “aryl” refers to a mono- or bicyclic carbocyclic ring system having one or two aromatic rings including, but not limited to, phenyl, naphthyl, tetrahydronaphthyl, indanyl, indenyl, and the like. In certain embodiments of the present invention, the term “heteroaryl”, as used herein, refers to a cyclic aromatic radical having from five to ten ring atoms of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl,oxadiazolyl, thiophenyl, furanyl, quinolinyl, isoquinolinyl, and the like.

It will be appreciated that aryl and heteroaryl groups can be unsubstituted or substituted, wherein substitution includes replacement of one, two, three, or more of the hydrogen atoms thereon independently with any one or more of the following moieties including, but not limited to: aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of other aliphatic, heteroaliphatic, or heterocyclic moieties, may optionally be substituted with substituents including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The term “heteroaliphatic”, as used herein, refers to aliphatic moieties that contain one or more oxygen, sulfur, nitrogen, phosphorus, or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The terms “halo” and “halogen” as used herein refer to an atom selected from fluorine, chlorine, bromine, and iodine.

The term “haloalkyl” denotes an alkyl group, as defined above, having one, two, or three halogen atoms attached thereto and is exemplified by such groups as chloromethyl, bromoethyl, trifluoromethyl, and the like.

The term “heterocycloalkyl” or “heterocycle”, as used herein, refers to a non-aromatic 5-, 6-, or 7-membered ring or a polycyclic group, including, but not limited to a bi- or tri-cyclic group comprising fused six-membered rings having between one and three heteroatoms independently selected from oxygen, sulfur and nitrogen, wherein (i) each 5-membered ring has 0 to 1 double bonds and each 6-membered ring has 0 to 2 double bonds, (ii) the nitrogen and sulfur heteroatoms may be optionally be oxidized, (iii) the nitrogen heteroatom may optionally be quaternized, and (iv) any of the above heterocyclic rings may be fused to a benzene ring. Representative heterocycles include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. In certain embodiments, a “substituted heterocycloalkyl or heterocycle” group is utilized and as used herein, refers to a heterocycloalkyl or heterocycle group, as defined above, substituted by the independent replacement of one, two or three of the hydrogen atoms thereon with but are not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples which are described herein.

“Carbocycle”: The term “carbocycle”, as used herein, refers to an aromatic or non-aromatic ring in which each atom of the ring is a carbon atom.

“Independently selected”: The term “independently selected” is used herein to indicate that the R groups can be identical or different.

“Labeled”: As used herein, the term “labeled” is intended to mean that a compound has at least one element, isotope, or chemical compound attached to enable the detection of the compound. In general, labels typically fall into three classes: a) isotopic labels, which may be radioactive or heavy isotopes, including, but not limited to, ²H, ³H, ³²P, ³⁵S, ⁶⁷Ga, ^{99m}Tc (Tc-99m), ¹¹¹In, ¹²³I, ¹²⁵I, ¹⁶⁹Yb and ¹⁸⁶Re; b) immune labels, which may be antibodies or antigens, which may be bound to enzymes (such as horseradish peroxidase) that produce detectable agents; and c) colored, luminescent, phosphorescent, or fluorescent dyes. It will be appreciated that the labels may be incorporated into the compound at any position that does not interfere with the biological activity or characteristic of the compound that is being detected. In certain embodiments of the invention, photoaffinity labeling is utilized for the direct elucidation of intermolecular interactions in biological systems. A variety of known photophores can be employed, most relying on photoconversion of diazo compounds, azides, or diazirines to nitrenes or carbenes (See, Bayley, H., Photogenerated Reagents in Biochemistry and Molecular Biology (1983), Elsevier, Amsterdam.), the entire contents of which are hereby incorporated by reference. In certain embodiments of the invention, the photoaffinity labels employed are o-, m- and p-azidobenzoyls, substituted with one or more halogen moieties, including, but not limited to 4-azido-2,3,5,6-tetrafluorobenzoic acid.

The term “heterocyclic”, as used herein, refers to a non-aromatic partially unsaturated or fully saturated 3- to 10-membered ring system, which includes single rings of 3 to 8 atoms in size and bi- and tri-cyclic ring systems which may include aromatic six-membered aryl or aromatic heterocyclic groups fused to a non-aromatic ring. These heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized.

The term “heteroaryl”, as used herein, refers to a cyclic aromatic radical having from five to ten ring atoms of which one ring atom is selected from sulfur, oxygen, and nitrogen; zero, one, or two ring atoms are additional heteroatoms independently selected from sulfur, oxygen, and nitrogen; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

Specific heterocyclic and aromatic heterocyclic groups that may be included in the compounds of the invention include: 3-methyl-4-(3-methylphenyl)piperazine, 3 methylpiperidine, 4-(bis-(4-fluorophenyl)methyl)piperazine, 4-(diphenylmethyl)piperazine, 4-(ethoxycarbonyl)piperazine, 4-(ethoxycarbonylmethyl)piperazine, 4-(phenylmethyl)piperazine, 4-(1-phenylethyl)piperazine, 4-(1,1-dimethylethoxycarbonyl)piperazine, 4-(2-(bis-(2-propenyl)amino)ethyl)piperazine, 4-(2-(diethylamino)ethyl)piperazine, 4-(2-chlorophenyl)piperazine, 4-(2-cyanophenyl)piperazine, 4-(2-ethoxyphenyl)piperazine, 4-(2-ethylphenyl)piperazine, 4-(2-fluorophenyl)piperazine, 4-(2-hydroxyethyl)piperazine, 4-(2-methoxyethyl)piperazine, 4-(2-methoxyphenyl)piperazine, 4-(2-methylphenyl)piperazine, 4-(2-methylthiophenyl)piperazine, 4-(2-nitrophenyl)piperazine, 4-(2-nitrophenyl)piperazine, 4-(2-phenylethyl)piperazine, 4-(2-pyridyl)piperazine, 4-(2-pyrimidinyl)piperazine, 4-(2,3-dimethylphenyl)piperazine, 4-(2,4-difluorophenyl)piperazine, 4-(2,4-dimethoxyphenyl)piperazine, 4-(2,4-dimethylphenyl)piperazine, 4-(2,5-dimethylphenyl)piperazine, 4-(2,6-dimethylphenyl)piperazine, 4-(3-chlorophenyl)piperazine, 4-(3-methylphenyl)piperazine, 4-(3-trifluoromethylphenyl)piperazine, 4-(3,4-dichlorophenyl)piperazine, 4-3,4-dimethoxyphenyl)piperazine, 4-(3,4-dimethylphenyl)piperazine, 4-(3,4-methylenedioxyphenyl)piperazine, 4-(3,4,5-trimethoxyphenyl)piperazine, 4-(3,5-dichlorophenyl)piperazine, 4-(3,5-dimethoxyphenyl)piperazine, 4-(4-(phenylmethoxy)phenyl)piperazine, 4-(4-(3,1-dimethylethyl)phenylmethyl)piperazine, 4-(4-chloro-3-trifluoromethylphenyl)piperazine, 4-(4-chlorophenyl)-3-methylpiperazine, 4-(4-chlorophenyl)piperazine, 4-(4-chlorophenyl)piperazine, 4-(4-chlorophenylmethyl)piperazine, 4-(4-fluorophenyl)piperazine, 4-(4-methoxyphenyl)piperazine, 4-(4-methylphenyl)piperazine, 4-(4-nitrophenyl)piperazine, 4-(4-trifluoromethylphenyl)piperazine, 4-cyclohexylpiperazine, 4-ethylpiperazine, 4-hydroxy-4-(4-chlorophenyl)methylpiperidine, 4-hydroxy-4-phenylpiperidine, 4-hydroxypyrrolidine, 4-methylpiperazine, 4-phenylpiperazine, 4-piperidinylpiperazine, 4-(2-furanyl)carbonyl)piperazine, 4-((1,3-dioxolan-5-yl)methyl)piperazine, 6-fluoro-1,2,3,4-tetrahydro-2-methylquinoline, 1,4-diazacylcloheptane, 2,3-dihydroindolyl, 3,3-dimethylpiperidine, 4,4-ethylenedioxypiperidine, 1,2,3,4-tetrahydroisoquinoline, 1,2,3,4-tetrahydroquinoline, azacyclooctane, decahydroquinoline, piperazine, piperidine, pyrrolidine, thiomorpholine, and triazole.

The terms “substituted,” whether preceded by the term “optionally” or not, and “substituent”, as used herein, refer to the ability, as appreciated by one skilled in this art, to change one functional group for another functional group provided that the valency of all atoms is maintained. When more than one position in any given structure may be substituted with more than one substituent selected from a specified group, the substituent may be either the same or different at every position. The substituents may also be further substituted (e.g., an aryl group substituent may have another substituent off it, such as another aryl group, which is further substituted with fluorine at one or more positions).

The following are more general terms used throughout the present application:

“Animal”: The term animal, as used herein, refers to humans as well as non-human animals, including, for example, mammals, birds, reptiles, amphibians, and fish. Preferably, the non-human animal is a mammal (e.g., a rodent, a mouse, a rat, a rabbit, a monkey, a dog, a cat, a primate, or a pig). An animal may be a transgenic animal.

“Associated with”: When two entities are “associated with” one another as described herein, they are linked by a direct or indirect covalent or non-covalent interaction. Preferably, the association is covalent. Desirable non-covalent interactions include hydrogen bonding, van der Waals interactions, hydrophobic interactions, magnetic interactions, electrostatic interactions, etc.

“Biocompatible”: The term “biocompatible”, as used herein is intended to describe compounds that are not toxic to cells. Compounds are “biocompatible” if their addition to cells in vitro results in less than or equal to 20% cell death, and their administration in vivo does not induce inflammation or other such adverse effects.

“Biodegradable”: As used herein, “biodegradable” compounds are those that, when introduced into cells, are broken down by the cellular machinery or by hydrolysis into components that the cells can either reuse or dispose of without significant toxic effect on the cells (i.e., fewer than about 20% of the cells are killed when the components are added to cells in vitro). The components preferably do not induce inflammation or other adverse effects in vivo. In certain preferred embodiments, the chemical reactions relied upon to break down the biodegradable compounds are uncatalyzed.

“Effective amount”: In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the composition of the encapsulating matrix, the target tissue, etc. For example, the effective amount of microparticles containing an antigen to be delivered to immunize an individual is the amount that results in an immune response sufficient to prevent infection with an organism having the administered antigen.

“Immunostimulatory polynucleotide”: As used herein, an “immunostimulatory polynucleotide” is any polynucleotide that induces an immune response in a subject. The “immunostimulatory polynucleotide” directly induced an immune response in a subject and does not depend on a product (RNA or protein) or transcription or translation of the immunostimulatory polynucleotide. The immunostimulatory polynucleotide may be RNA, DNA, or a derivative thereof. In certain embodiments, the immunostimulatory polynucleotide binds to a Toll-like receptor. In certain embodiments, the immunostimulatory polynucleotide binds to a receptor on the surface of an immune cell (e.g., dendritic cell, macrophage, T-cell, B-cell, monocyte). Exemplary immunostimulatory polynucleotides includes is RNA, ssRNA, dsRNA, CpG sequences, unmethylated bacterial DNA, unmethylated viral DNA, CpG oligonucleotides, and CpG oligodeoxynucleotides.

“Peptide” or “protein”: According to the present invention, a “peptide” or “protein” comprises a string of at least three amino acids linked together by peptide bonds. The terms “protein” and “peptide” may be used interchangeably. Peptide may refer to an individual peptide or a collection of peptides. Inventive peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

“Polynucleotide” or “oligonucleotide” or “nucleic acid”: Polynucleotide, oligonucleotide, or nucleic acid refers to a polymer of nucleotides. Typically, a polynucleotide comprises at least three nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

“Small molecule”: As used herein, the term “small molecule” refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds. Known naturally-occurring small molecules include, but are not limited to, penicillin, erythromycin, taxol, cyclosporin, and rapamycin. Known synthetic small molecules include, but are not limited to, ampicillin, methicillin, sulfamethoxazole, and sulfonamides.

“siRNA”: As used herein, the term “siRNA” refers to small interfering RNA, also sometimes referred to as short interfering RNA or silencing RNA. siRNA is typical about 20-25 nucleotides long and double-stranded. siRNA is involved in RNA interference (RNAi), where it interferes with the expression of a certain gene. siRNA may also participate in other pathways, such as antiviral mechanisms and shaping the chromatin of a genome.

“is RNA”: As used herein, the term “is RNA” refers to immunostimulatory RNA. Immunostimulatory RNA is any RNA sequence that is capable of modulating an immune response. These RNA can be single stranded, short RNA that interact with TLR7 and TLR8 or other unknown receptors, or other longer single stranded RNA that interact with cytosolic receptors such as RIG-I, MDA, PKR, or other unknown receptors. is RNA can also be double-stranded RNA such as siRNA, or longer dsRNA, that interact with TLR7, TLR8, and/or TLR3 or other receptors. The term is RNA is generally a functional definition rather than structural, though structure and sequence of the RNA plays a role.

“CpG DNA”: As used herein, the term “CpG DNA” refers to a DNA fragment with a sequence containing cytosine and guanine. CpG DNAs mimic non-methylated bacterial and viral DNA and are recognized by toll-like receptors (TLRs) to stimulate an immune response (Krieg et al., Nature (2002) 374: 546-549; which is incorporated herein by reference).

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

### BRIEF DESCRIPTION OF THE DRAWINGS

FIG. 1. Acrylates, acrylamides, and amines used in the synthesis of exemplary lipidoids. Parent compounds of lipidoid library for is RNA delivery. A subset of components from the larger lipidoid library²⁶ used in this study. When combined with amine-containing cores to form lipid-like structures, alkyl-acrylate (L) tails form a hydrolysable ester bond, and alkyl-acrylamides (N) form non-degradable amine linkages. Tail groups are coded according to linkage and number of carbons in the alkyl chain.

FIG. 2. Schematic of solvent-free batch synthesis process resulting in crude mixtures of lipidoid components. All reactions were performed in excess of tail groups to drive towards full substitution of all core amine groups. Primary amines may accept up to two tail substitutions and secondary amines can accept up to one. Crude products typically contain a mixture of fully n-substituted and n−1 substituted lipidoids with rare n−2 substitutions.

FIG. 3. List of the 96 lipidoid compositions screened for is RNA delivery.

FIG. 4. Results of in vitro is RNA delivery screen of lipidoid compositions.

FIG. 5. Screening highlights is RNA delivery in vitro with lipidoids prepared with amine 100, also referred to herein as 100-core lipidoids. Type 1 interferon activity following human PBMC transfection with 100-core shown for all four weight ratios of lipidoid to RNA using immunostimulatory RNA R1362. Type 1 interferon activity was normalized to Lipofectamine 2000 delivery of R1362. Activity in vitro of all 100-core materials complexed with control RNA R1263 was near or below detection limits. Sequence of R1362: 5′-UUGUUGUUGUUGUUGUUGUU-3′. Sequence of R1263: 5′-GCCACCGAGCCGAAGGCACC-3′

FIG. 6. Structures and synthetic scheme of 100-core lipidoid materials. Second generation lipidoid materials were designed based on the 100 core and 10 to 12 carbon alkyl-acrylamide tails with structures as shown. These lipidoids were synthesized and purified to single isomer components.

FIG. 7. is RNA delivery properties of 100-core lipidoid materials. Purified lipidoids and second generation 100-core materials were screened in vitro for R1362 is RNA delivery with R1263RNA as a control. Type 1 interferon activity is normalized to Lipofectamine 2000 with R1362 (dotted line).

FIG. 8. Dose-dependent inhibition of influenza viral replication in mouse lung.

FIG. 9. Inhibitory effect of lipidoid nanoparticles correlates with induction of systemic Type 1 interferon response.

FIG. 10. All 96 lipidoid compounds were screened for is RNA delivery in vitro at four different mass ratios of lipidoid to RNA (15, 10, 5, 2.5 to 1), with an immunostimulatory RNA (R1362) and control RNA (R1263) comprising over 900 unique transfection experiments. Complexes were added to 5×10⁵ human PBMCs in 96 well plates at 200 ng RNA per well (1 μg/mL˜140 nM) for 16-20 hours. Type I interferon activity was determined by cell-based 293T-ISRE-RFP assay and activity was normalized for each batch of PBMCs to activity of Lipofectamine 2000 (L2K) complexed with R1362 (grey bar, dotted line). The highest relative Type I IFN activity per unique compound is shown for all 96 lipidoids, with 100-core lipidoids indicated by black bars. Error bars represent standard deviation, n=4.

FIG. 11. Structures and in vitro is RNA delivery characterization of second generation lipidoids based on 100 core. (A) Second generation lipidoids were designed based on the 100 core, synthesized with 10 to 12 carbon alkyl-acrylamide tails, and purified into single isomer components. (B) Purified lipidoids were screened in vitro for R1362 is RNA delivery with R1263 RNA as a control. Type I interferon activity is normalized to transfection of R1362 with Lipofectamine 2000 (dotted line).

FIG. 12. In vivo screening for activation of innate immune responses following injection of formulated lipidoid-RNA nanoparticles. Lipidoid-RNA nanoparticles formulated with 100 ug R1362 RNA were injected SC in BALB/c mice (n=3 or 4). R1362 formulated with DOTAP and a mock injection with HBSS were included as controls. (A) First round screening with lyophilized nanoparticles resuspended in HBSS. (B) Second round screening with nanoparticles dialyzed against HBSS for 2 hours. (C) Comparison of lyophilized and dialyzed nanoparticles at either 10:1 or 15:1 w:w ratio of lipidoid:RNA. (A-C) Blood was collected at 6, 9, 12, and 24 hours following injection. Serum interferon-alpha, IP-10, and IL-6 cytokine levels were measured. (D) Comparison of lyophilized and dialyzed nanoparticles at either 10:1 or 15:1 w:w ratio of lipidoid:RNA. Spleens were also collected at 24 hours for staining and FACS analysis of CD3⁺ T-cell activation, CD19⁺ B-cell activation and maturation, and DX5⁺ NK-cell activation. Cytolytic activity of NK-cells was measured by chromium release following incubation of spleenocytes with YAC-1 target cells.

FIG. 13. Characterization of cytokine response from B and D lipidoid-RNA nanoparticles in vivo. Lipidoids B [ND(2)NA(2)-100] and D [ND(2)LD(2)-100] were formulated with the strong TLR7/8 agonist R1362 RNA and the weak TLR7/8 agonist R1263 RNA as control in dialyzed form. (A) Dose-response of IFN-alpha (top), IP-10 (middle), and IL-6 (bottom) at 9 and 12 hours following SC injection into 129sv mice (n=4) at 3, 10, 30, and 100 μg of nanoparticle-encapsulated R1362 RNA in lipidoid B (left) or D (right, note scale change for IFN-alpha and IP-10). (B) Serum cytokine response of IFN-alpha (top), IP-10 (middle) and IL-6 (bottom) at 6 hours following IV injection of 50 μg RNA into MyD88^{-/-} or TLR7^{-/-} mice, or 30 μg RNA into TLR9^{-/-} or TLR4^{-/-} mice (n=3 or 4).

FIG. 14. Prophylaxis against influenza infection.

FIG. 15. Adjuvant Activity in Protein Vaccination. *=significant to p=0.05 by 1-way ANOVA with Dunn's post-test. **=significant to p=0.05 by 1-way ANOVA with Dunn's post-test.

FIG. 16. Lipidoids can be further reacted with methyl iodide to form quaternary amines carrying a permanent positive charge. These materials are coded with the “Q” designation.

FIG. 17. (A) Activity of any formulation (lipidid, L:R ratio, or RNA) with relative activity above 0.5. Lipidoids with the 100 core are colored in red. Dashed line indicates relative activity of 1 compared to transfection with Lipofectamine 2000 and R1362. Each bar is the average of 4 repeats with standard deviation. (B) Screening highlights immunostimulatory RNA delivery in vitro with 100-core lipidoids. Type I interferon activity following human PBMC transfection with 100 core shown for all four weight ratios of lipidoid to RNA using immunostimulatory RNA R1362. Type I interferon activity was normalized to Lipofectamine 2000 delivery of R1362. Activity in vitro of all 100-core materials complexed with control RNA R1263 was near or below detection limits (data not shown). Lipidoid NH100 was insoluble in sodium acetate even after heating and sonication. While NF100 had the highest activity of all lipidoids, the degradable version, LF100, was not as active. Further, quaternization (FIG. 16) of the degradable version of NG100 severely reduced delivery potential.

FIG. 18. Synthesis of second generation lipidoids based on the 100-core. Intermediate protection and deprotection steps were used to generate the ND(2)-100 precursor, which was further reacted with the NA or LD tails in the synthesis of mixed-tail lipidoids of the 100 core.

FIG. 19. Comparison of cytokine response between R1362 and R1263 RNA. Lipidoids B [ND(2)NA(2)-100] and D [ND(2)LD(2)-100] were formulated with the strong TLR7/8 agonist R1362 RNA as well as the weak TLR7/8 agonist R1263 RNA as control in liquid form. Serum cytokine responses of IFN-alpha (left), IP-10 (middle), and IL-6 (right) following IV injection into 129sc mice (n=4) of 75 μg of active R1362 RNA or control R1263 RNA formulated in lipidoid nanoparticles at 3, 6, and 9 hours after injection.

FIG. 20. In vitro stimulation of HEK293T cell lines stably expressing TLRs.

FIG. 21. Antibody titers by IgG classes (IgG1 and IgG2c).

FIG. 22. Characterization of cytokine response of spleenocytes following vaccination and in vitro restimulation.

FIG. 23. (A) Immunostimulatory effect of lipidoids K1-K34 using R1362 is RNA as determined by interferon-alpha concentration. Lipofectamine 2000 (L2K) delivery of R1362 is RNA is also shown. Note tha K32 is equivalent to ND/NA-100 as described in the Examples. (B) Lipidoids K1-K34 with their respective components. (C) Scheme of the synthesis of core 98 and 100 lipidoids with the possibility of mixed tails.

### DETAILED DESCRIPTION OF THE INVENTION

The present invention provides lipidoids that can be used with an immunostimulatory polynucleotide (e.g., is RNA) to modulate the immune system of a subject. The structure and function of lipidoids as delivery materials for RNA interference (RNAi) have been broadly described (Akinc et al., Nat. Biotechnol. (2008) 26: 561-569; U.S. patent application U.S. Ser. No. 11/453,222, filed Jun. 14, 2006, and international PCT patent application PCT/US06/23171, publication number WO/2006/138380, filed Jun. 14, 2006, which claim priority to U.S. provisional application 60/785,176, filed Mar. 23, 2006, and U.S. provisional application 60/690,608, filed Jun. 15, 2005, all of which are incorporated herein by reference). Various lipidoids have been synthesized, and specific structures that are useful for modulating an immune response have been identified. New formulations of lipidoid-based compositions and particles have been prepared, and new uses for lipidoids in immune modulation have been developed.

The lipidoids of the present invention are useful in drug delivery, specifically the delivery of polynucleotides (e.g., is RNA). The lipidoids with their amine-containing hydrophilic portion may be used to complex polynucleotides and thereby enhance the delivery of the polynucleotide and/or prevent its degradation. The lipidiod itself may in certain instances have a non-specific adjuvant activity. In certain embodiments, the lipidoid has an RNA-specific adjuvant activity. The lipidoids may also be used in the formation of nanoparticles containing a polynucleotide. Preferably, the lipidoids are biocompatible and biodegradable, and the formed particles are also biodegradable and biocompatible and may be used to provide controlled, sustained release of the polynucleotide. The lipidoids and their corresponding particles may also be responsive to pH changes given that these compounds are protonated at lower pH.

Currently, there are no available methods for the efficient in vivo delivery of immunostimulatory polynucleotides (e.g., is RNA). Delivery of immunostimulatory nucleic acids has mostly been confined to large double-stranded RNA (such as poly(I:C)RNA) and DNA (such as plasmid DNA for DNA vaccines, and CpG DNA oligonucleotides for vaccine adjuvants). Small RNAs, such as siRNAs, can be immunostimulatory depending on sequence, chemical structure, and intracellular location (Eberle et al., J Immunol (2008) 180: 3229-3237; Sioud, et al., Trends Mol Med (2006) 12: 167-176; each of which is incorporated herein by reference). These types of polynucleotides can activate Toll-like receptors, such as TLR3, TLR7, TLR8, and TLR9. The therapeutic use of these immunostimulatory polynucleotides depends on delivery of the polynucleotide to the correct cell type and intracellular location. In certain embodiments, the lipidoid aids in the delivery of the immunstimulatory polynucleotide to the endosome of a cell of the immune system (e.g., dendritic cell, monocyte).

The lipidoids of the current invention are able to functionally deliver immunomodulatory polynucleotides (e.g., single-stranded and double-stranded RNA; CpG DNA oligonucleotides). The present invention provides new uses of lipidoids not previously described.

### Lipidoids

The lipidoids of the present invention are lipids containing primary, secondary, tertiary, or quaternary amines, and salts thereof. In some embodiments, the inventive lipidoids are relatively non-cytotoxic. In some embodiments, the inventive lipidoids are biocompatible and biodegradable. In certain embodiments, the inventive lipidoids are immunogenic, that is, aid in the stimulation of a desired immune response. In some embodiments, the lipidoids of the present invention have pKₐs in the range of 5.5 to 7.5, more preferably between 6.0 and 7.0. In some embodiments, the lipidoid may be designed to have a desired pKₐ between 3.0 and 9.0, more preferably between 5.0 and 8.0. The inventive lipidoids are particularly attractive for delivery of polynucleotides for several reasons: 1) they contain amino groups for interacting with DNA, RNA, and other polynucleotides, for buffering the pH, for causing endosomolysis, etc.; 2) they can be synthesized from commercially available starting materials; and 3) they are pH responsive and can be engineered with a desired pKₐ. The lipidoids have also been found to have both RNA-specific and non-specific adjuvant activity.

Any lipidoid may be used to deliver immunostimulatory polynucleotides. Lipidoids have been previously described in Akinc et al., Nat. Biotechnol. (2008) 26:561-569; U.S. patent application U.S. Ser. No. 11/453,222, filed Jun. 14, 2006, and international PCT patent application PCT/US06/23171, publication number WO/2006/138380, filed Jun. 14, 2006, which claim priority to U.S. provisional application 60/785,176, filed Mar. 23, 2006, and U.S. provisional application 60/690,608, filed Jun. 15, 2005. Any such lipidoids may be used to deliver polynucleotides, particularly immunostimulatory polynucletides. In certain embodiments, lipidoids that are useful in delivering immunostimulatory polynucleotides are of the formula (I):

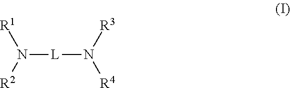

wherein

L is a substituted or unsubstituted alkylene group having between one and six carbon atoms, inclusive;

R¹, R², R³, and R⁴ are independently selected from the group consisting of hydrogen, C₁-C₆ alkyl, C₃-C₇ cycloalkyl, —(CH₂)ₙOR^{A}, —(CH₂)ₙN(R^{A})₂, Y, —(CH₂)ₙN(Y)₂, —(CH₂)ₙNR^{A}Y; wherein each occurrence of R^{A} is independently a hydrogen; a protecting group; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted aryl; substituted or unsubstituted heteroaryl; alkoxy; aryloxy; alkylthio; arylthio; amino; alkylamino; dialkylamino; heteroaryloxy; or heteroarylthio moiety; with the proviso that at least one of R¹, R², R³, and R⁴ must equal Y, —(CH₂)ₙN(Y)₂, or —(CH₂)ₙNR^{A}Y;

R¹ and R² may be taken together with the intervening atoms to form a cyclic structure;

R³ and R⁴ may be taken together with the intervening atoms to form a cyclic structure;

each occurrence of Y is independently selected from the group consisting of —CH₂CH₂CO₂R⁶ or —CH₂CH₂CONHR⁶; wherein each occurrence of R⁶ is independently an alkyl chain of 6-20 carbon atoms, inclusive;

each occurrence of n is independently an integer between one and six, inclusive; and salts thereof.

In certain embodiments, the tertiary amine of formula (I) is protonated or alkylated to form a compound of formula (Ia):

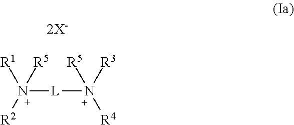

wherein L, R¹, R², R³, and R⁴ are defined as described herein;

R⁵ is hydrogen or C₁-C₆ aliphatic; and

X⁻ is an anion. Possible anions include fluoride, chloride, bromide, iodide, sulfate, bisulfate, phosphate, nitrate, acetate, fumarate, oleate, citrate, valerate, maleate, oxalate, isonicotinate, lactate, salicylate, tartrate, tannate, pantothenate, bitartrate, ascorbate, succinate, gentisinate, gluconate, glucaronate, saccharate, formate, benzoate, glutamate, methanesulfonate, ethanesulfonate, benzenesulfonate, p-toluenesulfonate, pamoate (i.e., 1,1′-methylene-bis-(2-hydroxy-3-naphthoate), or a polynucleotide.

In certain embodiments, lipidoids that are useful in delivering immunostimulatory polynucleotides are of the formula (II):

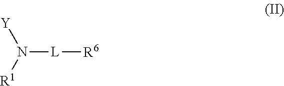

wherein L, R¹, Y, and n are defined as described herein;

R⁶ is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N₃; —N(R_{A})₂; —NHC(═O)R_{A}; —NR_{A}C(═O)N(R_{A})₂; —OC(═O)OR_{A}; —OC(═O)R_{A}; —OC(═O)N(R_{A})₂; —NR_{A}C(═O)OR_{A}; and —C(R_{A})₃; wherein each occurrence of R_{A} is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; and salts thereof.

In certain embodiments, the tertiary amine of formula (II) is protonated or alkylated to form a compound of formula (IIa):

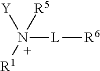

wherein L, R¹, Y, R⁵, R⁶, and X⁻ are defined as described herein.

In certain embodiments, lipidoids that are useful in delivering immunostimulatory polynucleotides are of the formula (III):

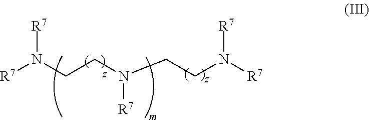

wherein

each occurrence of z is an integer between 1 and 10, inclusive;

m is an integer between 0 and 10, inclusive;

each occurrence of R₇ is independently hydrogen or Y;

each occurrence of Y is independently selected from the group consisting of —CH₂CH₂CO₂R⁴ or —CH₂CH₂CONHR⁴; wherein each occurrence of R⁴ is independently an alkyl chain of 6-20 carbon atoms, inclusive; and salts thereof.

In certain embodiments, the tertiary amine of formula (III) is protonated or alkylated to form a compound of formula (IIIa):

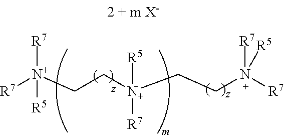

wherein z, m, R⁵, R⁷, and X⁻ are defined as described herein.

In some embodiments, L is an alkylene group having 2-6 carbon atoms. In certain embodiments, L is an alkylene group having 2-4 carbon atoms. In certain embodiments, L is an ethylene group. In certain embodiments, L is a propylene group. In certain embodiments, L is a butylene group.

In certain embodiments, at least one of R¹, R², R³, and R⁴ is hydrogen. In certain embodiments, only one of R¹, R², R³, and R⁴ is hydrogen. In certain embodiments, at least two of R¹, R², R³, and R⁴ are hydrogen. In certain embodiments, three of R¹, R², R³, and R⁴ are hydrogen.

In certain embodiments, R¹, R², R³, and R⁴ are not the same. In certain embodiments, R¹, R², R³, and R⁴ are all the same. In certain embodiments, at least two of R¹, R², R³, and R⁴ are the same. In certain embodiments, at least three of R¹, R², R³, and R⁴ are the same. In certain embodiments, at least one of R¹, R², R³, and R⁴ are not the same. In certain embodiments, at least two of R¹, R², R³, and R⁴ are not the same. In certain embodiments, at least three of R¹, R², R³, and R⁴ are not the same. In certain embodiments, all of R¹, R², R³, and R⁴ are not the same.

In certain embodiments, at least one instance of R⁷ is hydrogen. In certain embodiments, only one instance of R⁷ is hydrogen. In certain embodiments, at least two instances of R⁷ are hydrogen. In certain embodiments, at least three instances of R⁷ are hydrogen. In certain embodiments, at least four instances of R⁷ are hydrogen. In certain embodiments, at least five instances of R⁷ are hydrogen.

In certain embodiments, all instances of R⁷ are not the same. In certain embodiments, each instance of R⁷ is the same. In certain embodiments, at least two instances of R⁷ are the same. In certain embodiments, at least three instances of R⁷ are the same. In certain embodiments, at least four instances of R⁷ are the same. In certain embodiments, at least five instances of R⁷ are the same.

In certain embodiments, R¹ is hydrogen. In other embodiments, R¹ is C₁-C₆ alkyl or C₃-C₇ cycloalkyl. In certain embodiments, R¹ is taken together with R² and the intervening atoms to form a ring. In certain embodiments, the ring formed by R¹, R², and the intervening atoms is pyrrolidine, piperidine, morpholine, or homopiperidine. In certain embodiments, R¹ is —(CH₂)ₙOR^{A} or —(CH₂)ₙN(R^{A})₂. In certain embodiments, R¹ is —(CH₂)ₙOR^{A}. In certain embodiments, R¹ is —(CH₂)₂OH. In certain embodiments, R¹ is Y. In certain embodiments, R¹ is —(CH₂)ₙN(Y)₂ or —(CH₂)ₙNR^{A}Y. In other embodiments, R¹ is —CH₂CH₂CO₂R⁸ or —CH₂CH₂CONHR⁸. In certain embodiments, R¹ is —CH₂CH₂CO₂R⁸. In certain embodiments, R¹ is —CH₂CH₂CONHR⁸. In certain embodiments, R⁸ is an unsubstituted, straight chain alkyl group with at least 6 carbons. In certain embodiments, R⁸ is an unsubstituted, straight chain alkyl group, C₆-C₃₀. In certain embodiments, R⁸ is an unsubstituted, straight chain alkyl group, preferably C₉-C₂₀. In certain embodiments, R¹ is —CH₂CH₂CO₂R⁸, wherein R⁸ is an unsubstituted, unbranched C₉ alkyl chain. In certain embodiments, R¹ is —CH₂CH₂CO₂R⁸, wherein R⁸ is an unsubstituted, unbranched C₁₀ alkyl chain. In certain embodiments, R¹ is —CH₂CH₂CO₂R⁸, wherein R⁸ is an unsubstituted, unbranched C₁₁, alkyl chain. In certain embodiments, R¹ is —CH₂CH₂CO₂R⁸, wherein R⁸ is an unsubstituted, unbranched C₁₂ alkyl chain. In certain embodiments, R¹ is —CH₂CH₂CO₂R⁸, wherein R⁸ is an unsubstituted, unbranched C₁₃ alkyl chain. In certain embodiments, R¹ is —CH₂CH₂CO₂R⁸, wherein R⁸ is an unsubstituted, unbranched C₁₄ alkyl chain. In certain embodiments, R¹ is —CH₂CH₂CO₂R⁸, wherein R⁸ is an unsubstituted, unbranched C₁₅ alkyl chain. In certain embodiments, R¹ is —CH₂CH₂CO₂R⁸, wherein R⁸ is an unsubstituted, unbranched C₁₆ alkyl chain. In certain embodiments, R¹ is —CH₂CH₂CO₂R⁸, wherein R⁸ is an unsubstituted, unbranched C₁₇ alkyl chain. In certain embodiments, R¹ is —CH₂CH₂CO₂R⁸, wherein R⁸ is an unsubstituted, unbranched C₁₈ alkyl chain. In certain embodiments, R¹ is —CH₂CH₂CO₂R⁸, wherein R⁸ is an unsubstituted, unbranched C₁₉ alkyl chain. In certain embodiments, R¹ is —CH₂CH₂CO₂R⁸, wherein R⁸ is an unsubstituted, unbranched C₂₀ alkyl chain. In certain embodiments, R¹ is —CH₂CH₂CONHR⁸, wherein R⁸ is an unsubstituted, unbranched C₉ alkyl chain. In certain embodiments, R¹ is —CH₂CH₂CONHR⁸, wherein R⁸ is an unsubstituted, unbranched C₁₀ alkyl chain. In certain embodiments, R¹ is —CH₂CH₂CONHR⁸, wherein R⁸ is an unsubstituted, unbranched C₁₁ alkyl chain. In certain embodiments, R¹ is —CH₂CH₂CONHR⁸, wherein R⁸ is an unsubstituted, unbranched C₁₂ alkyl chain. In certain embodiments, R¹ is —CH₂CH₂CONHR⁸, wherein R⁸ is an unsubstituted, unbranched C₁₃ alkyl chain. In certain embodiments, R¹ is —CH₂CH₂CONHR⁸, wherein R⁸ is an unsubstituted, unbranched C₁₄ alkyl chain. In certain embodiments, R¹ is —CH₂CH₂CONHR⁸, wherein R⁸ is an unsubstituted, unbranched C₁₅ alkyl chain. In certain embodiments, R¹ is —CH₂CH₂CONHR⁸, wherein R⁸ is an unsubstituted, unbranched C₁₆ alkyl chain. In certain embodiments, R¹ is —CH₂CH₂CONHR⁸, wherein R⁸ is an unsubstituted, unbranched C₁₇ alkyl chain. In certain embodiments, R¹ is —CH₂CH₂CONHR⁸, wherein R⁸ is an unsubstituted, unbranched C₁₈ alkyl chain. In certain embodiments, R¹ is —CH₂CH₂CONHR⁸, wherein R⁸ is an unsubstituted, unbranched C₁₉ alkyl chain. In certain embodiments, R¹ is —CH₂CH₂CONHR⁸, wherein R⁸ is an unsubstituted, unbranched C₂₀ alkyl chain.

In certain embodiments, R² is hydrogen. In other embodiments, R² is C₁-C₆ alkyl or C₃-C₇ cycloalkyl. In certain embodiments, R² is taken together with R¹ and the intervening atoms to form a ring. In certain embodiments, the ring formed by R¹, R², and the intervening atoms is pyrrolidine, piperidine, morpholine, or homopiperidine. In certain embodiments, R² is —(CH₂)ₙOR^{A} or —(CH₂)ₙN(R^{A})₂. In certain embodiments, R² is —(CH₂)ₙOR^{A}. In certain embodiments, R² is —(CH₂)₂OH. In certain embodiments, R² is Y. In certain embodiments, R² is —(CH₂)ₙN(Y)₂ or —(CH₂)ₙNR^{A}Y. In other embodiments, R² is —CH₂CH₂CO₂R⁸ or —CH₂CH₂CONHR⁸. In certain embodiments, R² is —CH₂CH₂CO₂R⁸. In certain embodiments, R² is —CH₂CH₂CONHR⁸. In certain embodiments, R⁸ is an unsubstituted, straight chain alkyl group with at least 6 carbons. In certain embodiments, R⁸ is an unsubstituted, straight chain alkyl group, C₆-C₃₀. In certain embodiments, R⁸ is an unsubstituted, straight chain alkyl group, preferably C₉-C₂₀. In certain embodiments, R² is —CH₂CH₂CO₂R⁸, wherein R⁸ is an unsubstituted, unbranched C₉ alkyl chain. In certain embodiments, R² is —CH₂CH₂CO₂R⁸, wherein R⁸ is an unsubstituted, unbranched C₁₀ alkyl chain. In certain embodiments, R² is —CH₂CH₂CO₂R⁸, wherein R⁸ is an unsubstituted, unbranched C₁₁ alkyl chain. In certain embodiments, R² is —CH₂CH₂CO₂R⁸, wherein R⁸ is an unsubstituted, unbranched C₁₂ alkyl chain. In certain embodiments, R² is —CH₂CH₂CO₂R⁸, wherein R⁸ is an unsubstituted, unbranched C₁₃ alkyl chain. In certain embodiments, R² is —CH₂CH₂CO₂R⁸, wherein R⁸ is an unsubstituted, unbranched C₁₄ alkyl chain. In certain embodiments, R² is —CH₂CH₂CO₂R⁸, wherein R⁸ is an unsubstituted, unbranched C₁₅ alkyl chain. In certain embodiments, R² is —CH₂CH₂CO₂R⁸, wherein R⁸ is an unsubstituted, unbranched C₁₆ alkyl chain. In certain embodiments, R² is —CH₂CH₂CO₂R⁸, wherein R⁸ is an unsubstituted, unbranched C₁₇ alkyl chain. In certain embodiments, R² is —CH₂CH₂CO₂R⁸, wherein R⁸ is an unsubstituted, unbranched C₁₈ alkyl chain. In certain embodiments, R² is —CH₂CH₂CO₂R⁸, wherein R⁸ is an unsubstituted, unbranched C₁₉ alkyl chain. In certain embodiments, R² is —CH₂CH₂CO₂R⁸, wherein R⁸ is an unsubstituted, unbranched C₂₀ alkyl chain. In certain embodiments, R² is —CH₂CH₂CONHR⁸, wherein R⁸ is an unsubstituted, unbranched C₉ alkyl chain. In certain embodiments, R² is —CH₂CH₂CONHR⁸, wherein R⁸ is an unsubstituted, unbranched C₁₀ alkyl chain. In certain embodiments, R² is —CH₂CH₂CONHR⁸, wherein R⁸ is an unsubstituted, unbranched C₁₁ alkyl chain. In certain embodiments, R² is —CH₂CH₂CONHR⁸, wherein R⁸ is an unsubstituted, unbranched C₁₂ alkyl chain. In certain embodiments, R² is —CH₂CH₂CONHR⁸, wherein R⁸ is an unsubstituted, unbranched C₁₃ alkyl chain. In certain embodiments, R² is —CH₂CH₂CONHR⁸, wherein R⁸ is an unsubstituted, unbranched C₁₄ alkyl chain. In certain embodiments, R² is —CH₂CH₂CONHR⁸, wherein R⁸ is an unsubstituted, unbranched C₁₅ alkyl chain. In certain embodiments, R² is —CH₂CH₂CONHR⁸, wherein R⁸ is an unsubstituted, unbranched C₁₆ alkyl chain. In certain embodiments, R² is —CH₂CH₂CONHR⁸, wherein R⁸ is an unsubstituted, unbranched C₁₇ alkyl chain. In certain embodiments, R² is —CH₂CH₂CONHR⁸, wherein R⁸ is an unsubstituted, unbranched C₁₈ alkyl chain. In certain embodiments, R² is —CH₂CH₂CONHR⁸, wherein R⁸ is an unsubstituted, unbranched C₁₉ alkyl chain. In certain embodiments, R² is —CH₂CH₂CONHR⁸, wherein R⁸ is an unsubstituted, unbranched C₂₀ alkyl chain.

In certain embodiments, R³ is hydrogen. In other embodiments, R³ is C₁-C₆ alkyl or C₃-C₇ cycloalkyl. In certain embodiments, R³ is taken together with R⁴ and the intervening atoms to form a ring. In certain embodiments, the ring formed by R³, R⁴, and the intervening atoms is pyrrolidine, piperidine, morpholine, or homopiperidine. In certain embodiments, R³ is —(CH₂)ₙOR^{A} or —(CH₂)ₙN(R^{A})₂. In certain embodiments, R³ is —(CH₂)ₙOR^{A}. In certain embodiments, R³ is —(CH₂)₂OH. In certain embodiments, R³ is Y. In certain embodiments, R³ is —(CH₂)ₙN(Y)₂ or —(CH₂)ₙNR^{A}Y. In other embodiments, R³ is —CH₂CH₂CO₂R⁸ or —CH₂CH₂CONHR⁸. In certain embodiments, R³ is —CH₂CH₂CO₂R⁸. In certain embodiments, R³ is —CH₂CH₂CONHR⁸. In certain embodiments, R⁸ is an unsubstituted, straight chain alkyl group with at least 6 carbons. In certain embodiments, R⁸ is an unsubstituted, straight chain alkyl group, C₆-C₃₀. In certain embodiments, R⁸ is an unsubstituted, straight chain alkyl group, preferably C₉-C₂₀. In certain embodiments, R³ is —CH₂CH₂CO₂R⁸, wherein R⁸ is an unsubstituted, unbranched C₉ alkyl chain. In certain embodiments, R³ is —CH₂CH₂CO₂R⁸, wherein R⁸ is an unsubstituted, unbranched C₁₀ alkyl chain. In certain embodiments, R³ is —CH₂CH₂CO₂R⁸, wherein R⁸ is an unsubstituted, unbranched C₁₁ alkyl chain. In certain embodiments, R³ is —CH₂CH₂CO₂R⁸, wherein R⁸ is an unsubstituted, unbranched C₁₂ alkyl chain. In certain embodiments, R³ is —CH₂CH₂CO₂R⁸, wherein R⁸ is an unsubstituted, unbranched C₁₃ alkyl chain. In certain embodiments, R³ is —CH₂CH₂CO₂R⁸, wherein R⁸ is an unsubstituted, unbranched C₁₄ alkyl chain. In certain embodiments, R³ is —CH₂CH₂CO₂R⁸, wherein R⁸ is an unsubstituted, unbranched C₁₅ alkyl chain. In certain embodiments, R³ is —CH₂CH₂CO₂R⁸, wherein R⁸ is an unsubstituted, unbranched C₁₆ alkyl chain. In certain embodiments, R³ is —CH₂CH₂CO₂R⁸, wherein R⁸ is an unsubstituted, unbranched C₁₇ alkyl chain. In certain embodiments, R³ is —CH₂CH₂CO₂R⁸, wherein R⁸ is an unsubstituted, unbranched C₁₈ alkyl chain. In certain embodiments, R³ is —CH₂CH₂CO₂R⁸, wherein R⁸ is an unsubstituted, unbranched C₁₉ alkyl chain. In certain embodiments, R³ is —CH₂CH₂CO₂R⁸, wherein R⁸ is an unsubstituted, unbranched C₂₀ alkyl chain. In certain embodiments, R³ is —CH₂CH₂CONHR⁸, wherein R⁸ is an unsubstituted, unbranched C₉ alkyl chain. In certain embodiments, R³ is —CH₂CH₂CONHR⁸, wherein R⁸ is an unsubstituted, unbranched C₁₀ alkyl chain. In certain embodiments, R³ is —CH₂CH₂CONHR⁸, wherein R⁸ is an unsubstituted, unbranched C₁₁ alkyl chain. In certain embodiments, R³ is —CH₂CH₂CONHR⁸, wherein R⁸ is an unsubstituted, unbranched C₁₂ alkyl chain. In certain embodiments, R³ is —CH₂CH₂CONHR⁸, wherein R⁸ is an unsubstituted, unbranched C₁₃ alkyl chain. In certain embodiments, R³ is —CH₂CH₂CONHR⁸, wherein R⁸ is an unsubstituted, unbranched C₁₄ alkyl chain. In certain embodiments, R³ is —CH₂CH₂CONHR⁸, wherein R⁸ is an unsubstituted, unbranched C₁₅ alkyl chain. In certain embodiments, R³ is —CH₂CH₂CONHR⁸, wherein R⁸ is an unsubstituted, unbranched C₁₆ alkyl chain. In certain embodiments, R³ is —CH₂CH₂CONHR⁸, wherein R⁸ is an unsubstituted, unbranched C₁₇ alkyl chain. In certain embodiments, R³ is —CH₂CH₂CONHR⁸, wherein R⁸ is an unsubstituted, unbranched C₁₈ alkyl chain. In certain embodiments, R³ is —CH₂CH₂CONHR⁸, wherein R⁸ is an unsubstituted, unbranched C₁₉ alkyl chain. In certain embodiments, R³ is —CH₂CH₂CONHR⁸, wherein R⁸ is an unsubstituted, unbranched C₂₀ alkyl chain.

In certain embodiments, R⁴ is hydrogen. In other embodiments, R⁴ is C₁-C₆ alkyl or C₃-C₇ cycloalkyl. In certain embodiments, R⁴ is taken together with R³ and the intervening atoms to form a ring. In certain embodiments, the ring formed by R³, R⁴, and the intervening atoms is pyrrolidine, piperidine, morpholine, or homopiperidine. In certain embodiments, R⁴ is —(CH₂)ₙOR^{A} or —(CH₂)ₙN(R^{A})₂. In certain embodiments, R⁴ is —(CH₂)ₙOR^{A}. In certain embodiments, R⁴ is —(CH₂)₂OH. In certain embodiments, R⁴ is Y. In certain embodiments, R⁴ is —(CH₂)ₙN(Y)₂ or —(CH₂)ₙNR^{A}Y. In other embodiments, R⁴ is —CH₂CH₂CO₂R⁸ or —CH₂CH₂CONHR⁸. In certain embodiments, R⁴ is —CH₂CH₂CO₂R⁸. In certain embodiments, R⁴ is —CH₂CH₂CONHR⁸. In certain embodiments, R⁸ is an unsubstituted, straight chain alkyl group with at least 6 carbons. In certain embodiments, R⁸ is an unsubstituted, straight chain alkyl group, C₆-C₃₀. In certain embodiments, R⁸ is an unsubstituted, straight chain alkyl group, preferably C₉-C₂₀. In certain embodiments, R⁴ is —CH₂CH₂CO₂R⁸, wherein R⁸ is an unsubstituted, unbranched C₉ alkyl chain. In certain embodiments, R⁴ is —CH₂CH₂CO₂R⁸, wherein R⁸ is an unsubstituted, unbranched C₁₀ alkyl chain. In certain embodiments, R⁴ is —CH₂CH₂CO₂R⁸, wherein R⁸ is an unsubstituted, unbranched C₁₁ alkyl chain. In certain embodiments, R⁴ is —CH₂CH₂CO₂R⁸, wherein R⁸ is an unsubstituted, unbranched C₁₂ alkyl chain. In certain embodiments, R⁴ is —CH₂CH₂CO₂R⁸, wherein R⁸ is an unsubstituted, unbranched C₁₃ alkyl chain. In certain embodiments, R⁴ is —CH₂CH₂CO₂R⁸, wherein R⁸ is an unsubstituted, unbranched C₁₄ alkyl chain. In certain embodiments, R⁴ is —CH₂CH₂CO₂R⁸, wherein R⁸ is an unsubstituted, unbranched C₁₅ alkyl chain. In certain embodiments, R⁴ is —CH₂CH₂CO₂R⁸, wherein R⁸ is an unsubstituted, unbranched C₁₆ alkyl chain. In certain embodiments, R⁴ is —CH₂CH₂CO₂R⁸, wherein R⁸ is an unsubstituted, unbranched C₁₇ alkyl chain. In certain embodiments, R⁴ is —CH₂CH₂CO₂R⁸, wherein R⁸ is an unsubstituted, unbranched C₁₈ alkyl chain. In certain embodiments, R⁴ is —CH₂CH₂CO₂R⁸, wherein R⁸ is an unsubstituted, unbranched C₁₉ alkyl chain. In certain embodiments, R⁴ is —CH₂CH₂CO₂R⁸, wherein R⁸ is an unsubstituted, unbranched C₂₀ alkyl chain. In certain embodiments, R⁴ is —CH₂CH₂CONHR⁸, wherein R⁸ is an unsubstituted, unbranched C₉ alkyl chain. In certain embodiments, R⁴ is —CH₂CH₂CONHR⁸, wherein R⁸ is an unsubstituted, unbranched C₁₀ alkyl chain. In certain embodiments, R⁴ is —CH₂CH₂CONHR⁸, wherein R⁸ is an unsubstituted, unbranched C₁₁ alkyl chain. In certain embodiments, R⁴ is —CH₂CH₂CONHR⁸, wherein R⁸ is an unsubstituted, unbranched C₁₂ alkyl chain. In certain embodiments, R⁴ is —CH₂CH₂CONHR⁸, wherein R⁸ is an unsubstituted, unbranched C₁₃ alkyl chain. In certain embodiments, R⁴ is —CH₂CH₂CONHR⁸, wherein R⁸ is an unsubstituted, unbranched C₁₄ alkyl chain. In certain embodiments, R⁴ is —CH₂CH₂CONHR⁸, wherein R⁸ is an unsubstituted, unbranched C₁₅ alkyl chain. In certain embodiments, R⁴ is —CH₂CH₂CONHR⁸, wherein R⁸ is an unsubstituted, unbranched C₁₆ alkyl chain. In certain embodiments, R⁴ is —CH₂CH₂CONHR⁸, wherein R⁸ is an unsubstituted, unbranched C₁₇ alkyl chain. In certain embodiments, R⁴ is —CH₂CH₂CONHR⁸, wherein R⁸ is an unsubstituted, unbranched C₁₈ alkyl chain. In certain embodiments, R⁴ is —CH₂CH₂CONHR⁸, wherein R⁸ is an unsubstituted, unbranched C₁₉ alkyl chain. In certain embodiments, R⁴ is —CH₂CH₂CONHR⁸, wherein R⁸ is an unsubstituted, unbranched C₂₀ alkyl chain.

In certain embodiments, R⁵ is hydrogen. In certain embodiments, R⁵ is methyl. R⁶ is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N₃; —N(R_{A})₂; —NHC(═O)R_{A}; —NR_{A}C(═O)N(R_{A})₂; —OC(═O)OR_{A}; —OC(═O)R_{A}; —OC(═O)N(R_{A})₂; —NR_{A}C(═O)OR_{A}; and —C(R_{A})₃; wherein each occurrence of R_{A} is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; and salts thereof.

In certain embodiments, R⁶ is a cyclic or acyclic, substituted or unsubstituted, branched or unbranched aliphatic moiety. In other embodiments, R⁶ is a cyclic or acyclic, substituted or unsubstituted, branched or unbranched heteroaliphatic moiety. In certain embodiments, R⁶ is a polyethylene glycol moiety. In certain embodiments, R⁶ is an aliphatic moiety substituted with one or more hydroxyl groups. In other embodiments, R⁶ is an aliphatic moiety substituted with one or more amino, alkylamino, or dialkylamino groups. In certain embodiments, R⁶ is a heteroaliphatic moiety. In certain embodiments, R⁶ is cyclic aliphatic, preferably a monocylic ring system with a 5- or 6-membered ring. In other embodiments, R⁶ is aryl or heteroaryl, preferably a monocyclic ring system with a 5- or 6-membered ring. In other embodiments, R⁶ is an imidazole moiety. In certain embodiments, R⁶ is —OR_{A}. In certain embodiments, R⁶ is —OH.

In certain embodiments, R⁷ is hydrogen. In certain embodiments, R⁷ is Y. In certain embodiments, R⁷ is —CH₂CH₂CO₂R⁸ or —CH₂CH₂CONHR⁸. In certain embodiments, R⁷ is —CH₂CH₂CO₂R⁸. In certain embodiments, R⁷ is —CH₂CH₂CONHR⁸. In certain embodiments, R⁸ is an unsubstituted, straight chain alkyl group with at least 6 carbons. In certain embodiments, R⁸ is an unsubstituted, straight chain alkyl group, C₆-C₃₀. In certain embodiments, R⁸ is an unsubstituted, straight chain alkyl group, preferably C₉-C₂₀. In certain embodiments, R⁷ is —CH₂CH₂CO₂R⁸, wherein R⁸ is an unsubstituted, unbranched C₉ alkyl chain. In certain embodiments, R⁷ is —CH₂CH₂CO₂R⁸, wherein R⁸ is an unsubstituted, unbranched C₁₀ alkyl chain. In certain embodiments, R⁷ is —CH₂CH₂CO₂R⁸, wherein R⁸ is an unsubstituted, unbranched C₁₁ alkyl chain. In certain embodiments, R⁷ is —CH₂CH₂CO₂R⁸, wherein R⁸ is an unsubstituted, unbranched C₁₂ alkyl chain. In certain embodiments, R⁷ is —CH₂CH₂CO₂R⁸, wherein R⁸ is an unsubstituted, unbranched C₁₃ alkyl chain. In certain embodiments, R⁷ is —CH₂CH₂CO₂R⁸, wherein R⁸ is an unsubstituted, unbranched C₁₄ alkyl chain. In certain embodiments, R⁷ is —CH₂CH₂CO₂R⁸, wherein R⁸ is an unsubstituted, unbranched C₁₅ alkyl chain. In certain embodiments, R⁷ is —CH₂CH₂CO₂R⁸, wherein R⁸ is an unsubstituted, unbranched C₁₆ alkyl chain. In certain embodiments, R⁷ is —CH₂CH₂CO₂R⁸, wherein R⁸ is an unsubstituted, unbranched C₁₇ alkyl chain. In certain embodiments, R⁷ is —CH₂CH₂CO₂R⁸, wherein R⁸ is an unsubstituted, unbranched C₁₈ alkyl chain. In certain embodiments, R⁷ is —CH₂CH₂CO₂R⁸, wherein R⁸ is an unsubstituted, unbranched C₁₉ alkyl chain. In certain embodiments, R⁷ is —CH₂CH₂CO₂R⁸, wherein R⁸ is an unsubstituted, unbranched C₂₀ alkyl chain. In certain embodiments, R⁷ is —CH₂CH₂CONHR⁸, wherein R⁸ is an unsubstituted, unbranched C₉ alkyl chain. In certain embodiments, R⁷ is —CH₂CH₂CONHR⁸, wherein R⁸ is an unsubstituted, unbranched C₁₀ alkyl chain. In certain embodiments, R⁷ is —CH₂CH₂CONHR⁸, wherein R⁸ is an unsubstituted, unbranched C₁₁ alkyl chain. In certain embodiments, R⁷ is —CH₂CH₂CONHR⁸, wherein R⁸ is an unsubstituted, unbranched C₁₂ alkyl chain. In certain embodiments, R⁷ is —CH₂CH₂CONHR⁸, wherein R⁸ is an unsubstituted, unbranched C₁₃ alkyl chain. In certain embodiments, R⁷ is —CH₂CH₂CONHR⁸, wherein R⁸ is an unsubstituted, unbranched C₁₄ alkyl chain. In certain embodiments, R⁷ is —CH₂CH₂CONHR⁸, wherein R⁸ is an unsubstituted, unbranched C₁₅ alkyl chain. In certain embodiments, R⁷ is —CH₂CH₂CONHR⁸, wherein R⁸ is an unsubstituted, unbranched C₁₆ alkyl chain. In certain embodiments, R⁷ is —CH₂CH₂CONHR⁸, wherein R⁸ is an unsubstituted, unbranched C₁₇ alkyl chain. In certain embodiments, R⁷ is —CH₂CH₂CONHR⁸, wherein R⁸ is an unsubstituted, unbranched C₁₈ alkyl chain. In certain embodiments, R⁷ is —CH₂CH₂CONHR⁸, wherein R⁸ is an unsubstituted, unbranched C₁₉ alkyl chain. In certain embodiments, R⁷ is —CH₂CH₂CONHR⁸, wherein R⁸ is an unsubstituted, unbranched C₂₀ alkyl chain.

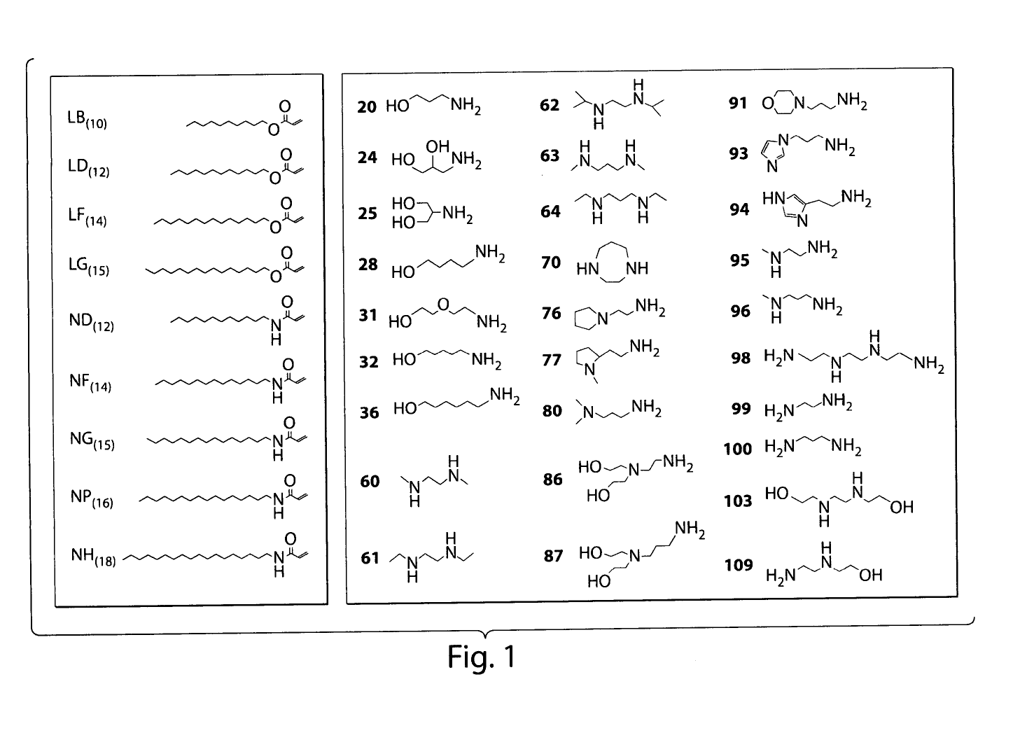

In certain embodiments, the lipidoids are prepared from the amines 61, 62, 64, 76, 80, 86, 87, 91, 95, 96, 99, 100, 103, and 109 shown in FIG. 1.

In certain embodiments, lipidoids that are useful in delivering immunostimulatory polynucleotides are of the formula:

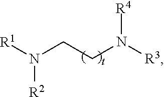

wherein R¹, R², R³, and R⁴ are defined above, and t is 1 or 2.

In certain embodiments, t is 1 as shown in the formula:

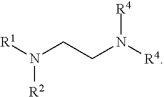

In certain embodiments, t is 2 as shown in the formula:

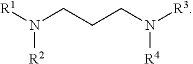

In certain embodiments, the lipidoid is of formula:

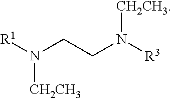

In certain embodiments, the lipidoid is of formula:

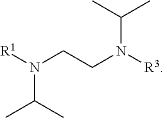

In certain embodiments, the lipidoid is of formula:

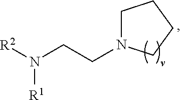

wherein v is 1, 2, or 3. In certain embodiments, v is 1. In certain embodiments, v is 2. In certain embodiments, v is 3.

In certain embodiments, the lipidoid is of formula:

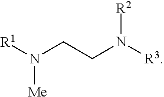

In certain embodiments, the lipidoid is of formula:

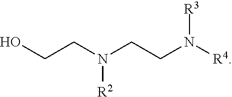

In certain embodiments, the lipidoid is of formula:

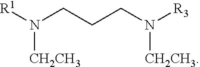

In certain embodiments, the lipidoid is of formula:

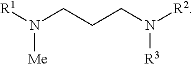

In certain embodiments, the lipidoid useful in delivering immunostimulatory polynucleotides is of formula:

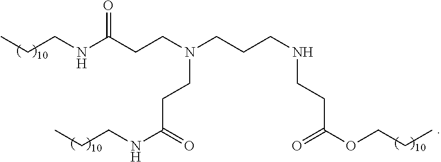

In certain embodiments, the lipidoid useful in delivering immunostimulatory polynucleotides is of formula:

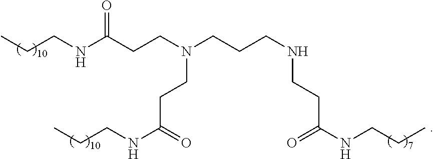

In certain embodiments, the lipidoids useful in delivering immunostimulatory polynucleotides are of the formula:

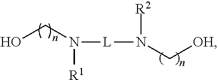

wherein n, R¹, and R² are defined as described herein.

In further embodiments, the lipidoids useful in delivering immunostimulatory polynucleotides are of the formula:

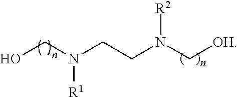

In certain embodiments, n is 2.

In certain embodiments, the lipidoids useful in delivering immunostimulatory polynucleotides are of the formula:

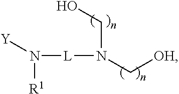

wherein Y, R¹, and n are defined as described herein.

In further embodiments, the lipidoids useful in delivering immunostimulatory polynucleotides are of the formula:

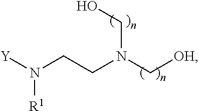

wherein Y, R¹, and n are defined as described herein.

In further embodiments, the lipidoids useful in delivering immunostimulatory polynucleotides are of the formula:

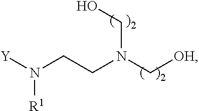

wherein Y and R¹ are defined as described herein.

In certain embodiments, the lipidoids useful in delivering immunostimulatory polynucleotides are of the formula:

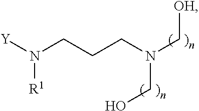

wherein Y, R¹, and n are defined as described herein.

In certain embodiments, the lipidoids useful in delivering immunostimulatory polynucleotides are of the formula:

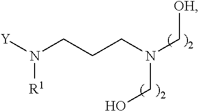

wherein Y and R¹ are defined as described herein.

In certain embodiments, the lipidoids useful in delivering immunostimulatory polynucleotides are of the formula:

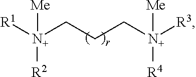

wherein r is 0 or 1. In certain embodiments, r is 0. In certain embodiments, r is 1. In certain embodiments, the lipidoid is of formula:

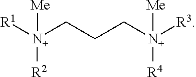

In certain embodiments, the lipidoids useful in delivering immunostimulatory polynucleotides are of the formula:

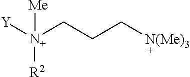

wherein R¹ and Y are defined as described herein.

In certain embodiments, the lipidoids useful in delivering immunostimulatory polynucleotides are of the formula:

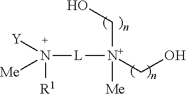

wherein R¹ and Y are defined as described herein. In certain embodiments, n is 2.

In further embodiments, the lipidoids useful in delivering immunostimulatory polynucleotides are of the formulae:

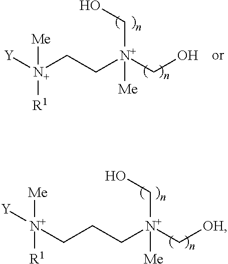

wherein R¹ and Y are defined as described herein, and n is 2, 3, 4, 5, or 6. In certain embodiments, n is 2.

In certain embodiments, the lipidoids useful in delivering immunostimulatory polynucleotides are of the formula:

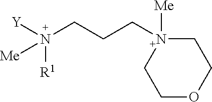

wherein R¹ and Y are defined as described herein.

In certain embodiments, the lipidoids useful in delivering immunostimulatory polynucleotides are of the formula:

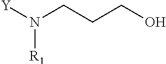

wherein R¹ and Y are defined as described herein.

In certain embodiments, the lipidoids useful in delivering immunostimulatory polynucleotides are of the formula:

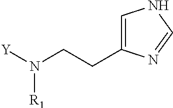

wherein R¹ and Y are defined as described herein.

In certain embodiments, the lipidoids useful in modulating an immune response are of the formulae:

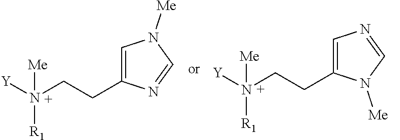

wherein R¹ and Y are defined as described herein.

In certain embodiments, the lipidoids useful in modulating an immune response are of the formula:

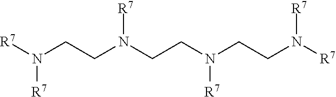

wherein R⁷ is defined as described herein. In certain embodiments, all R⁷ are the same. In certain embodiments, at least one R⁷ is different. In certain embodiments, at least two R⁷ are different. In certain embodiments, at least three R⁷ are different.

In certain embodiments, each instance of Y in the lipidoids described herein is independently selected from the group consisting of:

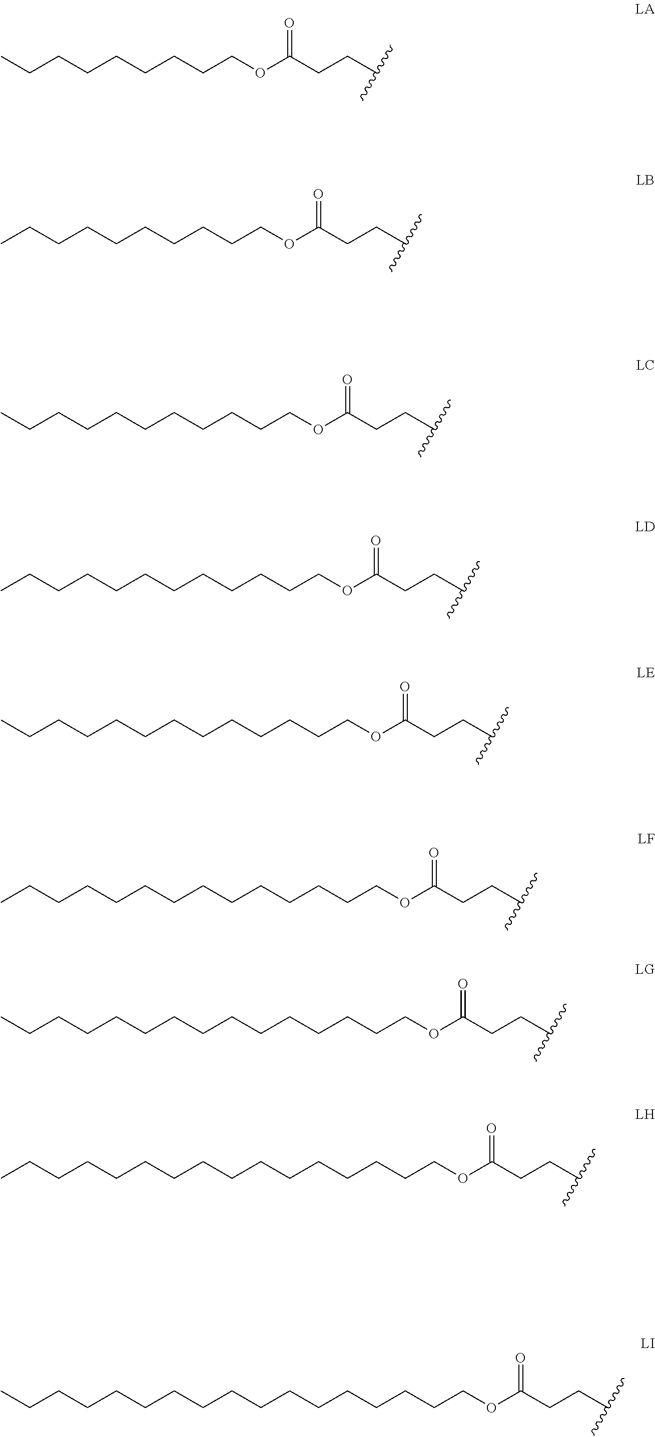

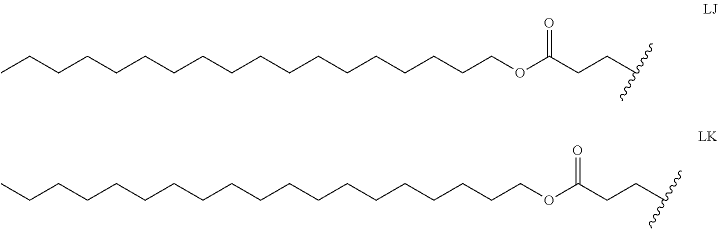

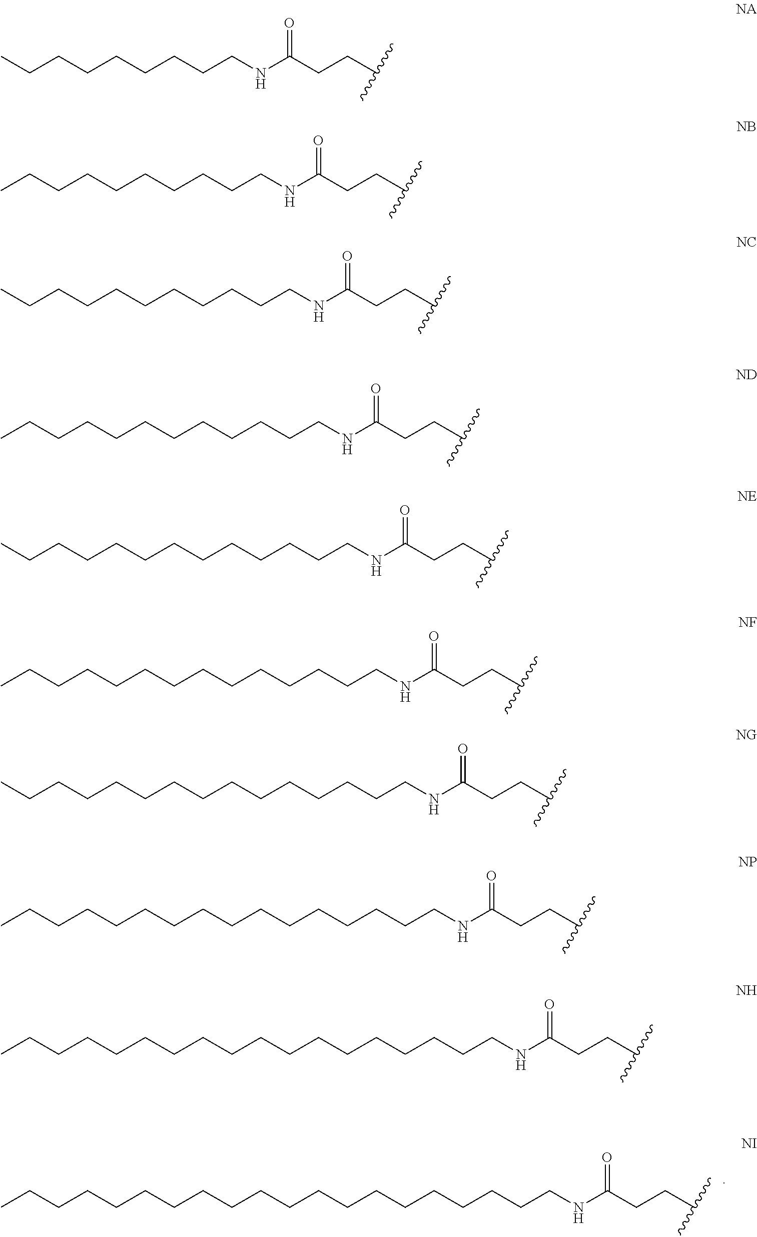

In certain embodiments, the lipidoids are prepared using acrylates LB, LD, LF, LG or acrylamides ND, NF, NG, NP, or NH in FIG. 1. In certain embodiments, the lipidoid is prepared using acrylate LB. In certain embodiments, the lipidoid is prepared using acrylate LD. In certain embodiments, the lipidoid is prepared using acrylate LF. In certain embodiments, the lipidoid is prepared using acrylate LG. In certain embodiments, the lipidoid is prepared using acrylamide ND. In certain embodiments, the lipidoid is prepared using acrylamide NF. In certain embodiments, the lipidoid is prepared using acrylamide NG. In certain embodiments, the lipidoid is prepared using acrylamide NP. In certain embodiments, the lipidoid is prepared using acrylamide NH.

In certain embodiments, the lipidoids are prepared using amines 86, 87, 99, or 100 in FIG. 1. In certain embodiments, the lipidoid is prepared by reacting amine 86, 87, 99, or 100 with an acrylamide or acrylate having a lipid tail from 9-18 carbons long. In certain embodiments, the tail is 12-15 carbons long. In certain embodiments, the acrylamide is NA, NC, ND, NF, NG, or a combination thereof. In certain embodiments, the acrylate is LA, LC, LD, LF, LG, or a combination thereof.

In certain embodiments, the lipidoid of the current invention is prepared by reacting amine 99 with acrylate LF to form lipidoid LF99. In certain embodiments, the lipidoid LF99 is of one of the formulae:

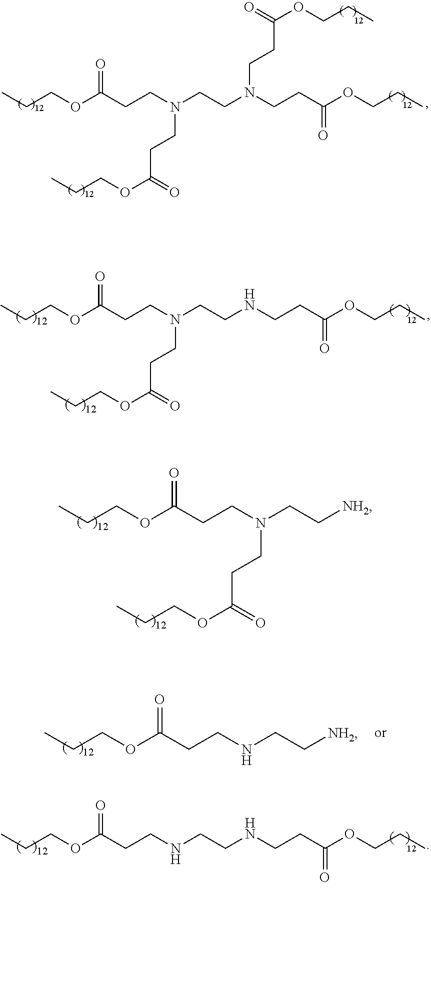

In other embodiments, the lipidoid is a composition of one or more of the above LF99 lipidoids.

In certain embodiments, the lipidoid of the current invention is prepared by reacting amine 99 with acrylamide NF to form lipidoid NF99. In certain embodiments, the lipidoid NF99 is of one of the formulae:

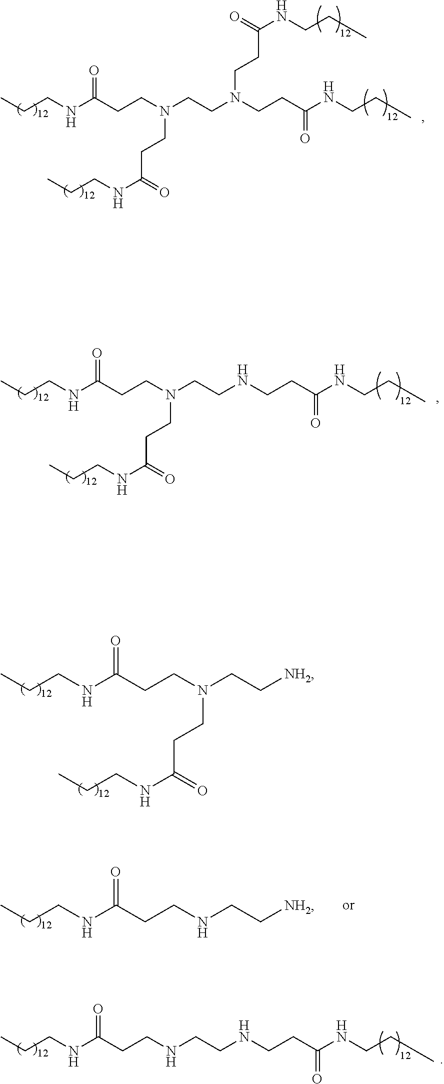

In other embodiments, the lipidoid is a composition of one or more of the above NF99 lipidoids.

In certain embodiments, the lipidoid of the current invention is prepared by reacting amine 99 with acrylamide ND to form lipidoid ND99. In certain embodiments, the lipidoid ND99 is of one of the formulae:

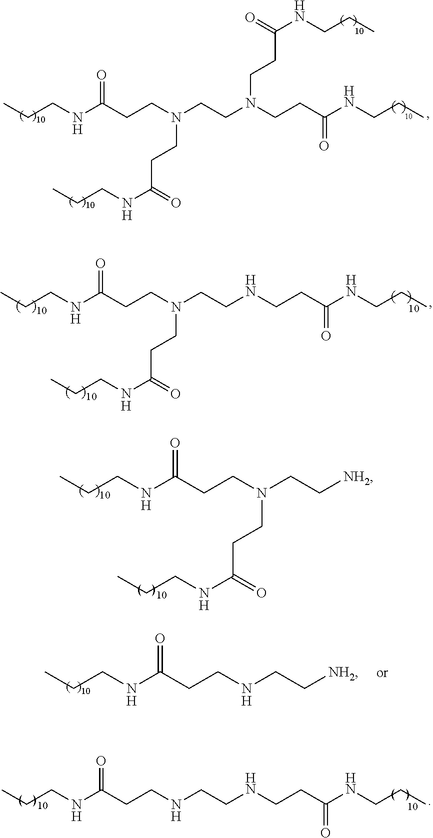

In other embodiments, the lipidoid is a composition of one or more of the above ND99 lipidoids.

In certain embodiments, the lipidoid of the current invention is prepared by reacting amine 61 with acrylamide NF to form lipidoid NF61. In certain embodiments, the lipidoid NF61 is of one of the formulae:

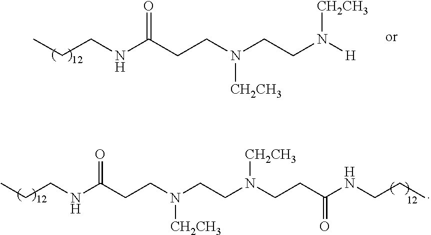

In other embodiments, the lipidoid is a composition of one or more of the above NF61 lipidoids.

In certain embodiments, the lipidoid of the current invention is prepared by reacting amine 61 with acrylamide NG to form lipidoid NG61. In certain embodiments, the lipidoid NG61 is of one of the formulae:

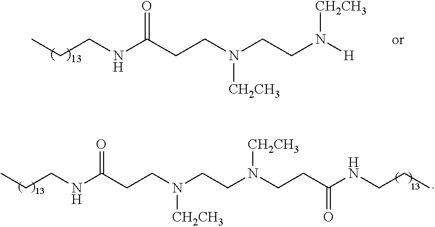

In other embodiments, the lipidoid is a composition of one or more of the above NG61 lipidoids.

In certain embodiments, the lipidoid of the current invention is prepared by reacting amine 62 with acrylamide NP to form lipidoid NP62. In certain embodiments, the lipidoid NP62 is of one of the formulae:

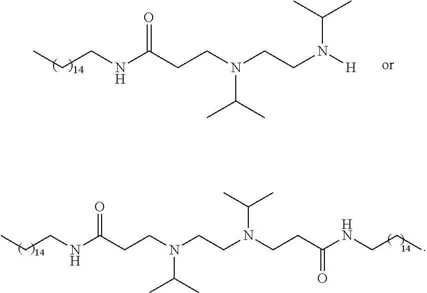

In other embodiments, the lipidoid is a composition of one or more of the above NP62 lipidoids.

In certain embodiments, the lipidoid of the current invention is prepared by reacting amine 76 with acrylate LG to form lipidoid LG76. In certain embodiments, the lipidoid LG76 is of one of the formulae:

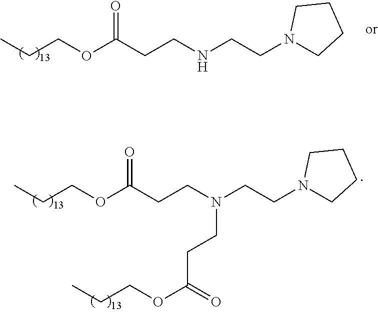

In other embodiments, the lipidoid is a composition of one or more of the above LG76 lipidoids.

In certain embodiments, the lipidoid of the current invention is prepared by reacting amine 95 with acrylamide ND to form lipidoid ND95. In certain embodiments, the lipidoid ND95 is of one of the formulae:

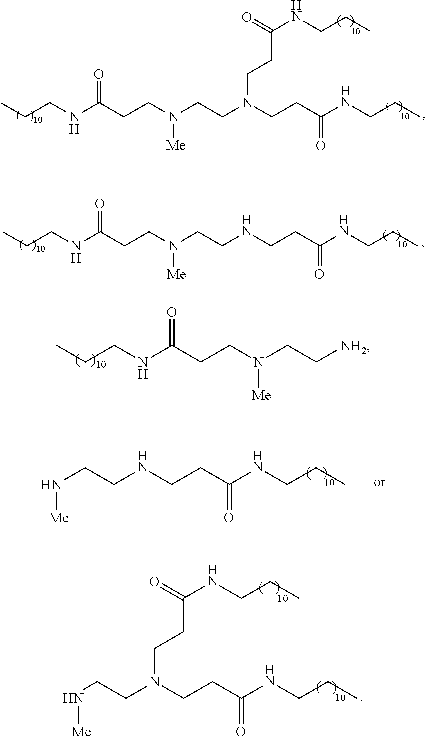

In other embodiments, the lipidoid is a composition of one or more of the above ND95 lipidoids.

In certain embodiments, the lipidoid of the current invention is prepared by reacting amine 95 with acrylamide NF to form lipidoid NF95. In certain embodiments, the lipidoid NF95 is of one of the formulae:

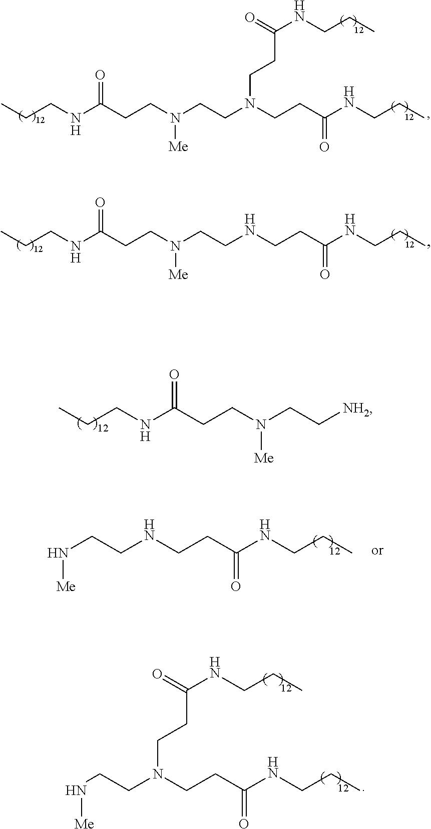

In other embodiments, the lipidoid is a composition of one or more of the above NF95 lipidoids.

In certain embodiments, the lipidoid of the current invention is prepared by reacting amine 109 with acrylate LF to form lipidoid LF109. In certain embodiments, the lipidoid LF109 is of one of the formulae:

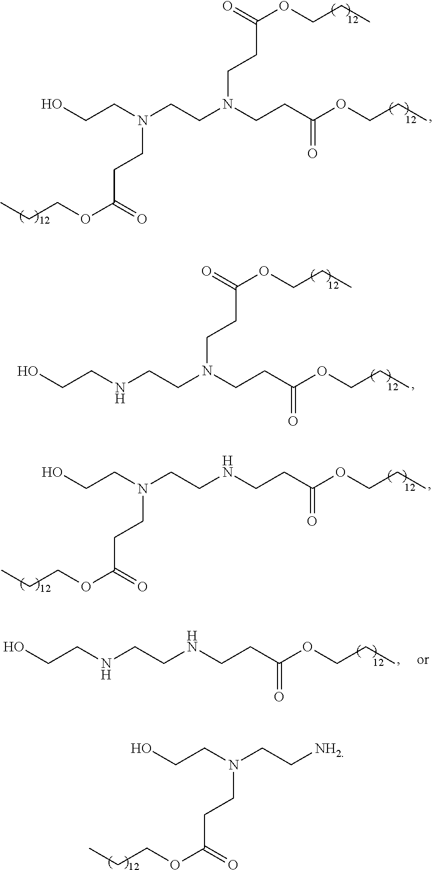

In other embodiments, the lipidoid is a composition of one or more of the above LF109 lipidoids.

In certain embodiments, the lipidoid of the current invention is prepared by reacting amine 109 with acrylamide NF to form lipidoid NF109. In certain embodiments, the lipidoid NF109 is of one of the formulae:

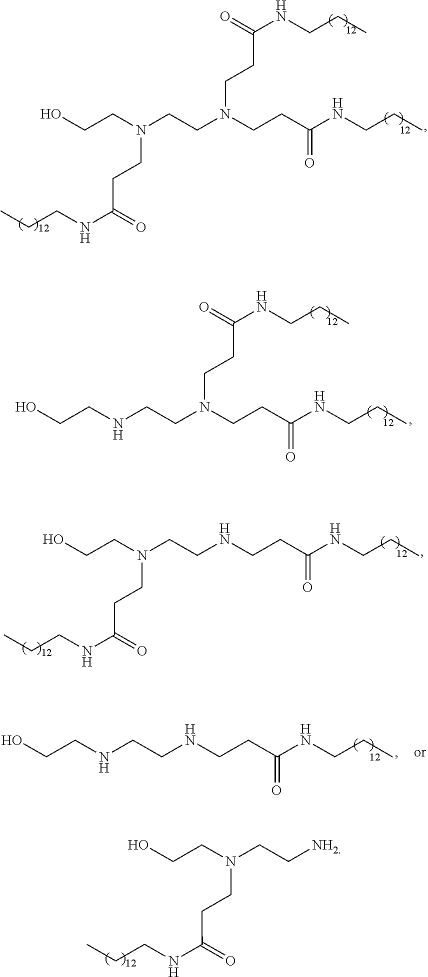

In other embodiments, the lipidoid is a composition of one or more of the above NF109 lipidoids.

In certain embodiments, the lipidoid of the current invention is prepared by reacting amine 109 with acrylamide ND to form lipidoid ND109. In certain embodiments, the lipidoid ND109 is of one of the formulae:

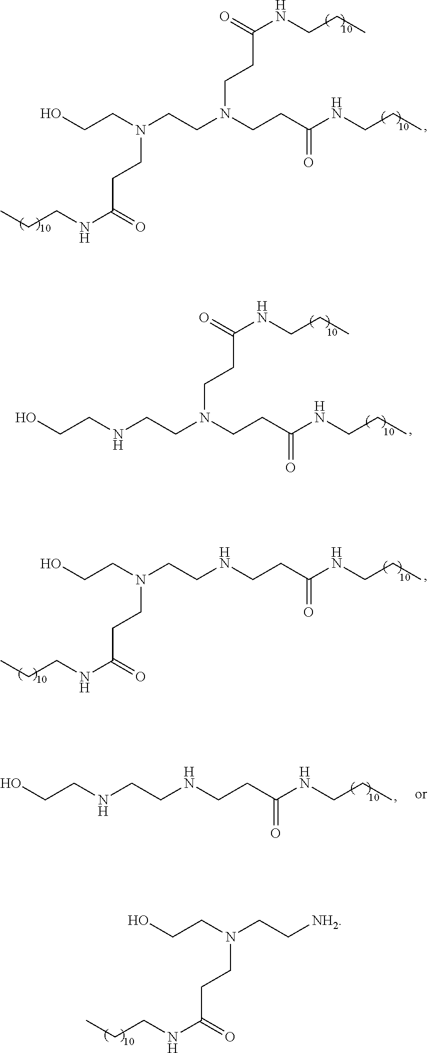

In other embodiments, the lipidoid is a composition of one or more of the above ND109 lipidoids.

In certain embodiments, the lipidoid of the current invention is prepared by reacting amine 100 with acrylate LF to form lipidoid LF100. In certain embodiments, the lipidoid LF100 is of one of the formulae:

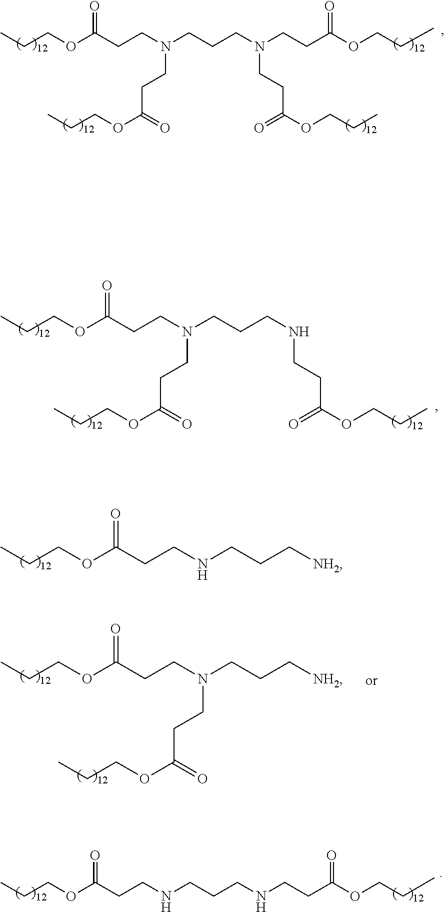

In other embodiments, the lipidoid is a composition of one or more of the above LF100 lipidoids.

In certain embodiments, the lipidoid of the current invention is prepared by reacting amine 100 with acrylamide NF to form lipidoid NF100. In certain embodiments, the lipidoid NF100 is of one of the formulae:

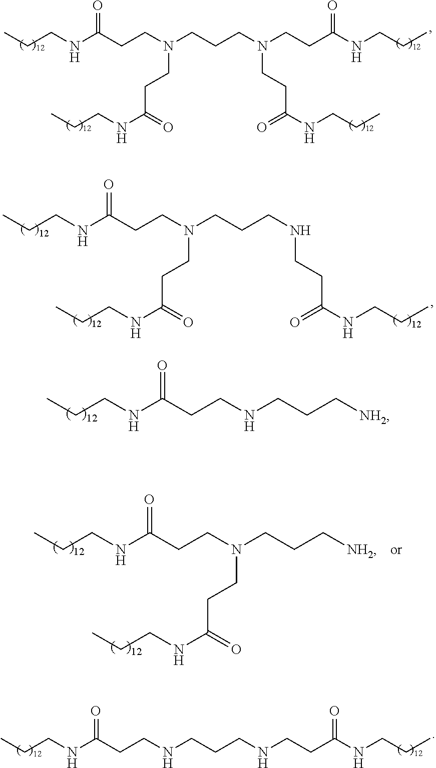

In other embodiments, the lipidoid is a composition of one or more of the above NF100 lipidoids.

In certain embodiments, the lipidoid of the current invention is prepared by reacting amine 100 with acrylamide ND to form lipidoid ND100. In certain embodiments, the lipidoid ND100 is of one of the formulae:

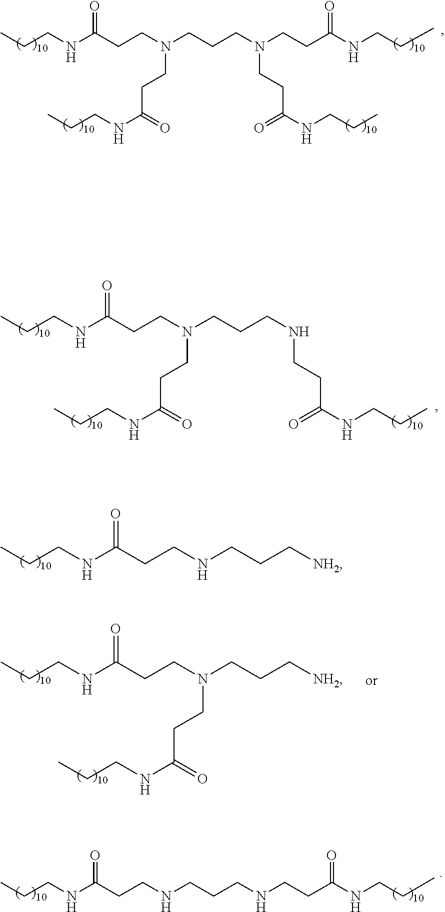

In other embodiments, the lipidoid is a composition of one or more of the above ND100 lipidoids.

In certain embodiments, the lipidoid of the current invention is prepared by reacting amine 100 with acrylamide NG to form lipidoid NG100. In certain embodiments, the lipidoid NG100 is of one of the formulae:

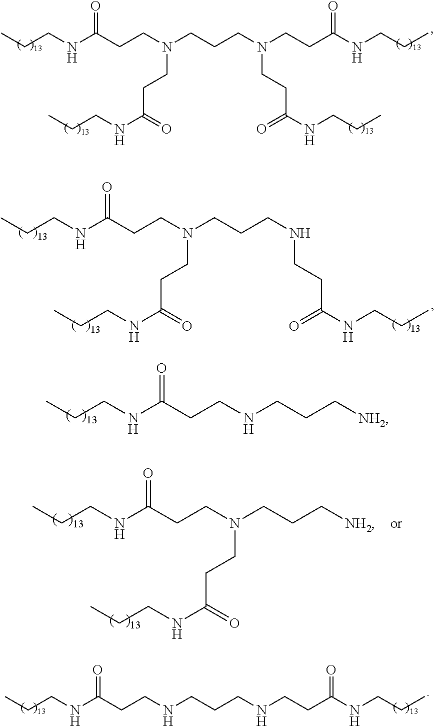

In other embodiments, the lipidoid is a composition of one or more of the above NG100 lipidoids.

In certain embodiments, the lipidoid of the current invention is prepared by reacting amine 64 with acrylamide NG to form lipidoid NG64. In certain embodiments, the lipidoid NG64 is of one of the formulae:

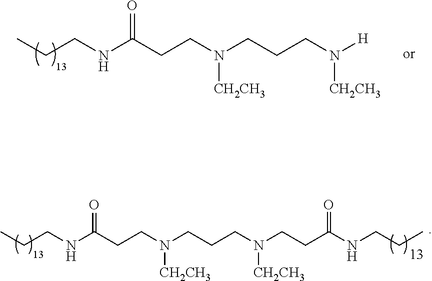

In other embodiments, the lipidoid is a composition of one or more of the above NG64 lipidoids.

In certain embodiments, the lipidoid of the current invention is prepared by reacting amine 96 with acrylamide NF to form lipidoid NF96. In certain embodiments, the lipidoid NF96 is of one of the formulae:

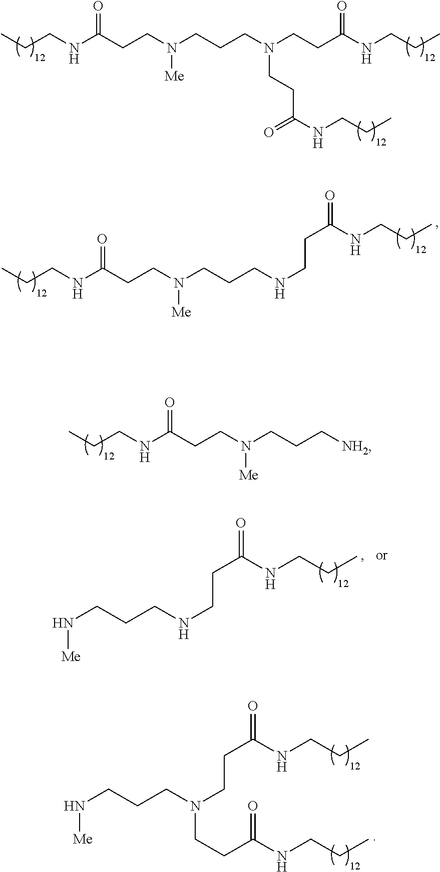

In other embodiments, the lipidoid is a composition of one or more of the above NF96 lipidoids.

In certain embodiments, the lipidoid of the current invention is prepared by reacting amine 96 with acrylamide NG to form lipidoid NG96. In certain embodiments, the lipidoid NG96 is of one of the formulae:

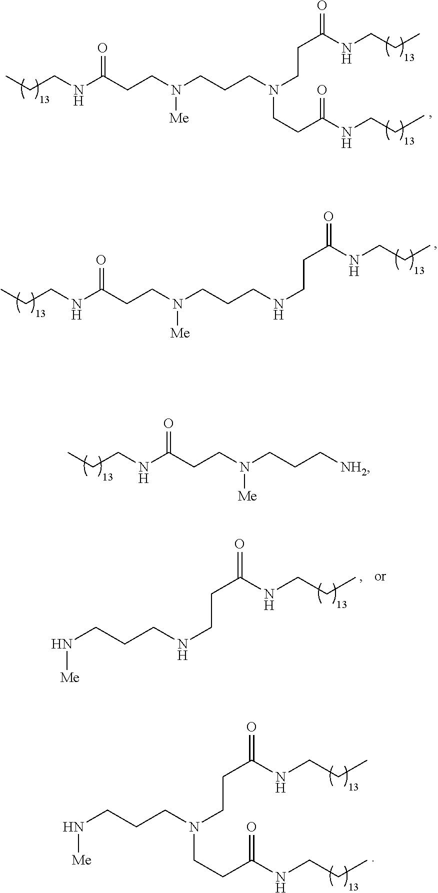

In other embodiments, the lipidoid is a composition of one or more of the above NG96 lipidoids.

In certain embodiments, the lipidoid of the current invention is prepared by reacting amine 96 with acrylamide ND to form lipidoid ND96. In certain embodiments, the lipidoid ND96 is of one of the formulae:

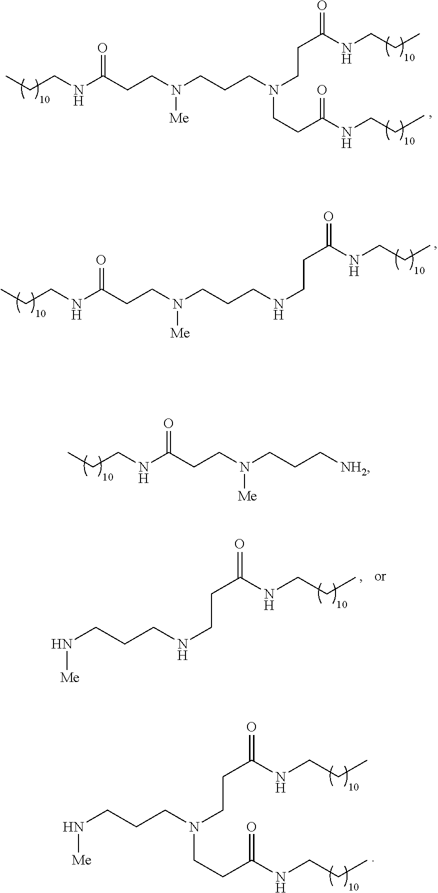

In other embodiments, the lipidoid is a composition of one or more of the above ND96 lipidoids.

In certain embodiments, the lipidoid of the current invention is prepared by reacting amine 96 with acrylate LF to form lipidoid LF96. In certain embodiments, the lipidoid LF96 is of one of the formulae:

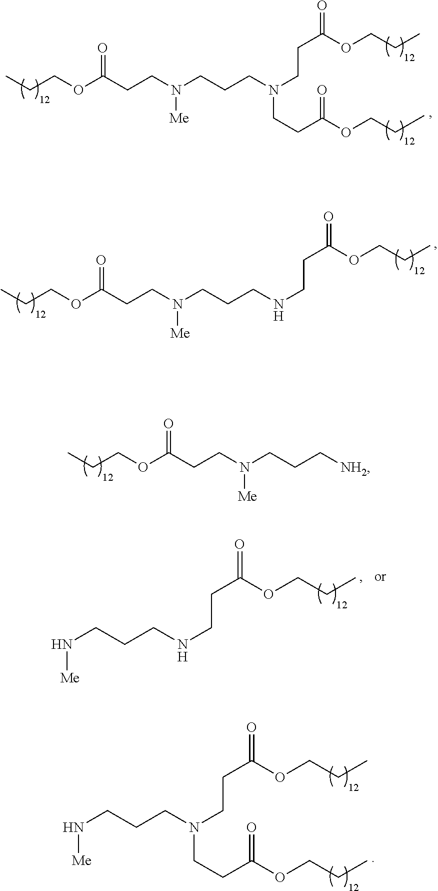

In other embodiments, the lipidoid is a composition of one or more of the above LF96 lipidoids.

In certain embodiments, the lipidoid of the current invention is prepared by reacting amine 96 with acrylate LG to form lipidoid LG96. In certain embodiments, the lipidoid LG96 is of one of the formulae:

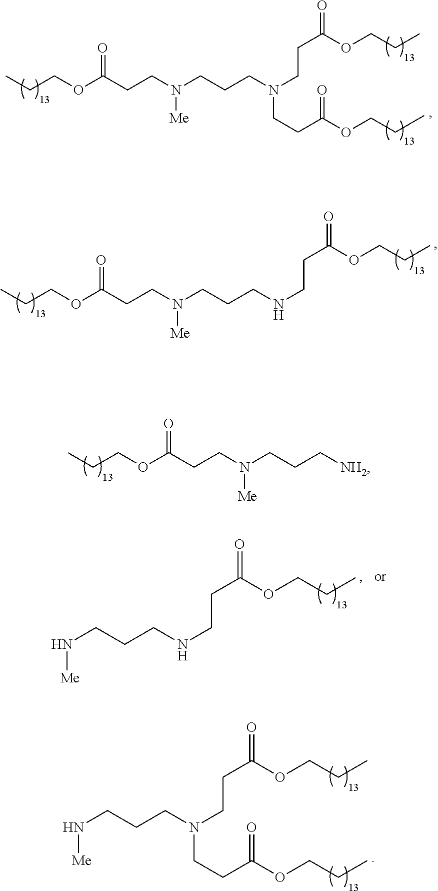

In other embodiments, the lipidoid is a composition of one or more of the above LG96 lipidoids.

In certain embodiments, the lipidoid of the current invention is prepared by reacting amine 103 with acrylamide NG to form lipidoid NG103. In certain embodiments, the lipidoid NG103 is of one of the formulae:

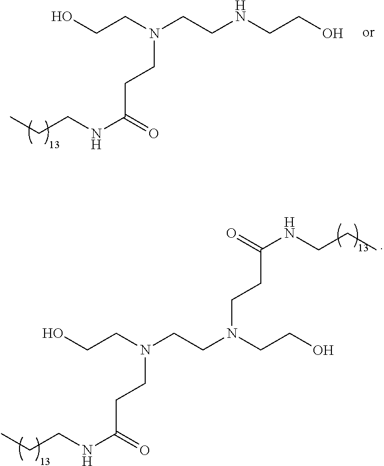

In other embodiments, the lipidoid is a composition of one or more of the above NG103 lipidoids.

In certain embodiments, the lipidoid of the current invention is prepared by reacting amine 103 with acrylamide NF to form lipidoid NF103. In certain embodiments, the lipidoid NF103 is of one of the formulae:

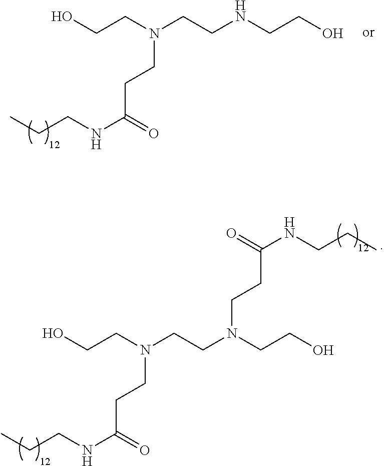

In other embodiments, the lipidoid is a composition of one or more of the above NF103 lipidoids.

In certain embodiments, the lipidoid of the current invention is prepared by reacting amine 103 with acrylamide NP to form lipidoid NP103. In certain embodiments, the lipidoid NP103 is of one of the formulae:

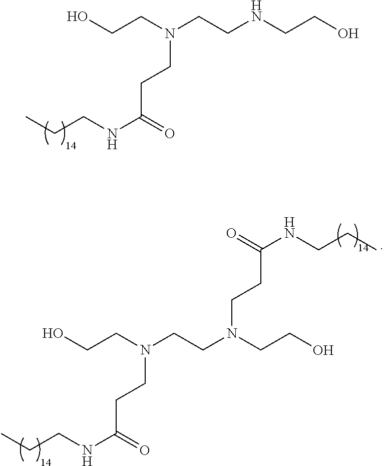

In other embodiments, the lipidoid is a composition of one or more of the above NP103 lipidoids.

In certain embodiments, the lipidoid of the current invention is prepared by reacting amine 86 with acrylamide NF to form lipidoid NF86. In certain embodiments, the lipidoid NF86 is of one of the formulae:

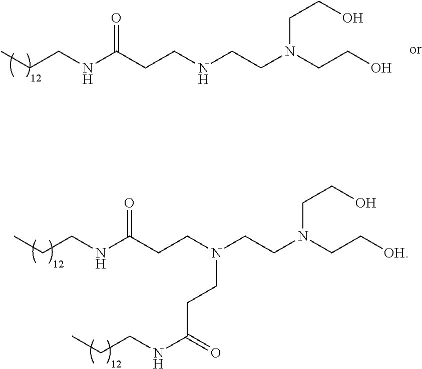

In other embodiments, the lipidoid is a composition of one or more of the above NF86 lipidoids.

In certain embodiments, the lipidoid of the current invention is prepared by reacting amine 86 with acrylamide NG to form lipidoid NG86. In certain embodiments, the lipidoid NG86 is of one of the formulae:

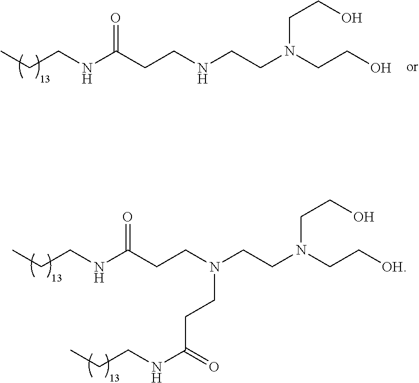

In other embodiments, the lipidoid is a composition of one or more of the above NG86 lipidoids.

In certain embodiments, the lipidoid of the current invention is prepared by reacting amine 86 with acrylamide NP to form lipidoid NP86. In certain embodiments, the lipidoid NP86 is of one of the formulae:

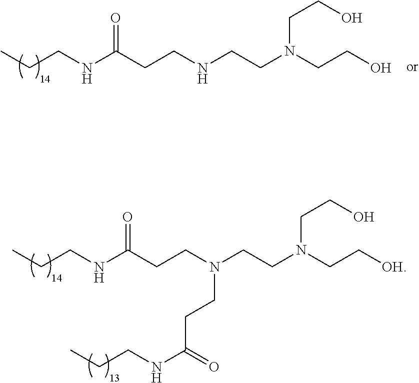

In other embodiments, the lipidoid is a composition of one or more of the above NP86 lipidoids.

In certain embodiments, the lipidoid of the current invention is prepared by reacting amine 87 with acrylamide NF to form lipidoid NF87. In certain embodiments, the lipidoid NF87 is of one of the formulae:

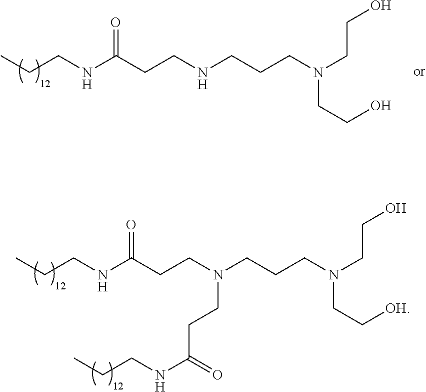

In other embodiments, the lipidoid is a composition of one or more of the above NF87 lipidoids.

In certain embodiments, the lipidoid of the current invention is prepared by reacting amine 87 with acrylamide NG to form lipidoid NG87. In certain embodiments, the lipidoid NG87 is of one of the formulae:

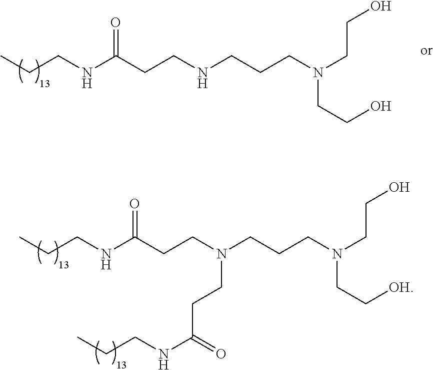

In other embodiments, the lipidoid is a composition of one or more of the above NG87 lipidoids.

In certain embodiments, the lipidoid of the current invention is prepared by reacting amine 87 with acrylamide NP to form lipidoid NP87. In certain embodiments, the lipidoid NP87 is of one of the formulae:

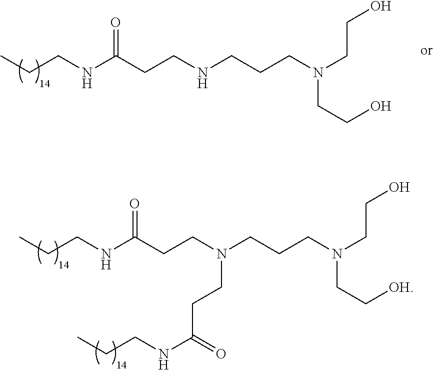

In other embodiments, the lipidoid is a composition of one or more of the above NP87 lipidoids.

In certain embodiments, the lipidoid of the current invention is prepared by reacting amine 20 with acrylamide ND to form lipidoid ND20. In certain embodiments, the lipidoid ND20 is of one of the formulae:

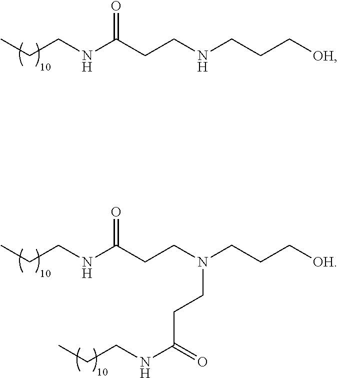

In other embodiments, the lipidoid is a composition of one or more of the above ND20 lipidoids.

In certain embodiments, the lipidoid of the current invention is prepared by reacting amine 98 with acrylamide ND to form lipidoid ND98. In certain embodiments, the lipidoid ND98 is of one of the formulae:

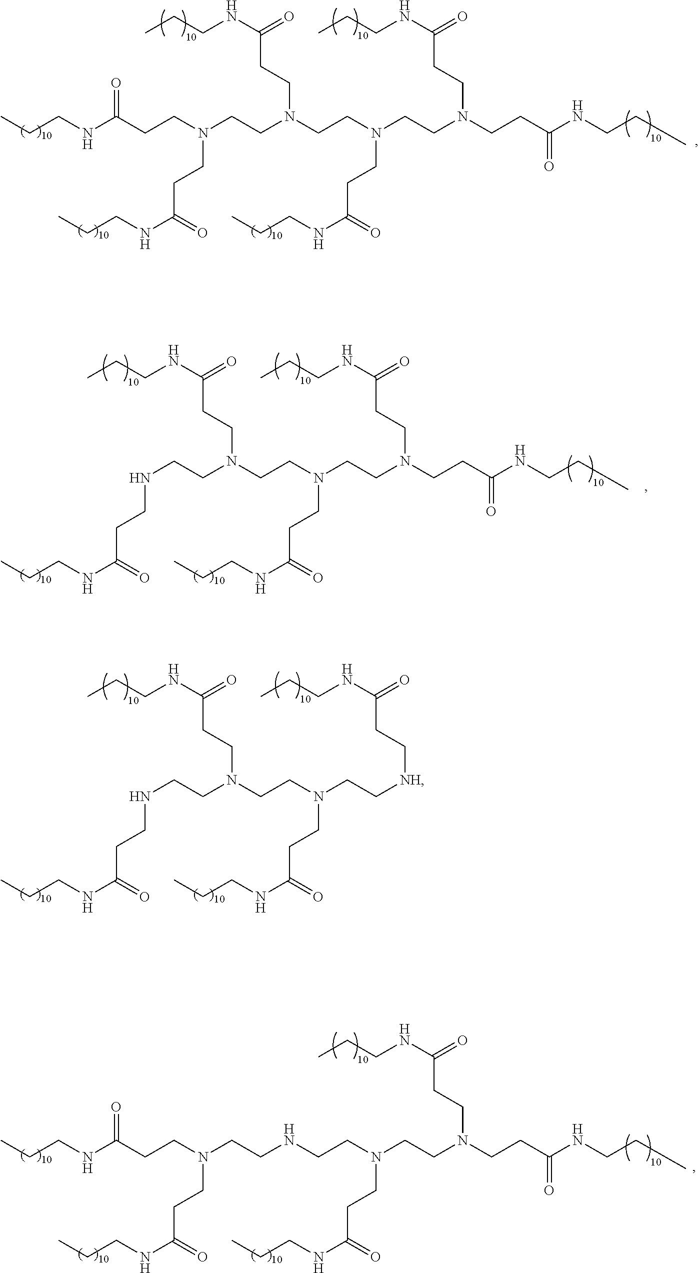

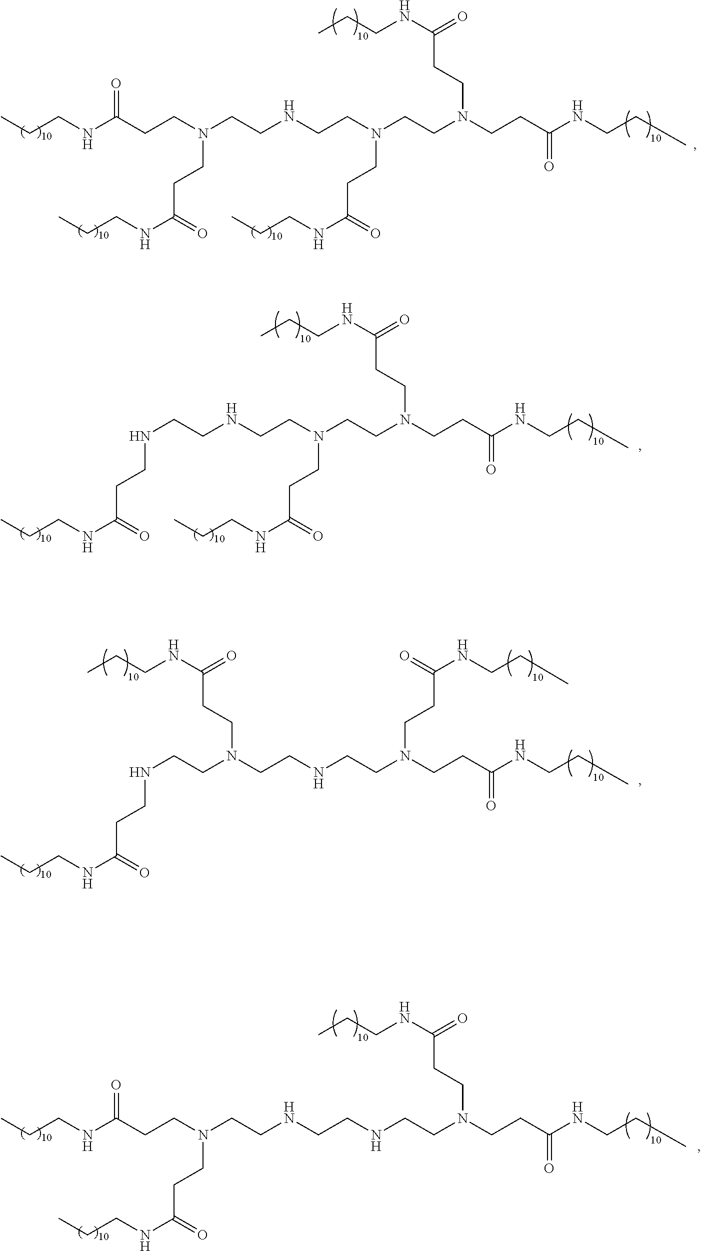

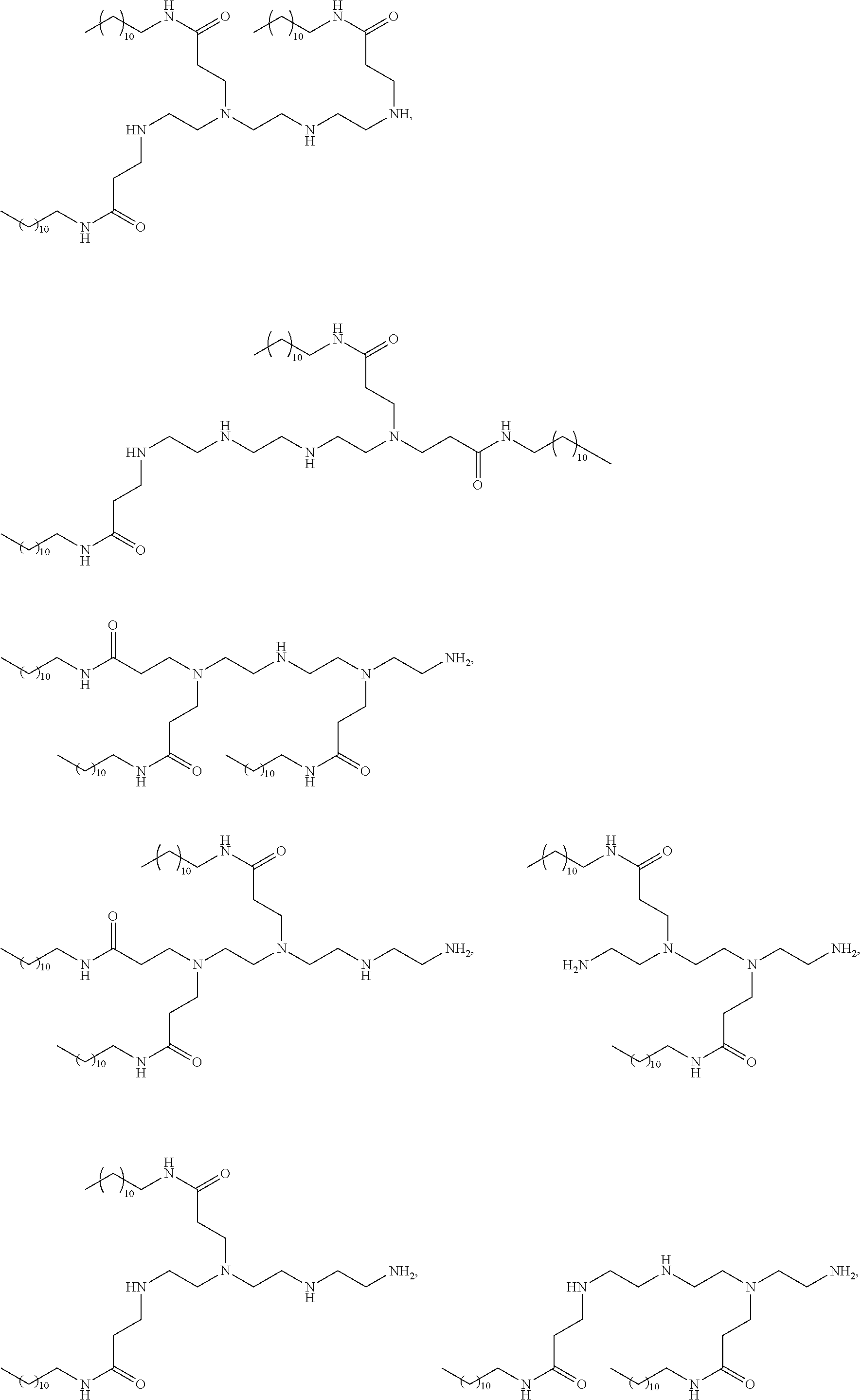

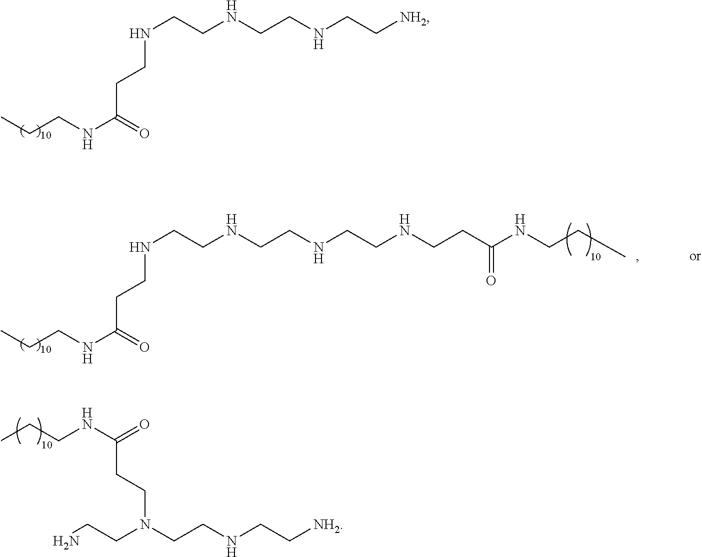

In other embodiments, the lipidoid is a composition of one or more of the above ND98 lipidoids.

In certain embodiments, the lipidoid of the current invention is prepared by reacting amine 63 with acrylate LG to form lipidoid LG63. In certain embodiments, LG63 is further reacted with methyl iodide to give QG63. In certain embodiments, the lipidoid QG63 is of one of the formulae:

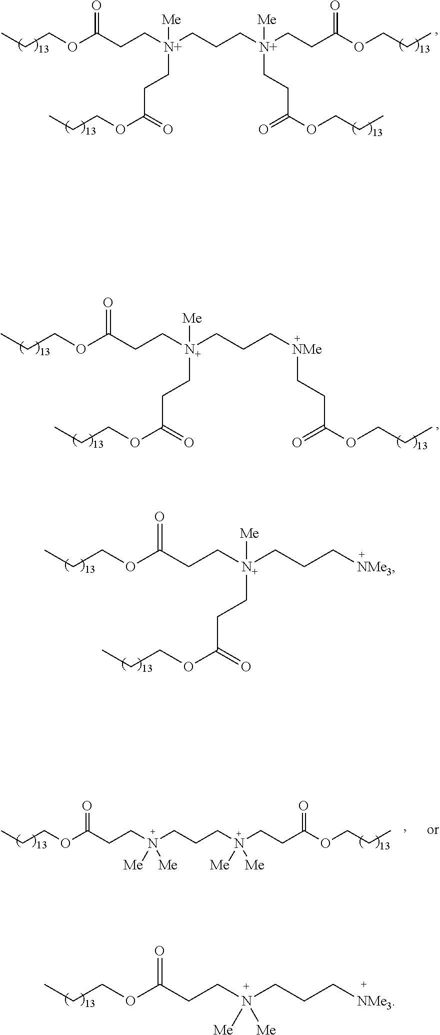

In other embodiments, the lipidoid is a composition of one or more of the above QG63 lipidoids.

In certain embodiments, the lipidoid of the current invention is prepared by reacting amine 80 with acrylate LF to form lipidoid LF80. In certain embodiments, LF80 is further reacted with methyl iodide to give QF80. In certain embodiments, the lipidoid QF80 is of one of the formulae:

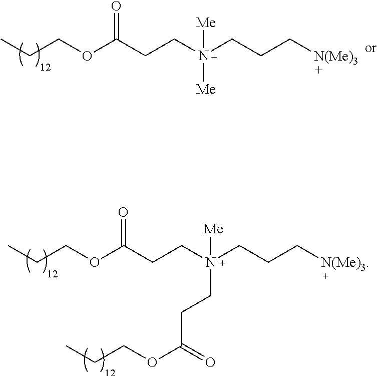

In other embodiments, the lipidoid is a composition of one or more of the above QF80 lipidoids.

In certain embodiments, the lipidoid of the current invention is prepared by reacting amine 86 with acrylate LF to form lipidoid LF86. In certain embodiments, LF86 is further reacted with methyl iodide to give QF86. In certain embodiments, the lipidoid QF86 is of one of the formulae:

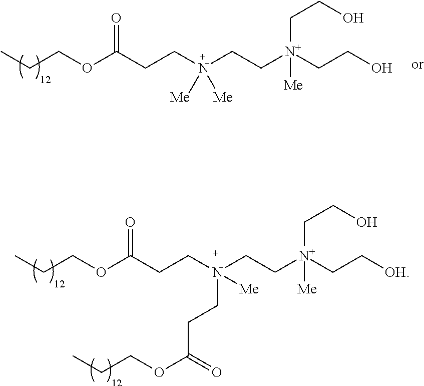

In other embodiments, the lipidoid is a composition of one or more of the above QF86 lipidoids.

In certain embodiments, the lipidoid of the current invention is prepared by reacting amine 87 with acrylate LF to form lipidoid LF87. In certain embodiments, LF87 is further reacted with methyl iodide to give QF87. In certain embodiments, the lipidoid QF87 is of one of the formulae:

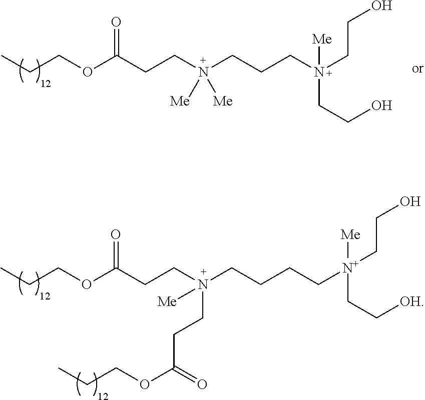

In other embodiments, the lipidoid is a composition of one or more of the above QF87 lipidoids.

In certain embodiments, the lipidoid of the current invention is prepared by reacting amine 87 with acrylate LG to form lipidoid LG87. In certain embodiments, LG87 is further reacted with methyl iodide to give QG87. In certain embodiments, the lipidoid QG87 is of one of the formulae:

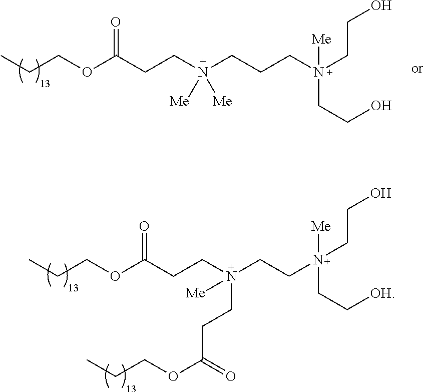

In other embodiments, the lipidoid is a composition of one or more of the above QG87 lipidoids.

In certain embodiments, the lipidoid of the current invention is prepared by reacting amine 91 with acrylate LF to form lipidoid LF91. In certain embodiments, LF91 is further reacted with methyl iodide to give QF91. In certain embodiments, the lipidoid QF91 is of one of the formulae:

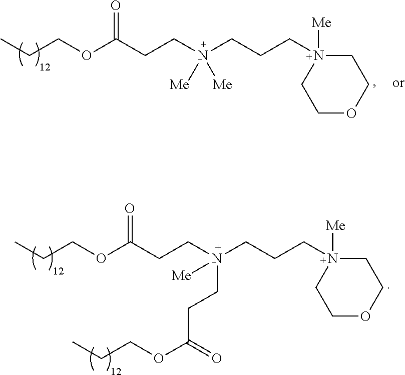

In other embodiments, the lipidoid is a composition of one or more of the above QF91 lipidoids.

In certain embodiments, the lipidoid of the current invention is prepared by reacting amine 94 with acrylate LF to form lipidoid LF94. In certain embodiments, the lipidoid LF94 is of one of the formulae:

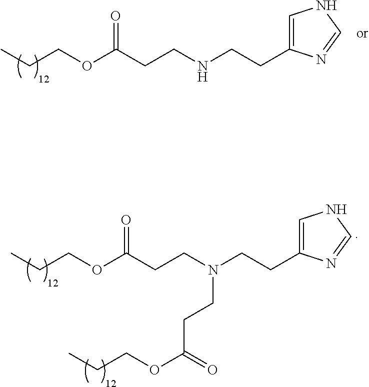

In other embodiments, the lipidoid is a composition of one or more of the above LF94 lipidoids.

In certain embodiments, the lipidoid of the current invention is prepared by reacting amine 94 with acrylate LD to form lipidoid LD94. In certain embodiments, LD94 is further reacted with methyl iodide to give QD94. In certain embodiments, the lipidoid QD94 is of one of the formulae:

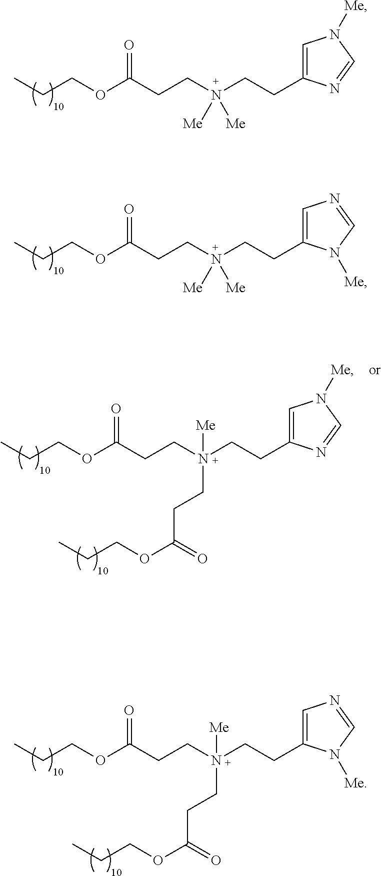

In other embodiments, the lipidoid is a composition of one or more of the above QD94 lipidoids.

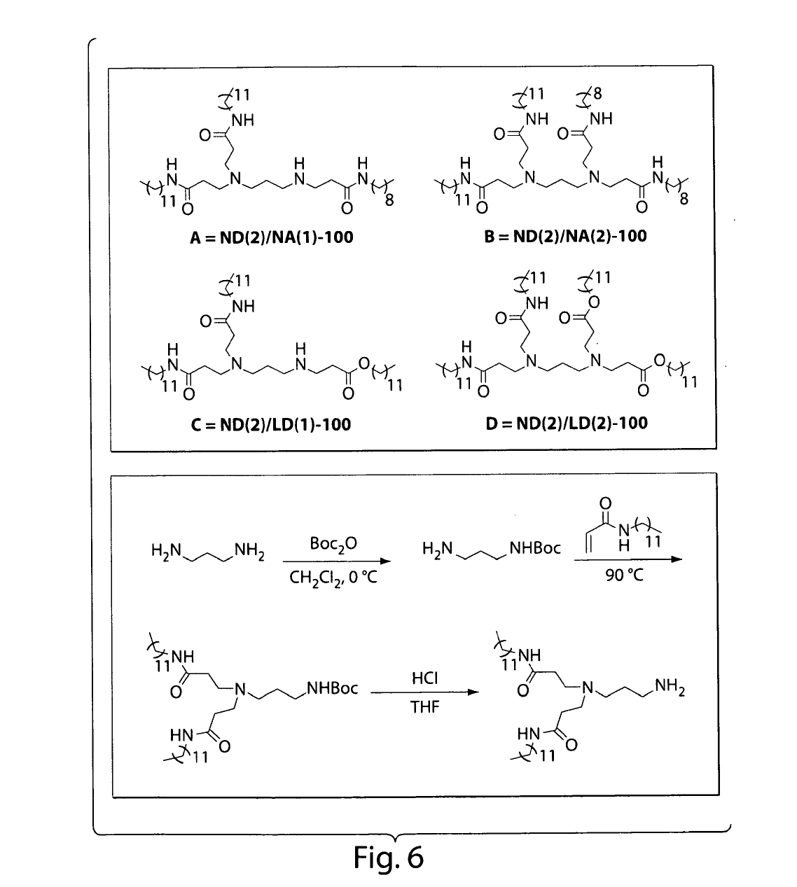

In certain embodiments, the lipidoid of the current invention is prepared by reacting one of the diamine cores from FIG. 1 with a protecting group to protect one of the nitrogens. The free amine group may then be reacted with an acrylate or acrylamide, and the protected amine is deprotected and then reacted with a different acrylate or acrylamide to form a lipidoid with mixed tails (see, e.g., FIG. 6). In certain embodiments, the lipidoid is one of formulae:

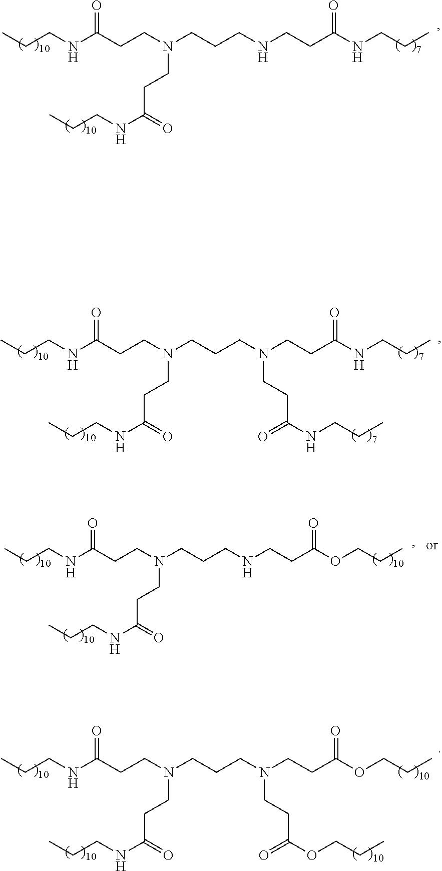

In other embodiments, the lipidoid is a composition of one or more of the above lipidoids.

The present invention also provides novel lipidoids. In certain embodiments, the lipidoid is prepared by reacting amine 76 with an acrylamide or acrylate having a lipid tail from 9-18 carbons long. In certain embodiments, the tail is 12-15 carbons long. In certain embodiments, amine 76 is reacted with acrylate LG to form lipidoid LG96. In certain embodiments, the lipidoid LG76 is of the formulae:

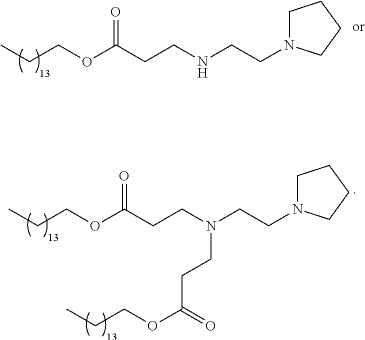

In other embodiments, the lipidoid is a composition of one or more of the above LG96 lipidoids.

In certain embodiments, the lipidoid is prepared by reacting amine 96 with an acrylamide or acrylate having a lipid tail from 9-18 carbons long. In certain embodiments, the tail is 12-15 carbons long. In certain embodiments, amine 96 is reacted with acrylamide ND to form lipidoid ND96. In certain embodiments, the lipidoid ND96 is of the formulae:

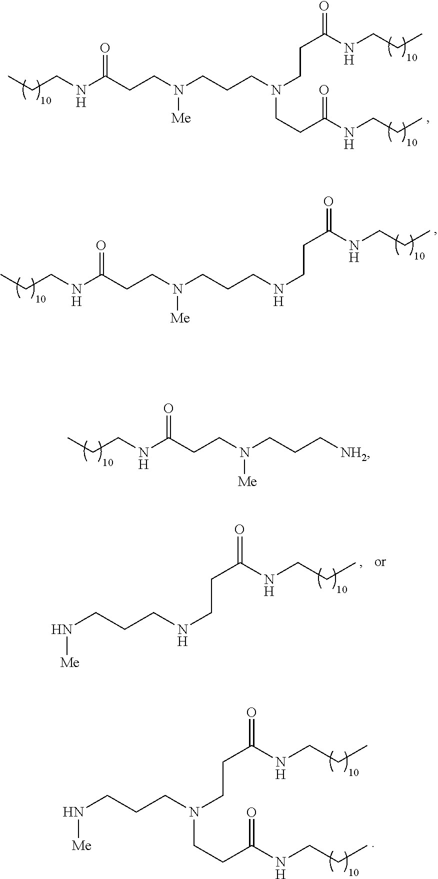

In certain embodiments, amine 96 is reacted with acrylate LF to form lipidoid LF96. In certain embodiments, the lipidoid LF96 is of the formulae:

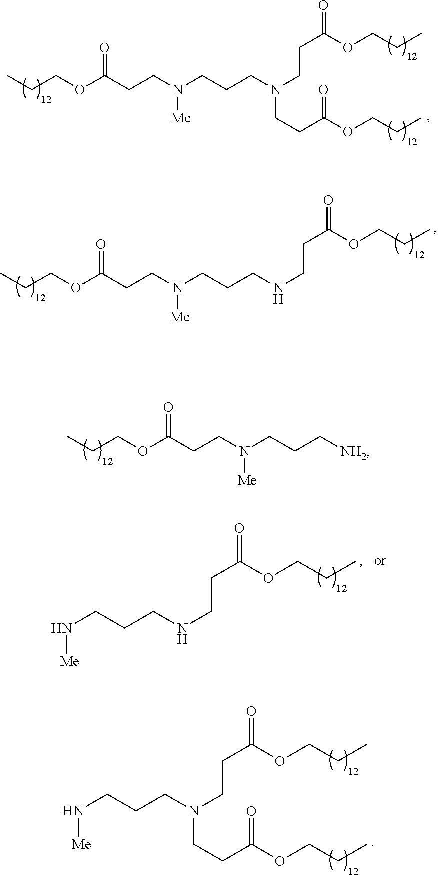

In other embodiments, the lipidoid is a composition of one or more of the above ND96 or LF 96 lipidoids.

In certain embodiments, the lipidoid is prepared by reacting amine 100 with an acrylamide or acrylate having a lipid tail from 9-18 carbons long. In certain embodiments, the tail is 12-15 carbons long. In certain embodiments, amine 100 is reacted with acrylate LF to form lipidoid LF100. In certain embodiments, the lipidoid LF100 is of the formulae:

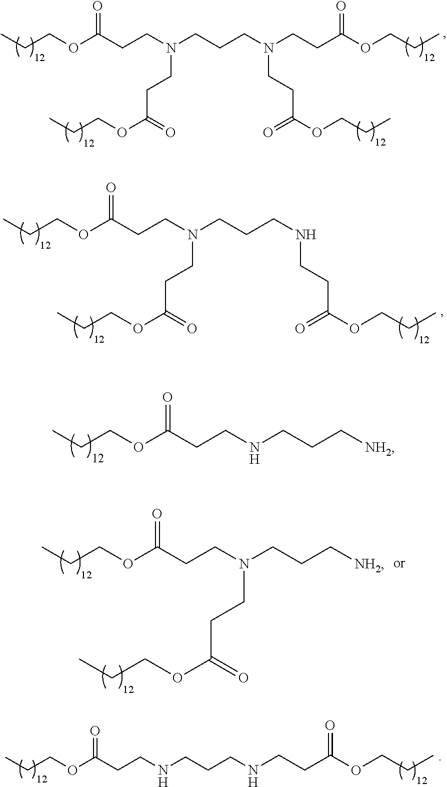

In other embodiments, the lipidoid is a composition of one or more of the above LF100 lipidoids.

In certain embodiments, the lipidoid is prepared by reacting amine 103 with an acrylamide or acrylate having a lipid tail from 9-18 carbons long. In certain embodiments, the tail is 12-15 carbons long. In certain embodiments, amine 103 is reacted with acrylamide NG to form lipidoid NG103. In certain embodiments, the lipidoid NG103 is of the formulae:

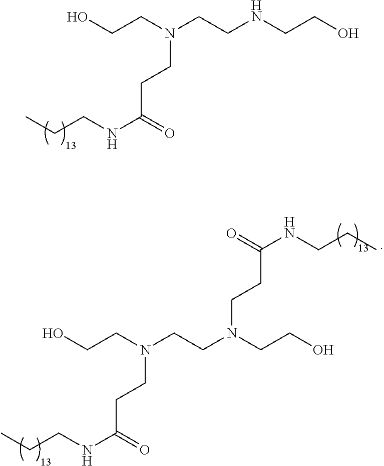

In other embodiments, the lipidoid is a composition of one or more of the above NG 103 lipidoids.

In certain embodiments, the lipidoid is prepared by reacting amine 80 with an acrylamide or acrylate having a lipid tail from 9-18 carbons long. In certain embodiments, the tail is 12-15 carbons long. In certain embodiments, amine 80 is reacted with acrylate LF to form lipidoid LF80. In certain embodiments, LF80 is reacted with methyl iodide to form QF80. In certain embodiments, the lipidoid QF80 is of the formulae:

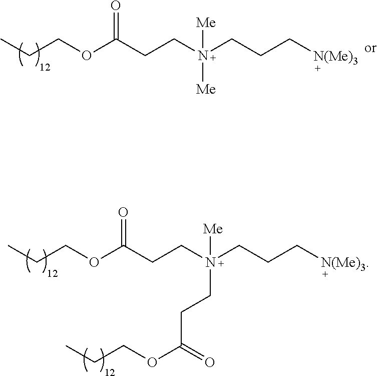

In other embodiments, the lipidoid is a composition of one or more of the above QF80 lipidoids.

In certain embodiments, the lipidoid is prepared by reacting amine 86 with an acrylamide or acrylate having a lipid tail from 9-18 carbons long. In certain embodiments, the tail is 12-15 carbons long. In certain embodiments, amine 86 is reacted with acrylate LF to form lipidoid LF86. In certain embodiments, LF86 is reacted with methyl iodide to form QF86. In certain embodiments, the lipidoid QF86 is of the formulae:

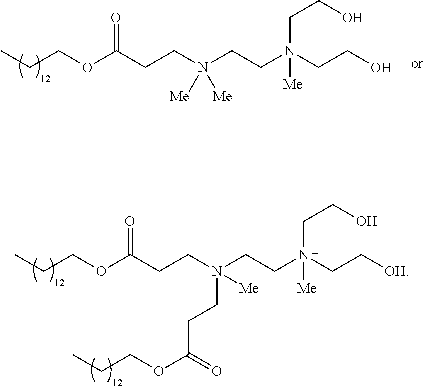

In other embodiments, the lipidoid is a composition of one or more of the above QF86 lipidoids.

In certain embodiments, the lipidoid is prepared by reacting amine 87 with an acrylamide or acrylate having a lipid tail from 9-18 carbons long. In certain embodiments, the tail is 12-15 carbons long. In certain embodiments, amine 87 is reacted with acrylate LF to form lipidoid LF87. In certain embodiments, LF87 is reacted with methyl iodide to form QF87. In certain embodiments, the lipidoid QF87 is of the formulae:

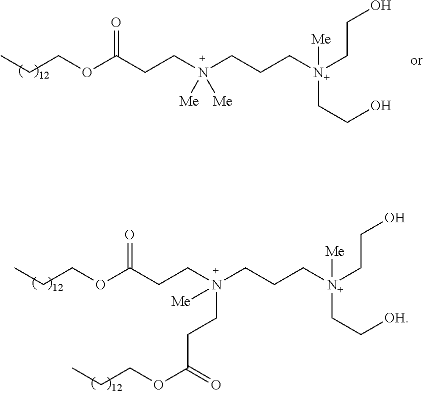

In certain embodiments, amine 87 is reacted with acrylate LG to form lipidoid LG87. In certain embodiments, LG87 is reacted with methyl iodide to form QG87. In certain embodiments, the lipidoid QG87 is of the formulae:

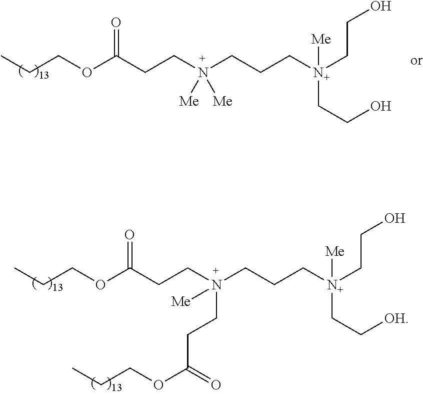

In other embodiments, the lipidoid is a composition of one or more of the above QF87 or QG87 lipidoids.

In certain embodiments, the lipidoid is prepared by reacting amine 91 with an acrylamide or acrylate having a lipid tail from 9-18 carbons long. In certain embodiments, the tail is 12-15 carbons long. In certain embodiments, amine 91 is reacted with acrylate LF to form lipidoid LF91. In certain embodiments, LF91 is reacted with methyl iodide to form QF91. In certain embodiments, the lipidoid QF91 is of the formulae:

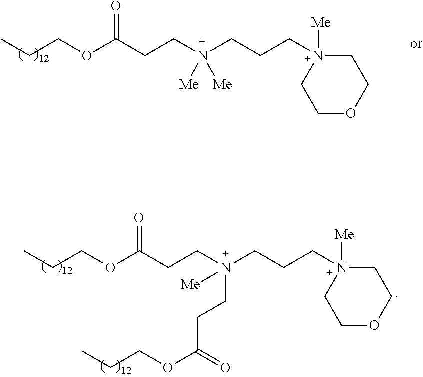

In other embodiments, the lipidoid is a composition of one or more of the above QF91 lipidoids.

In certain embodiments, the lipidoid is prepared by reacting amine 94 with an acrylamide or acrylate having a lipid tail from 9-18 carbons long. In certain embodiments, the tail is 12-15 carbons long. In certain embodiments, amine 94 is reacted with acrylate LF to form lipidoid LF94. In certain embodiments, LF94 is reacted with methyl iodide to form QF94. In certain embodiments, the lipidoid QF94 is of the formulae:

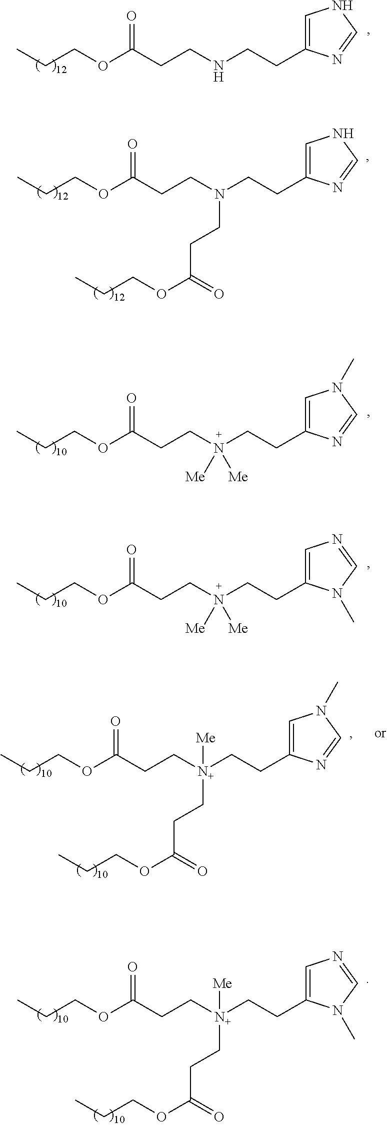

In other embodiments, the lipidoid is a composition of one or more of the above QF94 lipidoids.

### Polynucleotide Complexes

The ability of cationic compounds to interact with negatively charged polynucleotides through electrostatic interactions is well known. Cationic lipids such as Lipofectamine have been prepared and studied for their ability to complex and transfect polynucleotides. The interaction of the lipid with the polynucleotide is thought to at least partially prevent the degradation of the polynucleotide. By neutralizing the charge on the backbone of the polynucleotide, the neutral or slightly-positively-charged complex is also able to more easily pass through the hydrophobic membranes (e.g., cytoplasmic, lysosomal, endosomal, nuclear) of the cell.

The lipidoids of the present invention possess amines. Although these amines are sometimes hindered, they are available to interact with a polynucleotide (e.g., DNA, RNA, synthetic analogs of DNA and/or RNA, DNA/RNA hybrids, etc.), particularly when protonated. Polynucleotides or derivatives thereof are contacted with the lipidoids under conditions suitable to form polynucleotide/lipidoid complexes. The lipidoid is preferably at least partially protonated so as to form a complex with the negatively charged polynucleotide. In certain embodiments, the polynucleotide/lipidoid complexes form microparticles, nanoparticles, or picoparticles that are useful in the delivery of polynucleotides to cells. In certain embodiments, multiple lipidoid molecules may be associated with a polynucleotide molecule. The complex may include approximately 1-100 lipidoid molecules, approximately 1-1000 lipidoid molecules, approximately 10-1000 lipidoid molecules, or approximately 100-10,000 lipidoid molecules. In certain embodiments, the complex may form a nanoparticle. In some embodiments, the diameter of the particles ranges from approximately 10-500 nm. In some embodiments, the diameter of the particles ranges from approximately 10-1200 nm. In some embodiments, the diameter of the particles ranges from approximately 50-150 nm. The complex or particle may be associated with a targeting agent as described herein.

### Polynucleotide

The polynucleotide to be complexed, encapsulated by the inventive lipids, or included in a composition with the inventive lipds may be any nucleic acid including but not limited to RNA and DNA. In certain embodiments, the polynucleotide is DNA. In other embodiments, the polynucleotide is RNA. In other embodiments, the polynucleotide is an is RNA. In other embodiments, the polynucleotide is an is RNA. In other embodiments, the polynucleotide is an shRNA. In other embodiments, the polynucleotide is microRNA. In some embodiments, the polynucleotide is CpG DNA. The polynucleotides may be of any size or sequence, and they may be single- or double-stranded. In certain embodiments, the polynucleotide is greater than 10 base pairs long. In certain embodiments, the polynucleotide is greater than 20 base pairs long. In certain embodiments, the polynucleotide is greater than 50 base pairs long. In certain embodiments, the polynucleotide is greater than 100 base pairs long. In certain embodiments, the polynucleotide is greater than 500 base pairs long. In certain other embodiments, the polynucleotide is greater than 1000 base pairs long and may be greater than 10,000 base pairs long. The polynucleotide is preferably purified and substantially pure. In certain embodiments, the polynucleotide is greater than 50% pure. In certain embodiments, the polynucleotide is greater than 75% pure. In certain embodiments, the polynucleotide is greater than 80% pure. In certain embodiments, the polynucleotide is greater than 90% pure. In certain embodiments, the polynucleotide is greater than 95% pure. In certain embodiments, the polynucleotide is greater than 98% pure. In certain embodiments, the polynucleotide is greater than 99% pure. The polynucleotide may be provided by any means known in the art. In certain embodiments, the polynucleotide has been engineered using recombinant techniques (for a more detailed description of these techniques, please see Ausubel et al. Current Protocols in Molecular Biology (John Wiley & Sons, Inc., New York, 1999); Molecular Cloning: A Laboratory Manual, 2nd Ed., ed. by Sambrook, Fritsch, and Maniatis (Cold Spring Harbor Laboratory Press: 1989); each of which is incorporated herein by reference). The polynucleotide may also be obtained from natural sources and purified from contaminating components found normally in nature. The polynucleotide may also be chemically synthesized in a laboratory. In one embodiment, the polynucleotide is synthesized using standard solid phase chemistry.

The polynucleotide may be modified by chemical or biological means. In certain embodiments, these modifications lead to increased stability of the polynucleotide. Modifications include methylation, phosphorylation, end-capping, etc.

Derivatives of polynucleotides may also be used in the present invention. These derivatives include modifications in the bases, sugars, and/or phosphate linkages of the polynucleotide. Modified bases include, but are not limited to, those found in the following nucleoside analogs: 2-aminoadenosine, 2-thiothymidine, inosine, pyrrolo-pyrimidine, 3-methyl adenosine, 5-methylcytidine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, O(6)-methylguanine, and 2-thiocytidine. Modified sugars include, but are not limited to, 2′-fluororibose, ribose, 2′-deoxyribose, 3′-azido-2′,3′-dideoxyribose, 2′,3″-dideoxyribose, arabinose (the 2′-epimer of ribose), acyclic sugars, and hexoses. The nucleosides may be strung together by linkages other than the phosphodiester linkage found in naturally occurring DNA and RNA. Modified linkages include, but are not limited to, phosphorothioate and 5″-N-phosphoramidite linkages. Combinations of the various modifications may be used in a single polynucleotide. These modified polynucleotides may be provided by any means known in the art; however, as will be appreciated by those of skill in this art, the modified polynucleotides are preferably prepared using synthetic chemistry in vitro.

The polynucleotides to be delivered may be in any form. For example, the polynucleotide may be a circular plasmid, a linearized plasmid, a cosmid, a viral genome, a modified viral genome, an artificial chromosome, etc.

The polynucleotide may be of any sequence. In certain embodiments, the polynucleotide encodes a protein or peptide. The encoded proteins may be enzymes, structural proteins, receptors, soluble receptors, ion channels, pharmaceutically active proteins, cytokines, interleukins, antibodies, antibody fragments, antigens, coagulation factors, albumin, growth factors, hormones, insulin, etc. The polynucleotide may also comprise regulatory regions to control the expression of a gene. These regulatory regions may include, but are not limited to, promoters, enhancer elements, repressor elements, TATA box, ribosomal binding sites, stop site for transcription, etc. In other embodiments, the polynucleotide is not intended to encode a protein. For example, the polynucleotide may be used to fix an error in the genome of the cell being transfected. In certain embodiments, the polynucleotide is immunostimulatory RNA (is RNA).

The polynucleotide may also be provided as an antisense agent or RNA interference (RNAi) (Fire et al. Nature 391:806-811, 1998; incorporated herein by reference). Antisense therapy is meant to include, e.g., administration or in situ provision of single- or double-stranded oligonucleotides or their derivatives which specifically hybridize, e.g., bind, under cellular conditions, with cellular mRNA and/or genomic DNA, or mutants thereof, so as to inhibit expression of the encoded protein, e.g., by inhibiting transcription and/or translation (Crooke “Molecular mechanisms of action of antisense drugs” Biochim. Biophys. Acta 1489(1):31-44, 1999; Crooke “Evaluating the mechanism of action of antiproliferative antisense drugs” Antisense Nucleic Acid Drug Dev. 10(2):123-126, discussion 127, 2000; Methods in Enzymology volumes 313-314, 1999; each of which is incorporated herein by reference). The binding may be by conventional base pair complementarity, or, for example, in the case of binding to DNA duplexes, through specific interactions in the major groove of the double helix (i.e., triple helix formation) (Chan et al. I Mol. Med. 75(4):267-282, 1997; incorporated herein by reference).

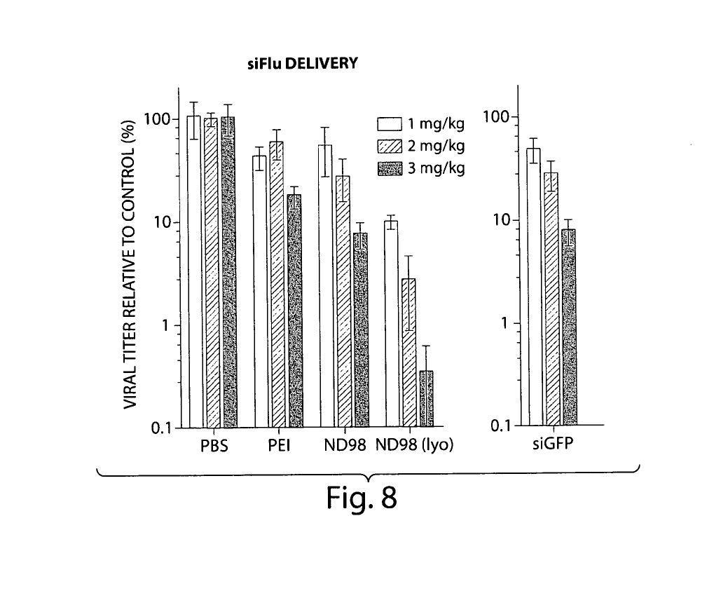

In certain embodiments, the polynucleotide to be delivered is R1362 RNA. R1362 is a single-stranded RNA with known is RNA activity. R1362 has been shown to be a strong ligand for both TLR7 and TLR8 in vitro in human PBMCs tranfected with DOTAP (Forsbach et al., I Immunol. (2008) 180: 3729-3738, incorporated herein by reference) and in vivo in mice (FIG. 8).

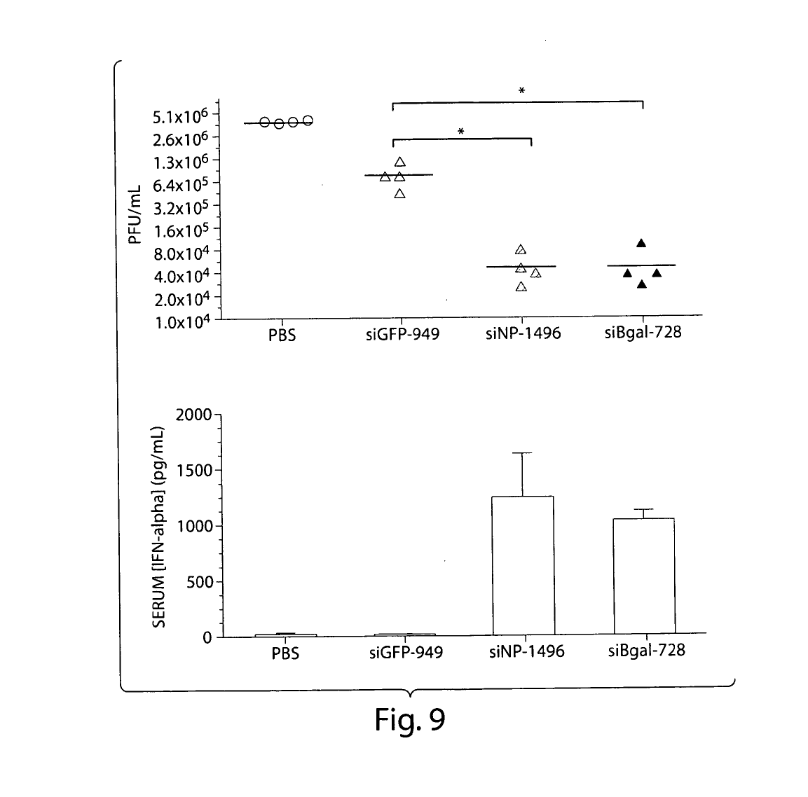

In certain embodiments, the polynucleotide to be delivered is siNP-1496. siNP-1496 has been suggested to activate human PBMCs in vitro (Judge et al. Mol. Ther. (2006) 13: 494-505, incorporated herein by reference) and in vivo in mice (FIG. 9).

In certain embodiments, the polynucleotide to be delivered is R-006. The sequence of R-006 is 5′-UUGUUGUUGUUGUUGUUGUU-3′

In some embodiments, the polynucleotide to be delivered is any one of the polynucleotides featured in Forsbach et al., J. Immunol. (2008) 180: 3729-3738, incorporated herein by reference.

In other embodiments, the polynucleotide to be delivered is any RNA molecule that interacts with one of a variety of innate immune receptors (such as the TLRs or RIG-I or other, perhaps unknown receptors) and triggers innate immune responses (e.g., release of cytokines such as IL-6, IP-10, TNF-a or interferons like interferon-alpha, interferon-beta, or interferon-gamma).

### Particles

The lipidoids of the present invention may also be used to form drug delivery devices. The lipidoids may be used to encapsulate agents including polynucleotides. The inventive lipidoids have several properties that make them particularly suitable in the preparation of drug delivery devices. These include 1) the ability of the lipid to complex and “protect” labile polynucletides; 2) the ability to buffer the pH in the endosome; 3) the ability to act as a “proton sponge” and cause endosomolysis; and 4) the ability to neutralize the charge on negatively charged polynucleotides. In certain embodiment, the lipidoids are used to form nanoparticles containing the polynucleotides to be delivered. These nanoparticles may include other materials such as proteins, carbohydrates, synthetic polymers (e.g., PEG, PLGA), lipids, and natural polymers. In certain embodiments, the diameter of the particle ranges from between 500 nm to 50 micrometers. In certain embodiments, the diameter of the particle ranges from 1 micrometer to 20 micrometers. In certain embodiments, the diameter of the particle ranges from 1 micrometer to 10 micrometers. In certain embodiments, the diameter of the particle ranges from 1-5 micrometers. In certain embodiments, the diameter of the particle ranges from between 10 nm to 500 nm. In certain embodiments, the diameter of the particle ranges from between 100 nm to 1200 nm. In certain embodiments, the diameter of the particle ranges from between 50 nm to 150 nm.

### Methods of Preparing Particles

The particles of the invention may be prepared using any method known in this art. These include, but are not limited to, spray drying, single and double emulsion solvent evaporation, solvent extraction, phase separation, simple and complex coacervation, and other methods well known to those of ordinary skill in the art. In some embodiments, the particles are prepared by the double emulsion process or spray drying. The conditions used in preparing the particles may be altered to yield particles of a desired size or property (e.g., hydrophobicity, hydrophilicity, external morphology, “stickiness”, shape, etc.). The method of preparing the particle and the conditions (e.g., solvent, temperature, concentration, air flow rate, etc.) used may also depend on the agent being encapsulated and/or the composition of the matrix.

Methods developed for making particles for delivery of encapsulated agents are described in the literature (for example, please see Doubrow, M., Ed., “Microcapsules and Nanoparticles in Medicine and Pharmacy,” CRC Press, Boca Raton, 1992; Mathiowitz and Langer, J. Controlled Release 5:13-22, 1987; Mathiowitz et al. Reactive Polymers 6:275-283, 1987; Mathiowitz et al. J. Appl. Polymer Sci. 35:755-774, 1988; each of which is incorporated herein by reference).

If the particles prepared by any of the above methods have a size range outside of the desired range, the particles can be sized, for example, using a sieve. The particle may also be coated. In certain embodiments, the particles are coated with a targeting agent. In other embodiments, the particles are coated to achieve desirable surface properties (e.g., a particular charge).

### Targeting Agents

The particles of the invention may be modified to include targeting agents since it is often desirable to target a particular cell, collection of cells, or tissue. A variety of targeting agents that direct pharmaceutical compositions to particular cells are known in the art (see, for example, Cotten et al. Methods Enzym. 217:618, 1993; incorporated herein by reference). The targeting agents may be included throughout the particle or may be only on the surface. The targeting agent may be a protein, peptide, carbohydrate, glycoprotein, lipid, small molecule, etc. The targeting agent may be used to target specific cells or tissues or may be used to promote endocytosis or phagocytosis of the particle. Examples of targeting agents include, but are not limited to, antibodies, fragments of antibodies, low-density lipoproteins (LDLs), transferrin, asialycoproteins, gp120 envelope protein of the human immunodeficiency virus (HIV), carbohydrates, receptor ligands, sialic acid, aptamers, etc. If the targeting agent is included throughout the particle, the targeting agent may be included in the mixture that is used to form the particles. If the targeting agent is only on the surface, the targeting agent may be associated with (i.e., by covalent, hydrophobic, hydrogen bonding, van der Waals, or other interactions) the formed particles using standard chemical techniques.

### Uses of Lipidoid Complexes and Pharmaceutical Compositions Thereof

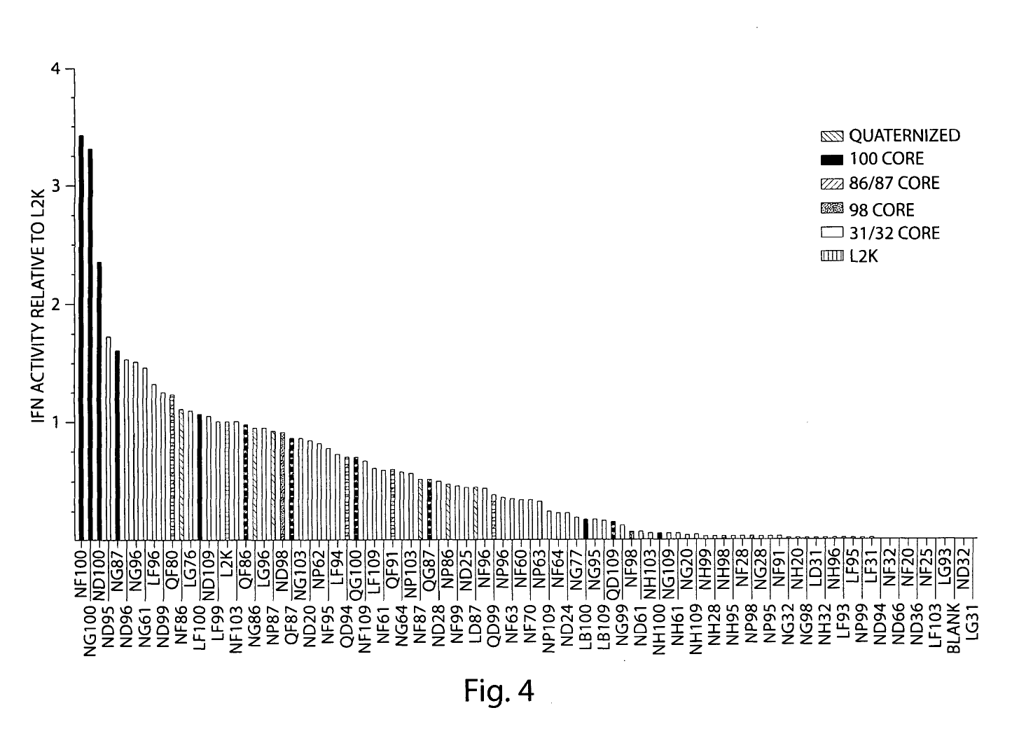

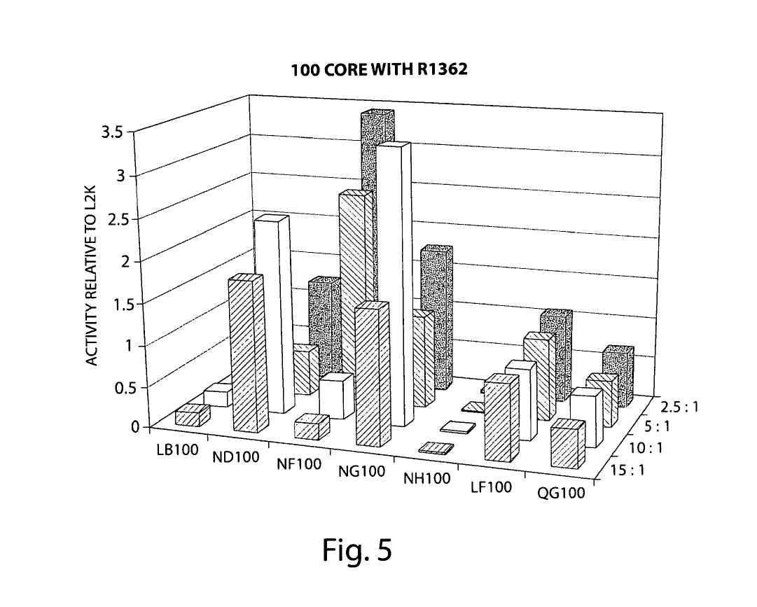

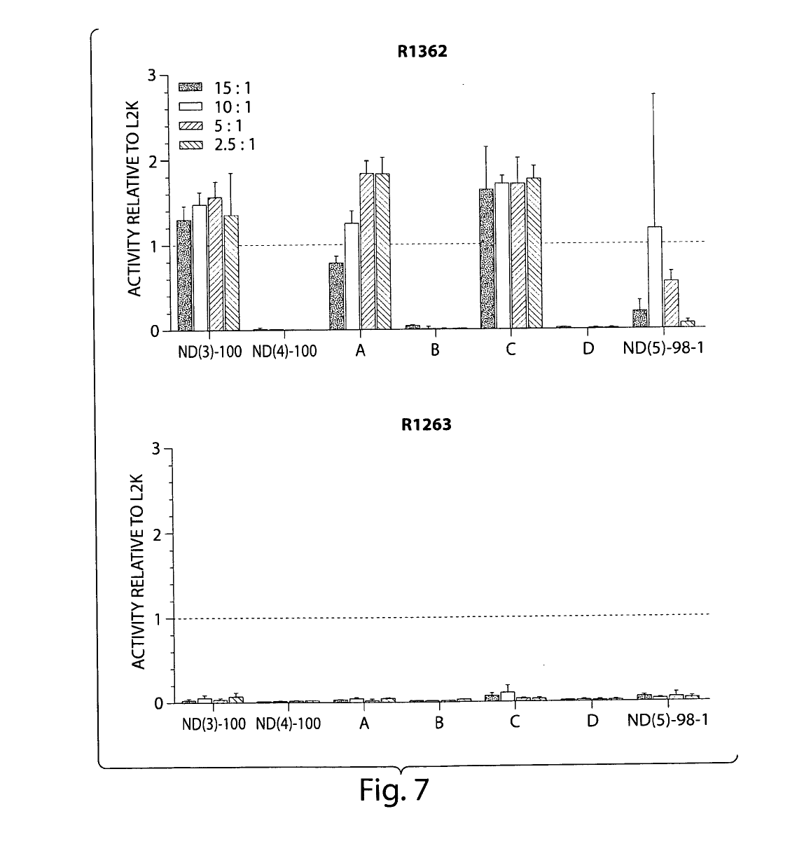

The lipidoids of the current invention have the ability to functionally deliver single-stranded and double-stranded RNA as well as CpG DNA oligonucleotides to a cell. For example, lipidoids based on amine 100 (see FIG. 1 for amines) are efficient at delivering RNA in an immunostimulatory manner (FIGS. 4, 5, and 7), and some are more efficient at is RNA delivery than Lipofectamine 2000 (L2K), which is a commercially available transfection reagent (FIG. 4). Human peripheral blood mononuclear cells (PBMC) treated with amine 100 lipidoid-RNA nanoparticles efficiently generated high amounts of interferon-alpha (FIG. 5), which is characteristic of activating TLR7/9 in the plasmacytoid dendritic cell, part of the innate immune response. Changing the tails attached to amine 100 modified the immunostimulatory characteristics (FIG. 7). ND100 is a very strong activator, as are amine 100 materials with mixed ND and other tails (NC, NF, NG, etc.). Some lipidoid materials may also directly activate the innate immune system independent of nucleic acid delivery, such as materials based on the amine 86 and amine 87 cores.

The immune responses generated by lipidoid-RNA nanoparticles have antiviral properties and increase adaptive immune responses to vaccination (i.e., function as a vaccine adjuvant). For example, materials based on ND98 have been used for RNA interference previously (Akinc et al., Nat Biotechnol (2008) 26: 561-569). Nanoparticle formulations of ND98 impart a novel function of immune stimulation (FIGS. 8 and 9). Lipidoids of the current invention may also be useful to activate immune-mediated anti-tumor activity.

The inventive compositions may be used in the treatment of cancer. Examples of cancers treated with compositions of the present invention include solid and hematological tumors. Solid tumors are exemplified by tumors of the breast, bladder, bone, brain, central and peripheral nervous system, colon, connective tissue, endocrine glands (e.g., thyroid and adrenal cortex), esophagus, endometrium, germ cells, head and neck, kidney, liver, lung, larynx and hypopharynx, mesothelioma, muscle, ovary, pancreas, prostate, rectum, renal, small intestine, soft tissue, testis, stomach, skin, ureter, vagina, and vulva. Inherited cancers exemplified by retinoblastoma and Wilms tumor are also included. In addition, cancers include primary tumors in said organs and corresponding secondary tumors in distant organs (“tumor metastases”). Hematological tumors are exemplified by aggressive and indolent forms of leukemia and lymphoma, namely non-Hodgkins disease, chronic and acute myeloid leukemia (CML/AML), acute lymphoblastic leukemia (ALL), chronic lymphocytic leukemia (CLL), Hodgkins disease, multiple myeloma, and T-cell lymphoma. Also included are myelodysplastic syndrome, plasma cell neoplasia, paraneoplastic syndromes, cancers of unknown primary site as well as AIDS-related malignancies.

As these materials efficiently deliver immunostimulatory polynucleotides (e.g., is RNA) to activate immune responses, they could also be used to deliver antagonists of immune responses that have been described by others (Robbins et al., Mol Ther (2007) 15: 1663-1669, incorporated herein by reference). Delivery of immune inhibitory nucleic acids using lipidoids could modify or suppress immune activity, which would be useful in diseases of autoimmune diseases or inflammatory diseases (Krieg et al., Immunity (2007) 27: 695-697; incorporated herein by reference).

In certain embodiments, the present invention provides methods for treating or lessening the severity of autoimmune diseases including, but not limited to, inflammatory bowel disease, arthritis, systemic lupus erythematosus, rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, Sjogren's syndrome, multiple sclerosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, celiac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, endometriosis, interstitial cystitis, neuromyotonia, scleroderma, or vulvodynia.

In some embodiments, the present invention provides a method for treating or lessening the severity of an inflammatory disease including, but not limited to, asthma, appendicitis, Behcet's disease, Blau syndrome, blepharitis, bronchiolitis, bronchitis, bursitis, cervicitis, cholangitis, cholecystitis, chronic recurrent multifocal osteomyelitis (CRMO), colitis, conjunctivitis, cryopyrin associated periodic syndrome (CAPS), cystitis, dacryoadenitis, dermatitis, dermatomyositis, encephalitis, endocarditis, endometritis, enteritis, enterocolitis, epicondylitis, epididymitis, familial cold-induced autoinflammatory syndrome, familial Mediterranean fever (FMF), fasciitis, fibrositis, gastritis, gastroenteritis, hepatitis, hidradenitis suppurativa, laryngitis, mastitis, meningitis, mevalonate kinase deficiency (MKD), Muckle-Well syndrome, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, pyoderma gangrenosum and acne syndrome (PAPA), pyogenic sterile arthritis, rhinitis, salpingitis, sinusitis, stomatitis, synovitis, systemic juvenile rheumatoid arthritis, tendonitis, TNF receptor associated periodic syndrome (TRAPS), tonsillitis, uveitis, vaginitis, vasculitis, or vulvitis.

Once the complex or particles have been prepared, they may be combined with one or more pharmaceutical excipients to form a pharmaceutical composition that is suitable to administer to animals including humans. As would be appreciated by one of skill in this art, the excipients may be chosen based on the route of administration as described below, the agent being delivered, time course of delivery of the agent, etc.

Pharmaceutical compositions of the present invention and for use in accordance with the present invention may include a pharmaceutically acceptable excipient or carrier. As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions of this invention can be administered to humans and/or to animals, orally, rectally, parenterally, intracisternally, intravaginally, intranasally, intraperitoneally, topically (as by powders, creams, ointments, or drops), bucally, or as an oral or nasal spray.

Liquid dosage forms for oral administration include pharmaceutically acceptable emulsions, microemulsions, solutions, suspensions, syrups, and elixirs. In addition to the active ingredients (i.e., microparticles, nanoparticles, liposomes, micelles, polynucleotide/lipid complexes), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. In a particularly preferred embodiment, the particles are suspended in a carrier fluid comprising 1% (w/v) sodium carboxymethyl cellulose and 0.1% (v/v) Tween 80.

The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration are preferably suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the microparticles.

Solid dosage forms for oral administration include capsules, tablets, pills, powders, and granules. In such solid dosage forms, the particles are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Solid compositions of a similar type may also be employed as fillers in soft and hard-filled gelatin capsules using such excipients as lactose or milk sugar as well as high molecular weight polyethylene glycols and the like.

Dosage forms for topical or transdermal administration of an inventive pharmaceutical composition include ointments, pastes, creams, lotions, gels, powders, solutions, sprays, inhalants, or patches. The particles are admixed under sterile conditions with a pharmaceutically acceptable carrier and any needed preservatives or buffers as may be required. Ophthalmic formulation, ear drops, and eye drops are also contemplated as being within the scope of this invention.

The ointments, pastes, creams, and gels may contain, in addition to the particles of this invention, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the particles of this invention, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the microparticles or nanoparticles in a proper medium. Absorption enhancers can also be used to increase the flux of the compound across the skin. The rate can be controlled by either providing a rate controlling membrane or by dispersing the particles in a polymer matrix or gel.

These and other aspects of the present invention will be further appreciated upon consideration of the following Examples, which are intended to illustrate certain particular embodiments of the invention but are not intended to limit its scope, as defined by the claims.

### EXAMPLES

### Example 1

### Combinatorial Lipidoid Synthesis

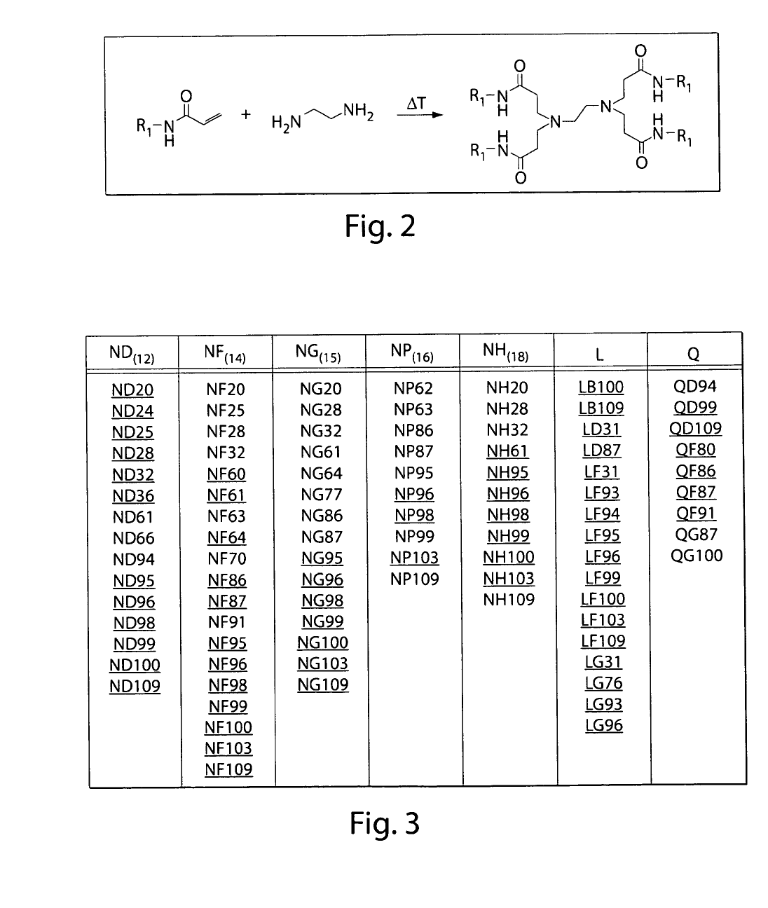

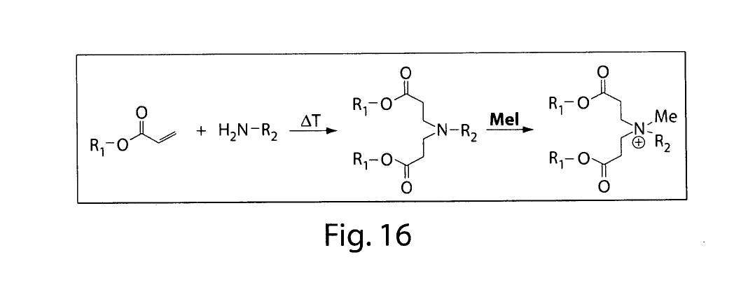

Lipidoids were synthesized as depicted in FIG. 2 under solvent-free conditions by reacting primary and secondary amine-containing cores (FIG. 1a, right) with alkyl-acrylate or alkyl-acrylamide (FIG. 1a, left) tails at a high tail-to-core monomer ratio to drive synthesis of fully- and (n−1)-substituted lipidoids. Lipidoid products were purified of unreacted core and side-chain reactants resulting in crude mixtures of undefined relative compositions of fully and incompletely-substituted lipidoids. Some alkyl-acrylate-tail lipidoids were further reacted with methyl iodide (FIG. 16) to form quaternized amines with a permanent positive charge. Promising lipidoids for further study were purified. Nomenclature reflects alkyl tail linkage (ester=L, amide=N), alkyl tail carbon length (A=9, B=10, D=12, F=14, G=15, P=16, H=18 carbons), and amine-containing core. Quaternized core amines are further referred to with a Q designation instead of L. Purified lipidoids include number of tails in parenthesis ( ) following tail name. A complete list of crude lipidoids screened is found in FIG. 3.

Second generation lipidoids were synthesized in a four-step process (FIG. 6). The primary amine was protected on one side by reacting 10× molar excess pure amine 100 with di-tert-butyl dicarbonate (Boc₂O). ND tails were reacted with the free primary amine in excess prior to deprotection and regeneration of the opposite primary amine resulting in ND(2)-100. ND(2)-100 was further reacted with NA or LD tails and purified into 3-tail or 4-tail derivatives, which have been renamed lipidoids A-D for clarification.

### High-Throughput Screening for Lipidoid-Mediated is RNA Delivery

Donor-blind buffy-coat packs were obtained from the Massachusetts General Hospital blood bank. Peripheral blood mononuclear cells (PBMC) were obtained by Ficoll-Paque Plus (Amersham Biosciences) density centrifugation at 400 RCF and washed twice in PBS with 2 mM EDTA. PBMCs were resuspended in supplemented RPMI (RPMI 1640 medium with 10% FCS, 1 mM MEM sodium pyruvate, 10 mM HEPES, and 100 U/mL penicillin/streptomycin) and plated at 5×10⁵ cells/well in 175 uL in 96-well tissue culture plates. Lipofectamine 2000 (L2K) (Invitrogen) was used as a positive control for transfection of RNA according to manufacturers protocols and to normalize interferon responses across different donors.

Crude or purified lipidoid products and RNA were dissolved in 25 mM sodium acetate, pH 5, to 0.5 mg/mL. For lipidoids with poor solubility, up to 10% DMSO was added to stock lipidoid solutions; sonication was also used to increase solubility. Lipidoid solution was arrayed in a 96-well round bottom reaction plate and mixed at 15, 10, 5, and 2.5:1 mass ratios of lipid to RNA (at 50 μg/mL also in sodium acetate) for 80 μL total volume. After 20 minutes incubation at room temperature to allow for nanoparticle complexes to form, 120 μL RPMI media was added to dilute complexes and buffer sodium acetate. Diluted complexes in the amount of 25 μL were then added to PBMCs for a final RNA concentration of 200 ng RNA per well in 200 μL, media (1 μg/mL˜140 nM). Following 16-20 hours of incubation, supernatants were taken from PBMC cultures after centrifuging at 400 RCF for 10 minutes and stored in 96-well plates at −80° C. for later quantification. Transfections were performed in quadruplicate for each weight ratio and both immunostimulatory R1362 and control R1263 RNA.

To quantify Type 1 interferon activity, a HTS-compatible cell-based detection assay was utilized. Briefly, 293T-ISRE-RFP cells were incubated with 50 μL PBMC supernatant overnight prior to quantification of red fluorescence signal. Recombinant human interferon alpha serially diluted in supplemented RPMI was used as a standard, and type I interferon activity of each screening well was normalized to activity from L2K transfections. Results are shown in FIGS. 4, 5, and 7.

### Lipidoid-RNA Nanoparticle Formulation and Characterization

Purified lipidoid was dissolved to 120 mg/mL in ethanol, cholesterol (Ch) (Sigma Aldrich, St. Louis, Mo.) was dissolved to 25 mg/mL in ethanol, and N-palmitoyl-sphingosine-1-[succinyl(methoxypolyethylene glycol)2000] (C16 mPEG 2000 ceramide) (“PEG”) (Avanti Polar Lipids, Alabaster, Ala.) was dissolved to 100 mg/mL in ethanol. Lipidoid, Ch, and PEG were combined at a 15:0.8:7 mass ratio (L:C:P), vortexed briefly, and diluted in a mixture of ethanol and 200 mM sodium acetate (with 16.67 mg/mL sucrose for lyophilization) for a final lipidoid concentration of 7.5 mg/mL in 35% ethanol, 65% NaAc. RNAs was resuspended in H2O to 10 mg/mL and diluted to 35% ethanol. Lipidoid/Ch/PEG were added to diluted RNA at a 15, 11.5, or 10:1 mass ratio (L:R) and vortexed for 20 minutes to allow complexes to form. Complexed lipidoid-RNA nanoparticles were then extruded once through a double 200 nm membrane and then twice through a double 80 nm membrane (Whatman, Florham Park, N.J.) on a Northern Lipids (British Columbia, Canada) extrusion system at 40° C. To remove ethanol prior to injection, nanoparticles were dialyzed in a Slide-A-Lyzer 3500 MWCO dialysis cassette (Pierce Biotech) against PBS. For lyophilization, 10 mg of sucrose was added per mL of extruded complexes prior to freezing at −80° C. for >2 hours followed by >1 day lyophilization.

For quanitification and encapsulation efficiency of RNA, a 50 μL sample of nanoparticles was diluted 200-fold in Tris-EDTA buffer (TE), mixed with either 50 μL of TE buffer or 504 of 2% Triton-X-100 (T-X) in TE, and incubated with 100 uL Quant-It Ribogreen reagent (Invitrogen) according to the manufacturers protocols for 20 minutes at 37° C. in a 96-well plate. Fluoroescence intensity was determined at 485 nm (ex)/535 nm 9 em). Total fluorescence in the presence of T-X was compared to a standard curve of RNA diluted in TE to determine total RNA concentration; RNA encapsulation efficiency was determined by the ratio of fluorescence signal without T-X and with T-X. Binding affinity of formulation lipidoid complexes was investigated by measuring Ribogreen signal following incubation of nanoparticles with a dilution series of T-X between 0 and 4% T-X.

Nanoparticle size and zeta-potential of formulated lipidoid-RNA nanoparticles was assayed by light scattering using a Zeta-PALS instrument (Brookhaven Instruments) after 1/50 dilution in PBS or with a Mastersizer instrument (Malvern Instruments) after dilution in HBSS.

### In Vivo Characterization of Innate Immune Responses

All studies were conducted at Coley Pharmaceuticals (Ottowa, ON, Canada) under the approval of the institutional care committees and in accordance with the guidelines set forth by the Canadian Council on Animal Care. Formulated lipidoid-RNA nanoparticles were resuspended or diluted in HBSS prior to injection under isofluorane anesthesia. Innate immune responses of multiple lipidoid formulations with R1362 RNA were compared after subcutaneous (SC) injections in BALB/c mice. To further investigate RNA-specific immune responses, nanoparticles were injected SC at increasing doses or intravenously (IV) into 129sv mice. TLR-specific responses were investigated following IV injection in C57Bl/6, C57Bl/6 TLR7^{-/-}, C57Bl/6 TLR9^{-/-}, C57Bl/6 MyD88^{-/-}, C3H, or C3H TLR4^{-/-}. As a control, DOTAP (1,2-dioleoyloxy-3-(trimethylammonium)propane) (Roche) was complexed with RNA at a 2:1 weight ratio (L:R).

Blood samples for serum isolation were taken by direct cardiac puncture on heparin at indicated timepoints. Plasma samples were analyzed by ELISA with commercially available antibodies (IFN-alpha, PBL Labs) (IP-10, BD Pharmingen) or 10-plex Luminex technology (BioSource International). Spleenocytes were also harvested for surface expression of activation markers by staining with anti-CD69-FITC, anti-CD86-APC, anti-CD3-PE/Cy7, anti-CD19-ECD, and anti-DX5-PE.

### Lipidoid-RNA Nanoparticles Adjuvant Adaptive Immune Responses

Lipidoid-RNA nanoparticles were formulated in liquid form with either R1362 or R1263 at a 15:1 ratio (lipidoid to RNA) and dialyzed to remove ethanol. Lipidoid-RNA nanoparticles with 28 ug of RNA were mixed with 20 ug chicken ovalbumin protein antigen (Ova) immediately prior to IM injection into C57Bl/6 mice (n=10). Control mice were vaccinated with Ova protein antigen alone, and vaccination with the TLR9 agonist CpG 1826 ODN mixed with Ova antigen was investigated for comparison. Mice were dosed three times, at days 0, 14, and 21. After 28 days, blood was collected by direct cardiac puncture on heparin (n=10). Plasma antibody levels of anti-Ova IgG1 and IgG2c were determined by sandwich ELISA.

Spleenocytes (SC) were harvested for flow cytometric analysis of surface expression markers and cytokine production. SC (n=5) were directly stained for antigen-specific T-cells with CD44-FITC, SIINFEKL-tetramer-PE, CD8-ECD, CD62L-APC, PD-1-biotin+strepavidin-PE/Cy7. The same SC samples were independently stimulated in vitro in RPMI-1640+1% L-glutamine+1% Pen/Strep+10% FBS+β-mercaptoethanol with 5 μg/mL SIINFEKL peptide for 1 hour, stimulated with Brefeldin A (1 μg/mL) for 4 hours, and then stained with SIINFEKL tetramer-PE and CD8-ECD followed by intracellular staining with IL2-FITC, TNF-APC, and IFNγ-PECy7.

### Dose-Dependent Inhibition of Influenza Viral Replication in Mouse Lung

Mice were injected IV twice with freshly prepared unlyophilized ND(5)-98-1 (ND98), PEI nanoparticles, or PBS at 1, 2, or 3 mg/kg siRNA prior to challenge with 12,000 PFU of influenza virus. ND(5)-98-1 is a lipidoid based on amine 98 that contains five ND tails. Lung viral titer was determined by quantitative plaque forming assay. Results are shown in FIG. 8. Up to 300-fold inhibition of viral replication was observed with lyophilized ND98 nanoparticle formulations delivering siRNA in an immunostimulatory fashion. As a control, ND98 nanoparticles made with siRNA against GFP, which is less immunostimulatory, exhibited less antiviral activity.

Inhibitory Effect of Lipidoid Nanoparticles Correlates with Induction of Systemic Type 1 Interferon Response

Mice were injected IV twice with lyophilized ND(5)-98-1 (ND98)-siRNA nanoparticles at 2 mg/kg siRNA prior to (a) infection with 12,000 PFU, or (b) blood collection by cardiac puncture. Results are shown in FIG. 9. (a) Lung viral titer was determined by quantitative plaque forming assay. (b) Serum IFN-alpha concentration was determined by ELISA. Nanoparticles with siNP-1496 (immunostimulatory) or unmatched siBgal-728 sequence (immunostimulatory) elicited high levels of IFN-alpha that corresponded with significant reductions in viral titer over nanoparticles formulated with siGFP-949 (control non-stimulatory).

Sequence of siNP-1496:
5′-GGAUCUUAUUUCUUCGGAGUU-3′.
Sequence of siBgal-728:
5′-CUACACAAAUCAGCGAUUUUU-3′.
Sequence of siGFP-949:
5′-GGCUACGUCCAGGAGCGCAUU-3′.

### Example 2

### Development of Lipidoids for Immunostimulatory RNA Drug Delivery Introduction

Innate immune activation is a crucial step in activating mammalian responses to microbial infection ultimately leading to protective adaptive immunity. Activation of pattern recognition receptors (PRRs) allows for rapid identification of common pathogen-associated molecular patterns (PAMPs) without the need for prior education of an adaptive response.^{1,2} The Toll-like receptors (TLR), of which eleven have been identified in humans, recognize conserved structures among a diverse group of pathogens such as long dsRNA (TLR3), lipopolysachharide of bacterial cell walls (TLR4), and flagella (TLR5).² Nucleic acids can be recognized by TLRs 7, 8, and 9, which comprise a closely related genetic sub-family whose expression is species-dependent, cell-type specific, and is functionally compartmentalized to the endosome.³ TLR9 recognizes CpG sequences in unmethylated bacterial or viral DNA and synthetic CpG oligodeoxynucleotides (ODN). The activation of TLRs 7 and 8 is a hallmark of innate immune activation by RNA viruses. In humans, TLR7 seems to be highly expressed and functional mainly in plasmacytoid dendritic cells (pDCs) and B-cells, while TLR8 expression is localized mostly to monocytes, myeloid DCs (mDCs), and monocyte-derived DCs (moDCs).⁴

In particular, recognition of bacterial and viral nucleic acids in the endosomal compartment of plasmacytoid dendritic cells (TLR7 and TLR9) and myeloid dendritic cells (TLR3 and TLR8) results in a characteristic type I interferon response and coordinated Th1-biasing cytokine profile.¹ This innate immune response can have clinically relevant effects by activating anti-viral defenses² and increasing immune surveillance of cancer.⁵ Vaccine adjuvants can function by increasing coupling of innate and adaptive responses or by directing sustained availability of antigen to specific cells in the lymph nodes.⁷ Activation of the innate immune system is a critical step in generation of the second signal, or “danger signal,” necessary for efficient production of specific T-cell mediated responses and class switching to high-affinity antibodies.^{1,8} Thus, TLR activation may be useful for adjuvanting immune responses to vaccines.

Small-molecule agonists of TLR7 and TLR8 such as imiquimod and R-848 have been in used in the clinic as cancer therapies.^{5,9} However, therapeutic activation of TLR7 and TLR8 with small single-stranded RNAs, the natural ligands for TLR7/8,^{10, 11} has proven difficult due to low stability, nuclease degradation,¹²⁻¹⁴ and the requirement of endosomal uptake^{10, 15}. Many groups have focused on strategies to chemically or physically alter ssRNA and siRNA to increase serum stability and increase circulation time, but these modifications may also inhibit TLR activation^{14, 16-18} or require impractically large dosing¹² to achieve immunostimulatory effects.

We hypothesized that controlled delivery of ssRNA to TLR7 or TLR8 could mimic the robust adaptive immune responses triggered by viral infection through efficient activation of innate immune responses.

Controlling the cellular uptake and intracellular location of ssRNA are crucial steps to induction of TLR7/8 responses.^{15,20} Typically, DOTAP (N-[1-(2,3-dioleoyloxy)propyl]-N,N,Ntrimethylammonium methylsulfate) has been used to facilitate cellular uptake and to investigate immunostimulatory RNA (is RNA) effects in vitro.^{10,11,14,21,23} Many non-viral methods for gene delivery in vivo have also been developed utilizing various materials such as polymers²⁴ and lipids,²⁵ but no systems designed specifically for delivery of is RNAs to TLRs currently exist. As the design criteria for is RNA delivery to TLR7/8 in the endosome are different from that of DNA delivery and siRNA delivery to the cytosol, we approached this problem by high throughput screening of a promising new class of cationic lipid-like materials for RNA delivery,²⁶ termed “lipidoids” (see U.S. patent application U.S. Ser. No. 11/453,222, filed Jun. 14, 2006, which is incorporated herein by reference).

These lipidoids are based upon a combinatorial library of amine-containing hydrophilic cores and hydrophobic tails. We performed in vitro screening for is RNA-mediated innate activation in primary human cells and quantified type I interferon production using a cell-based reporter assay.²⁷ A novel lipidoid structural motif was discovered that enhanced is RNA-mediated stimulation of type I interferon secretion. Second generation lipidoids were then synthesized based upon this motif and then optimized for innate immune activation in vivo. Optimized lipidoid-RNA nanoparticle formulations were highly efficient for delivery of is RNA in a variety of mouse strains. Subcutaneous injections lead to RNA-specific stimulation of type I IFN secretions, induction of cytokine profiles characteristic of delivery to specific dendritic cell classes, and activation of splenic lymphocytes. Innate immune activation was sufficient to induce an antiviral state and provide resistance to experimental influenza infection. We further investigated the nature of the immune response to lipidoid-mediated is RNA delivery in knockout mouse models of various TLR receptors and MyD88, a common signaling intermediate shared by most TLRs.²⁰ Finally, we applied lipidoid-mediated delivery of is RNAs to adjuvant responses to intramuscular protein administration achieving highly efficient induction of both cellular and humoral immune responses.

### Results

### High-Throughput Screening Reveals Lipidoids Capable of In Vitro is RNA Delivery

The therapeutic use of nucleic acids requires a mechanism for protection against physiologic nucleases and delivery across cellular and tissue barriers. A variety of lipidoids molecules were generated by combinatorial addition of alkyl-acrylate or alkyl-acrylamide tails to primary or secondary amine-containing cores (FIG. 1) following a simple solvent-free synthesis (FIG. 2). A preliminary library consisting of 96 lipidoid combinations (Table 1) included a variety of carbon tail lengths and amine-core structures.

TABLE 1
List of the 96 crude lipidoids screened.

| ND₍₁₂₎ | NF₍₁₄₎ | NG₍₁₅₎ | NP₍₁₆₎ | NH₍₁₈₎ | L | Q |
|---|---|---|---|---|---|---|
| ND20 | NF20 | NG20 | NP62 | NH20 | LB100 | QD94 |
| ND24 | NF25 | NG28 | NP63 | NH28 | LB109 | QD99 |
| ND25 | NF28 | NG32 | NP86 | NH32 | LD31 | QD109 |
| ND28 | NF32 | NG61 | NP87 | NH61 | LD87 | QF80 |
| ND32 | NF60 | NG64 | NP95 | NH95 | LF31 | QF86 |
| ND36 | NF61 | NG77 | NP96 | NH96 | LF93 | QF87 |
| ND61 | NF63 | NG86 | NP98 | NH98 | LF94 | QF91 |
| ND66 | NF64 | NG87 | NP99 | NH99 | LF95 | QG87 |
| ND94 | NF70 | NG95 | NP103 | NH100 | LF96 | QG100 |
| ND95 | NF86 | NG96 | NP109 | NH103 | LF99 | |
| ND96 | NF87 | NG98 | | NH109 | LF100 | |
| ND98 | NF91 | NG99 | | | LF103 | |
| ND99 | NF95 | NG100 | | | LF109 | |
| ND100 | NF96 | NG103 | | | LG31 | |
| ND109 | NF98 | NG109 | | | LG76 | |
| | NF99 | | | | LG96 | |
| | NF100 | | | | LG93 | |
| | NF103 | | | | | |
| | NF109 | | | | | |

The lipidoids were complexed with short single-stranded is RNA²¹ at 4 mass ratios of lipid to RNA and screened in vitro for delivery in human PBMCs. RNA R1362 is a GU-rich sequence that is a highly active TLR7 & TLR8 agonist resulting in production of type I interferon and Th1-type cytokines.²¹ To control for direct lipid-mediated immunostimulatory activity or toxicity, we also screened with the R1263 ssRNA sequence that exhibits low TLR7 and TLR8 activity. Secretion of Type I IFN was quantified using a high-throughput cell-based assay,²⁷ and lipidoid-mediated activity was normalized to Lipofectamine 2000 (L2K)-mediated transfection with R1362 to control for donor PBMC variability in type I interferon secretion capacity.

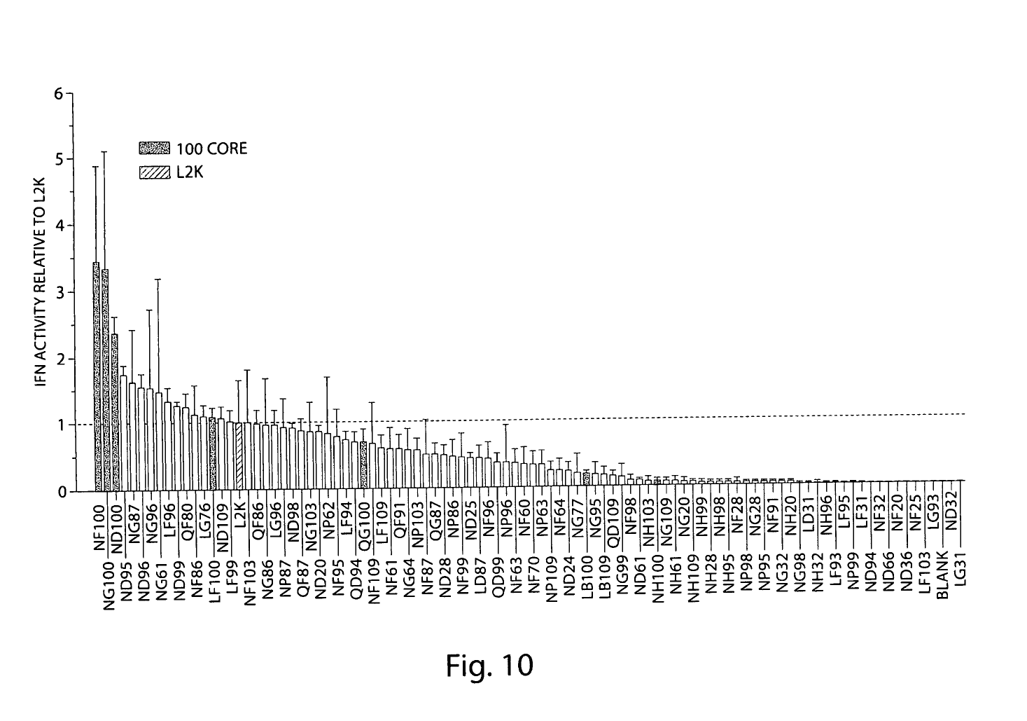

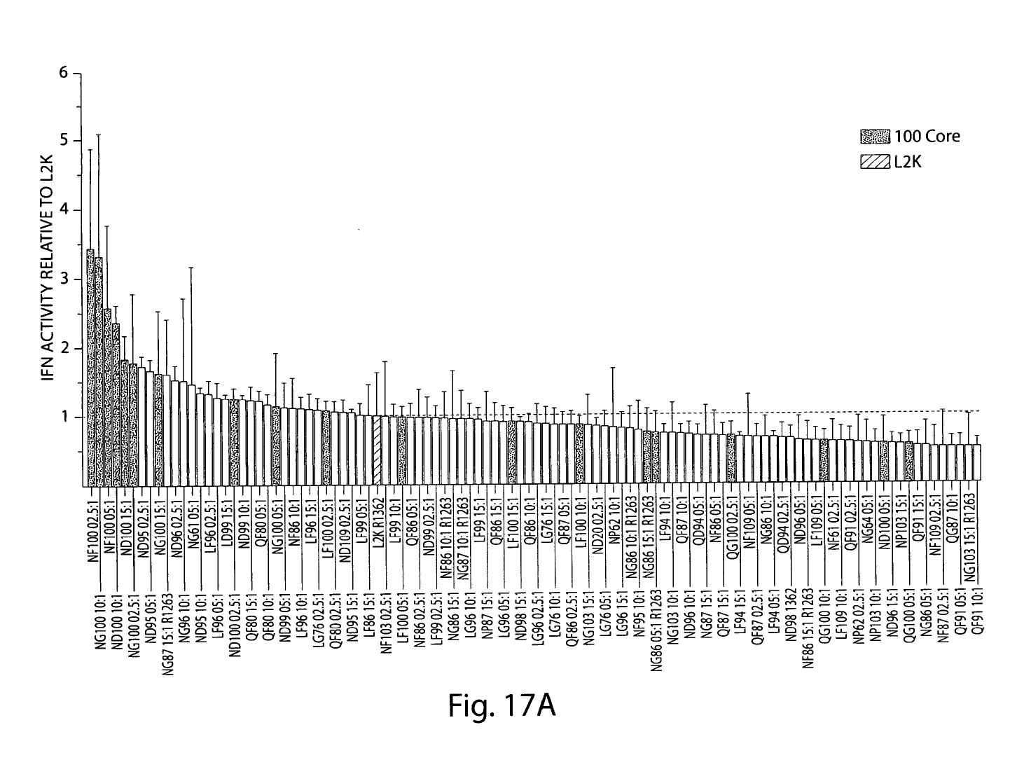

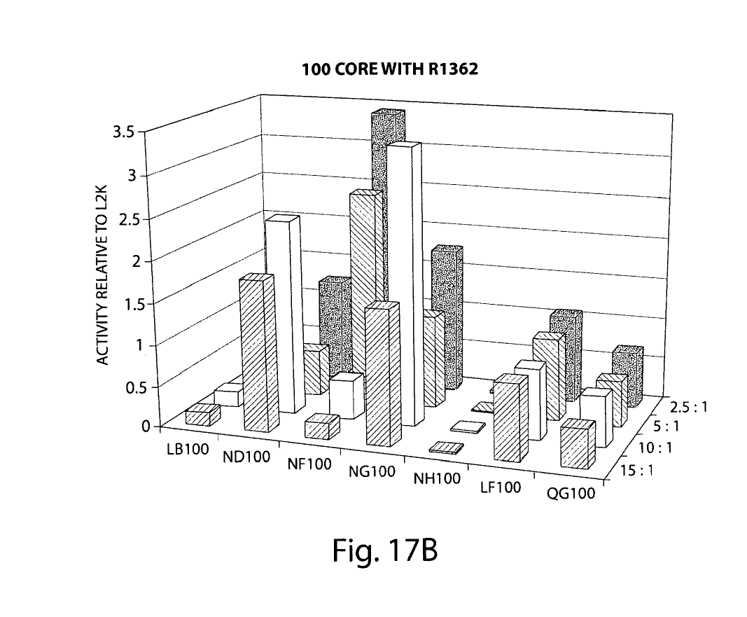

Many lipidoid compounds exhibited some level of activity. Of the almost 900 conditions tested, 106 combinations of lipid, RNA, and L/R ratio exhibited activity greater than half that of L2K (FIG. 17a); the 100-core amine is highly enriched in this subset. Of the 16 lipidoids that had activity equal to or greater than that of L2K at any L/R ratio (FIG. 10), four structures, including the top three lipidoid compounds, were derived from the 100-core amine (FIG. 10). Of the seven lipidoid 100-core products tested, five exhibited highly efficient delivery of R1362 (FIG. 17b). None of the 100-core compounds exhibited activity above baseline when complexed with R1263 (data not shown) indicating that Type I interferon activity in human PBMCs was due to efficient activation of TLR7 or TLR8.

### Synthesis and In Vitro Activity of Second Generation Lipidoids Based on the 100-Core

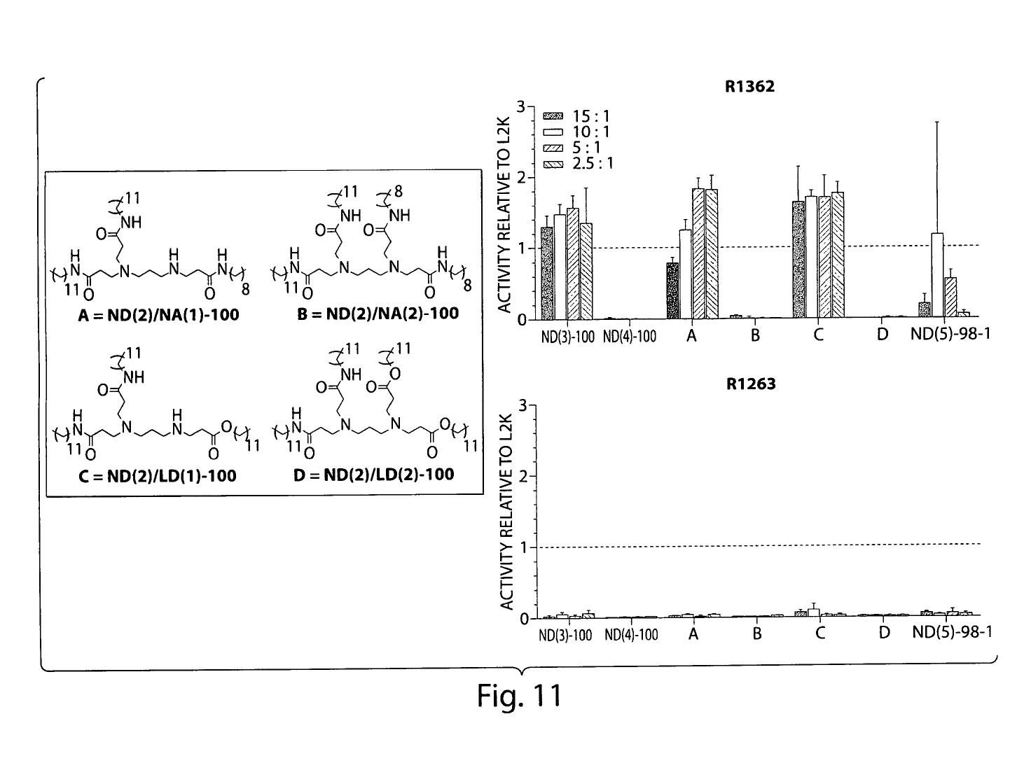

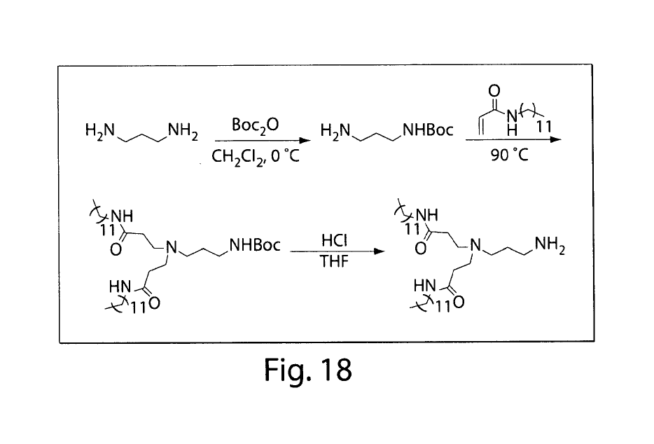

Focusing on the intrinsic activity of the 100-core structure, we designed new lipidoids for further screening. The 100-core has two primary amines that can be substituted with up to two alkyl tails each for a total of four possible substitutions. Long alkyl-chain tails were avoided due to decreased solubility. We focused on 3- and 4-tail versions of the 100-core as prior work with lipidoid-mediated siRNA delivery indicated that fully and (n−1)-substituted lipidoids exhibited the most efficient siRNA delivery.²⁶ Purified versions of ND100 and derivatives of ND100 with a mixture of ND and NA (further identified as “A” and “B”) or LD tails (further identified as “C” and “D”) (FIG. 11a) were synthesized by step-wise substitution of each primary amine (FIG. 18). Purified three-tailed lipidoids all exhibited high levels of is RNA delivery activity that was RNA-specific and greater than ND(5)-98-1 (FIG. 11b), a lipidoid material previously shown to have high capacity for siRNA delivery in vitro and in vivo.²⁶ However, 4-tailed lipidoids (ND(4)-100, B, and D) were not soluble in sodium acetate and therefore had low in vitro activity. These 100-core and second generation derivatives were further investigated for in vivo is RNA delivery.

Optimization of Lipidoid-RNA Nanoparticle Formulation for in vivo is RNA Delivery

To increase solubility and in vivo stability, lipidoids were formulated with poly(ethylene-glycol) (PEG) and cholesterol (Ch) and extruded through an 80 nm pore-size membrane to generate nanoparticles. RNA binding affinity was investigated by competitive binding with the RNA-specific fluorescent dye Ribogreen in the presence of Triton-X, a detergent that disrupts lipidoid-RNA binding. Second generation lipidoids A and B (ND/NA derivatives) bound R1362 more tightly than lipidoids C and D (ND/LD), with NC(3)-100 intermediate between the second generation lipidoids. 98-core materials bound more tightly to R1362 than did 100-core materials. At an L/R ratio of 11.5, nanoparticles in liquid form (prior to lyophilization) ranged in size from 70 nm to 300 nm with high encapsulation of RNA (Table 2).

TABLE 2
Representative characteristics of lipidoid-RNA nanoparticles.

| | Lipidoid: | | | | |
|---|---|---|---|---|---|
| | RNA | | Peak 1 | Peak 2 | Poly- |
| Lipidoid | ratio | Formulation | (nm) | (nm) | dispersity |
| ND(5)-98-1 | 15:1 | lyophilized | | 2768 | 0.400 |
| NC(5)-98-1 | 15:1 | lyophilized | 165 | 1507 | 0.900 |
| NC(3)-100 | 15:1 | lyophilized | 264 | | 0.400 |
| ND(3)-100 | 15:1 | lyophilized | 146 | 707 | 0.756 |
| ND(4)-100 | 15:1 | lyophilized | 87 | 659 | 1.000 |
| B | 15:1 | lyophilized | 83 | 560 | 1.000 |
| D | 15:1 | lyophilized | 144 | 1160 | 0.798 |
| B | 10:1 | lyophilized | | 2946 | 1.000 |
| D | 10:1 | lyophilized | 73 | 1209 | 0.620 |
| B | 15:1 | liquid | 69 | | 0.238 |
| D | 15:1 | liquid | 143 | | 0.400 |

Lipidoid nanoparticles were formulated at 15:1 or 10:1 ratios and either dialyzed or resuspended after lyophilization for in vivo injection. Particle sizes were measured by dynamic light scattering, and the size(s) at peak intensity are indicated.

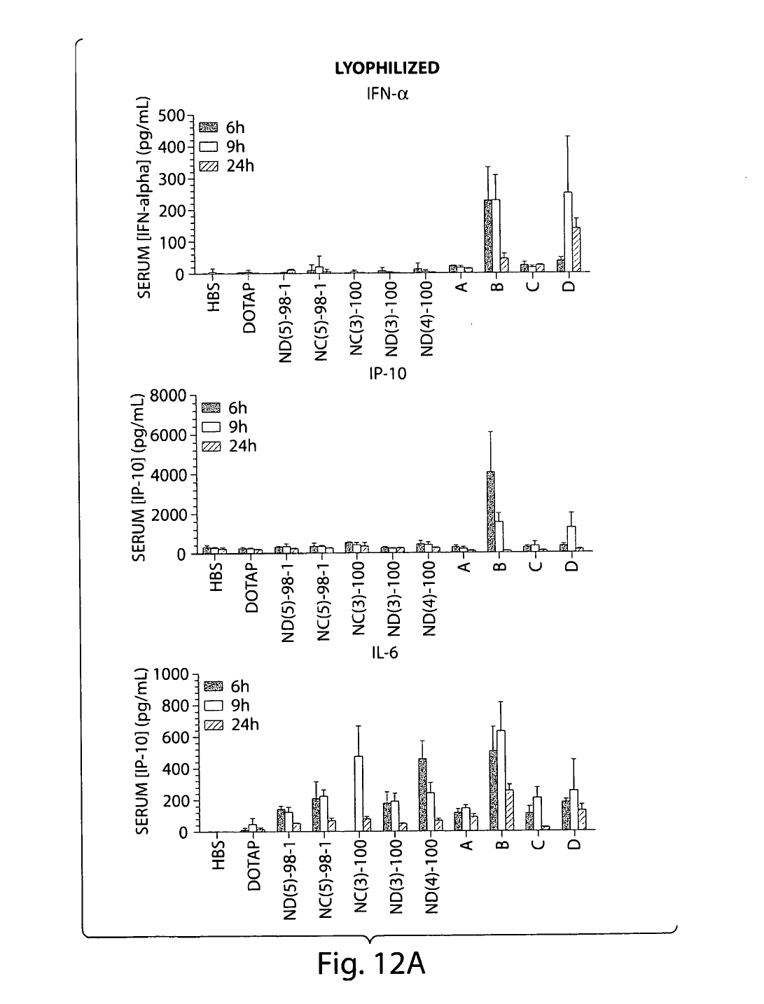

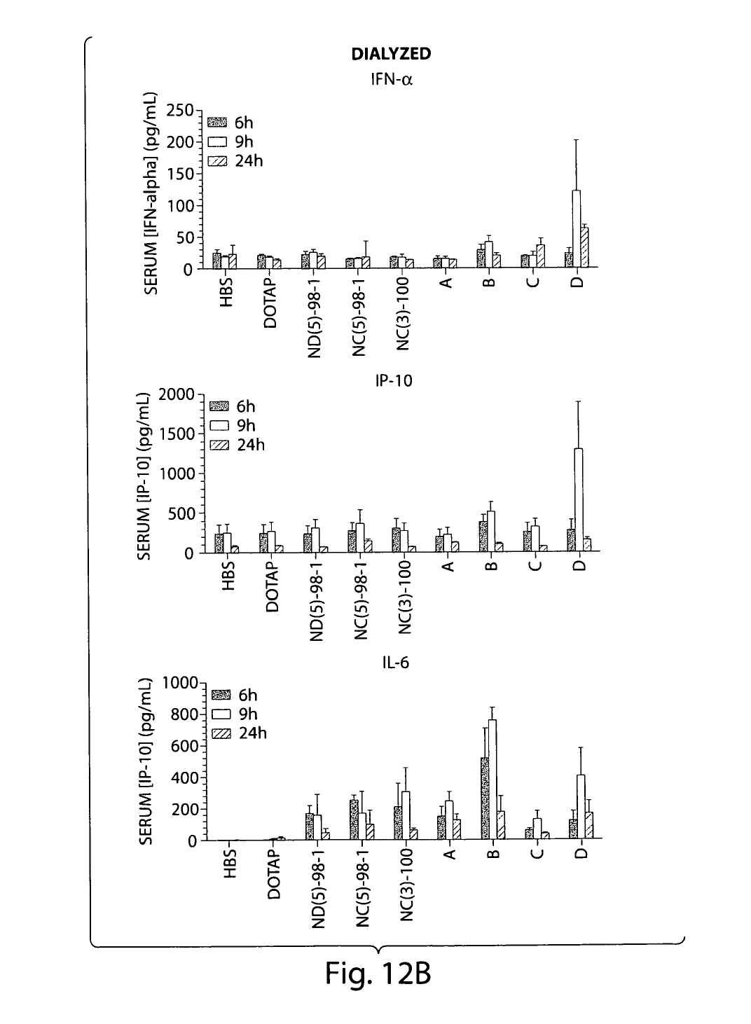

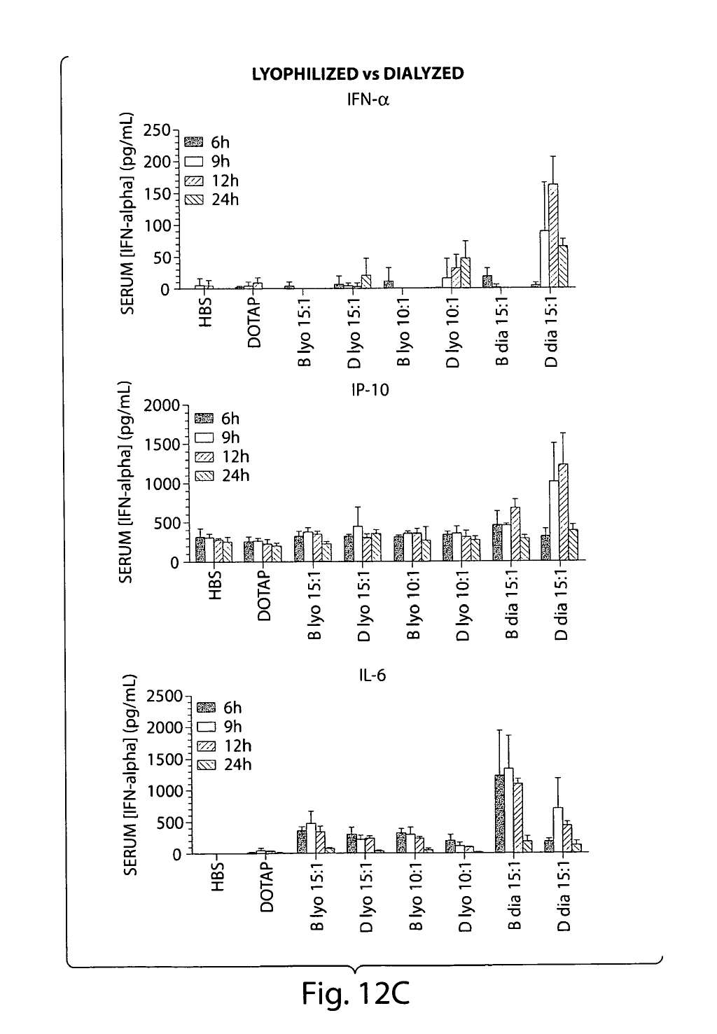

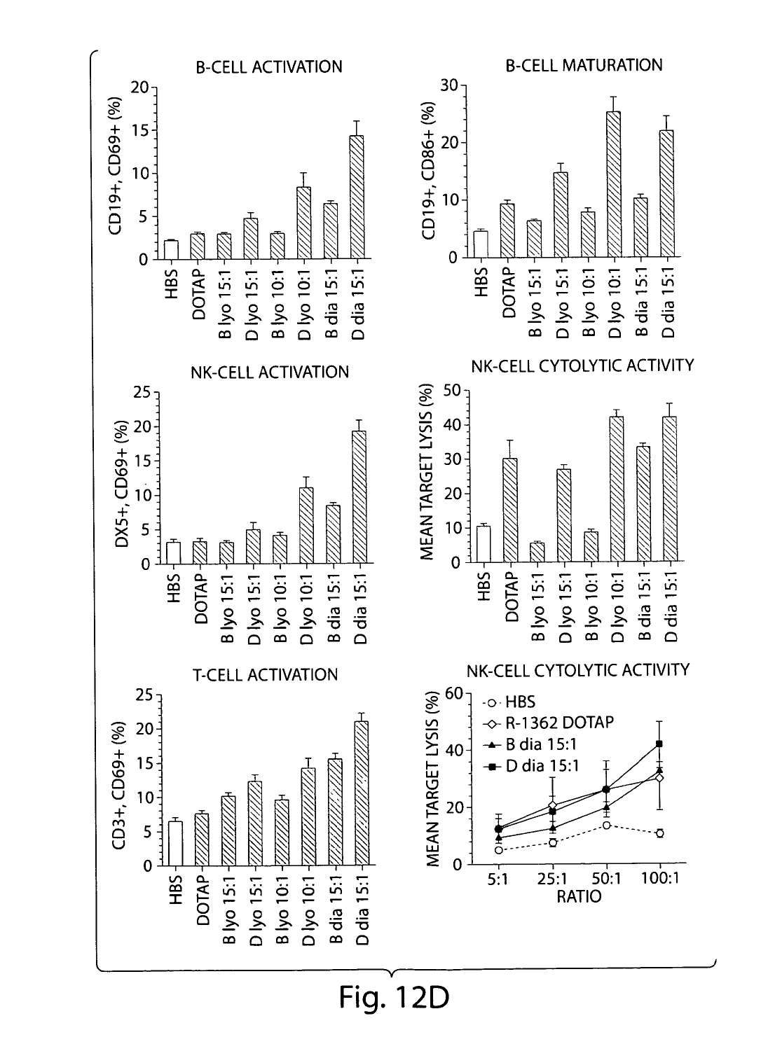

To investigate the ability of lipidoids to deliver is RNA in vivo, lyophilized and dialyzed versions of lipidoid-RNA nanoparticles encapsulating R1362 at a L/R ratio of 11.5 were injected subcutaneously in BALB/c mice. The time-course of innate immune cytokine signaling was monitored over 24 hours (FIG. 12a-c) and immune-cell activation in the spleen was assayed at 24 hours (FIG. 12d). The response to lipidoid-RNA complexes was compared to baseline (HBSS control injections) and to complexes of R1362 and DOTAP, a commercially-available cationic lipid commonly used for gene delivery. The second generation B and D lipidoids (both 4-tail versions) in particular exhibited high potential for is RNA delivery. Other lipidoids based on the 98-core were not as active as the 100-core materials. Compared to DOTAP-mediated delivery, lipidoid D induced, on average, up to 10-fold greater production of IFN-alpha, a marker of pDC acvitiy, and 5-fold greater production of IFN-gamma-induced protein 10 (IP-10) (FIG. 3a-b), while lipidoid B induced up to 50-fold greater IL-6 activation, a marker for myeloid dendritic cell mDC activation. T-cells, B-cells, and NK cells showed high activation in response to both B and D lipidoids (4-tailed lipidoids) (FIG. 12d). Compared to DOTAP-mediated delivery of R1362, lipidoid D activated on average 2.6 times as many CD3⁺ T-cells, eleven times as many CD19⁺ B-cells, and increased NK-mediated lysis of target cells over 3-fold (FIG. 12d).

Focusing on optimization of the B and D versions of the second generation 100-core lipidoids, nanoparticles were formulated with R1362 RNA at 10:1 and 15:1 L/R ratios and either dialyzed or lyophilized to remove ethanol. Freshly dialyzed liquid formulations at a 15:1 L/R ratio had the highest activity for cytokine induction (FIG. 12c). Again, dialyzed lipidoid D nanoparticles exhibited highest activation of IFN-alpha and IP-10, up to 18-fold and 4.5-fold compared to DOTAP, respectively, while dialyzed lipidoid B nanoaprticles induced highest activation of IL-6 (FIG. 12c) with a 30-fold increase over DOTAP delivery. Formulation B also activated high levels of TNF-a, IL-5, and GM-CSF (data not shown). Splenic T-cells, B-cells, and NK cells were activated by both B and D in dialyzed formulations (FIG. 12d). CD19⁺ B-cells in particular responded highly to lipidoid D in both lyophilized and dialyzed formulations. All lyophilized lipidoid particles were found to have a heterogeneous size distribution (into the micron range) and reduced stability after resuspension in HBSS (Table 2), but dialyzed liquid formulations were in the true nanoparticle size range (below 200 nm), with lipidoid D particles (143 nm) being slightly larger than lipidoid B nanoparticles (69 nm).

Lipidoid-RNA nanoparticles based on the B and D lipidoids were formulated with either R1362 or R1263 at 15:1 L/R ratio. Nanoparticles sizes of formulations of B and D were not dependent on encapsulated RNA and were stable in size over the course of 1 month (Table 3) with some increased in aggregation observed with lipidoid D. Mixing chicken ovalbumin (Ova) protein antigen with lipidoid-nanoparticles did not alter size-distributions.

TABLE 3
Size stability of optimized lipidoid-RNA nanoparticles formulation.

| Lipid- | | Average | Peak (nm) | |
|---|---|---|---|---|
| oid | RNA | (nm) | Intensity (%) | Time |
| B | R1362 | 140.0 | 162.2 | Immediately |
| | R1263 | 125.0 | 171 | after |
| D | R1362 | 174.0 | 199.8 | formulation |
| | R1263 | 172.2 | 258 | |
| B | R1362 | 124.0 | 188 (100%) | One month |
| | R1263 | 133.0 | 188 (100%) | after |
| | R1362 + Ova | 122.0 | 168 (100%) | formulation |
| | R1263 + Ova | 136.0 | 183 (100%) | |
| D | R1362 | 130.0 | 41 (23%), 278 (75%) | |
| | R1263 | 179.0 | 35 (17%), 149 (28%), | |
| | | | 582 (54%) | |
| | R1362 + Ova | 133.0 | 58 (34%), 314 (63%) | |
| | R1263 + Ova | 182.0 | 32 (14%), 142 (30%), | |
| | | | 686 (53%) | |

Particles sizes were measured by dynamic light scattering immediately after formulation or after 1 month of storage at room temperature. Average particle size and size of peak intensity are reported. If multiple size peaks were detected, the relative intensity is indicated by (%). Chicken ovalbumin protein antigen (Ova) was mixed with nanoparticles at a 2:1 ratio (w/w) of RNA:Ova immediately prior to measurement.

Characterization of Innate Immune Activation In Vivo with Lipidoid-RNA Nanoparticles

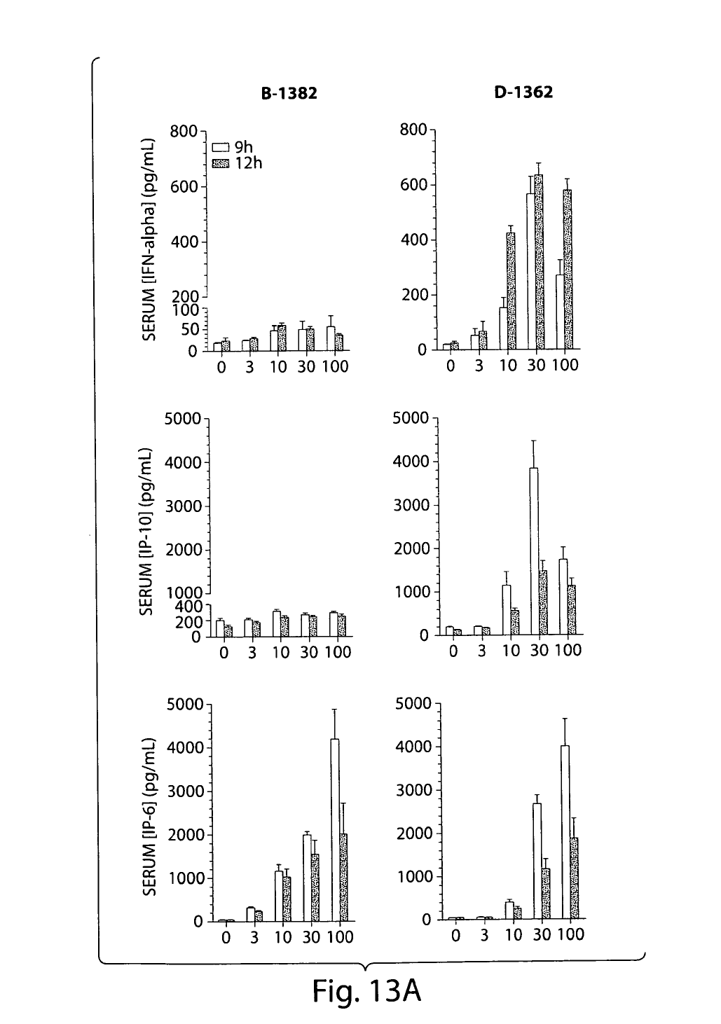

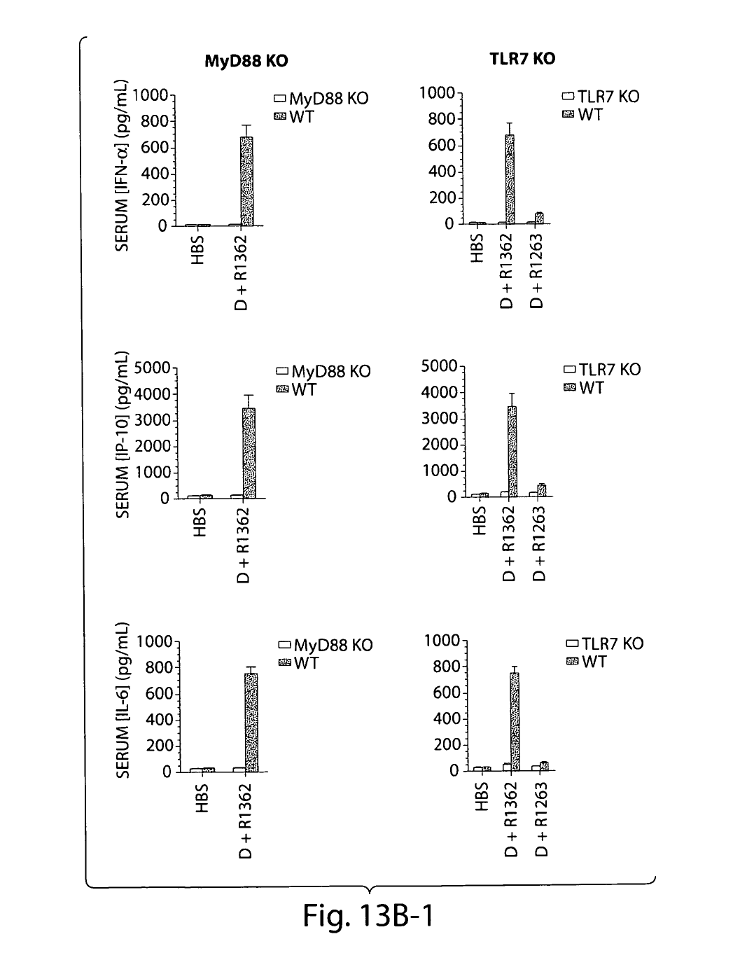

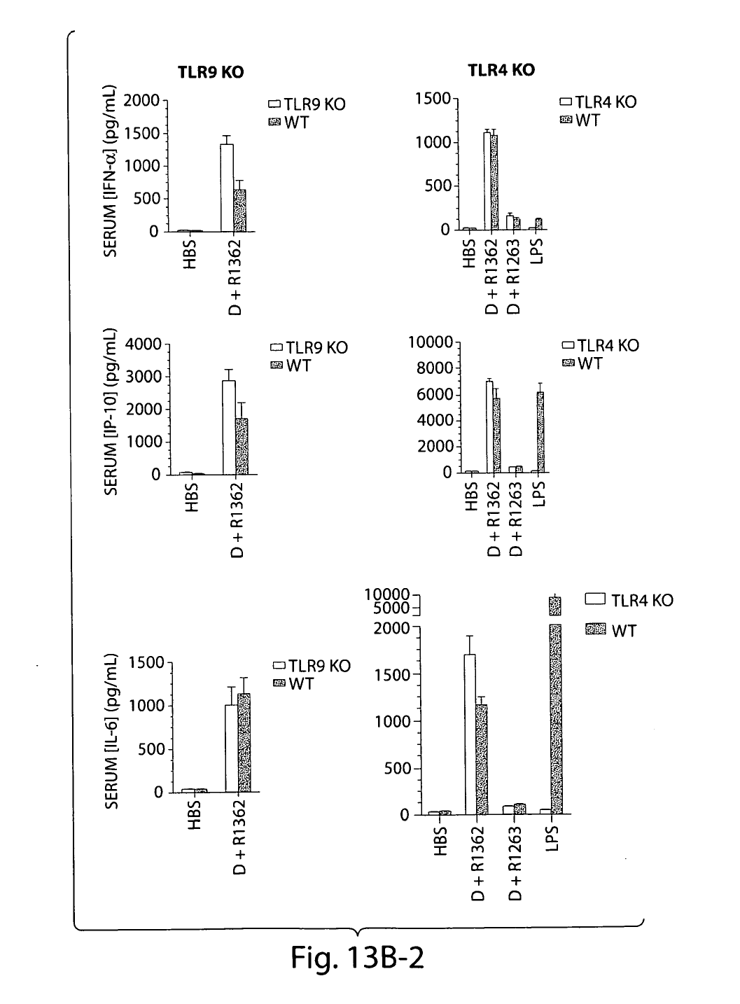

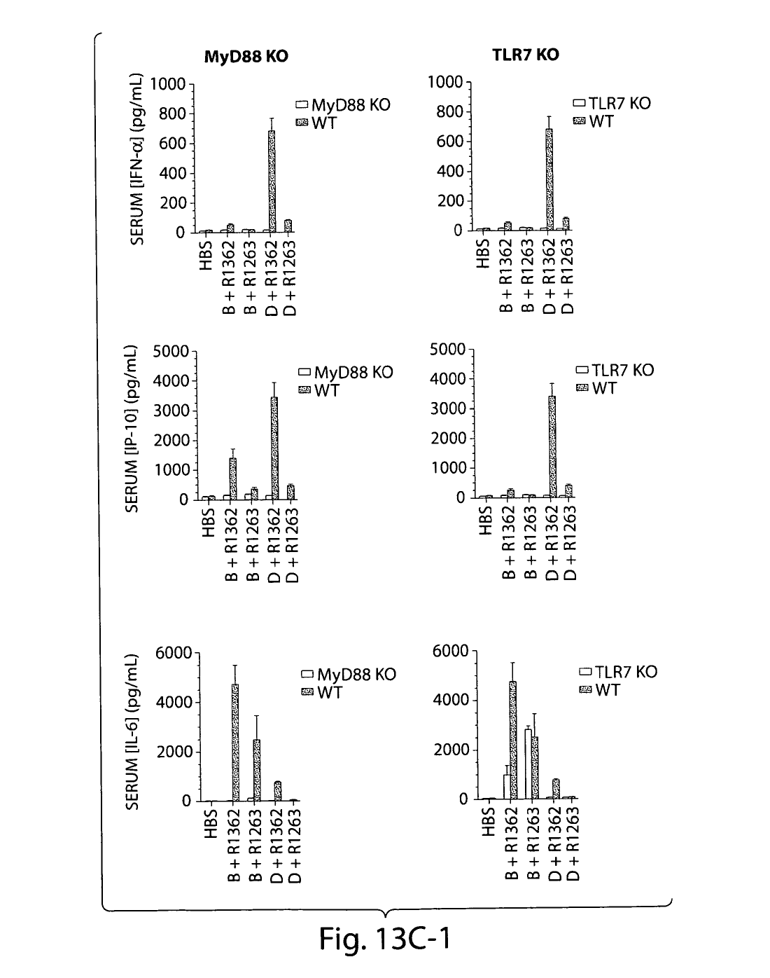

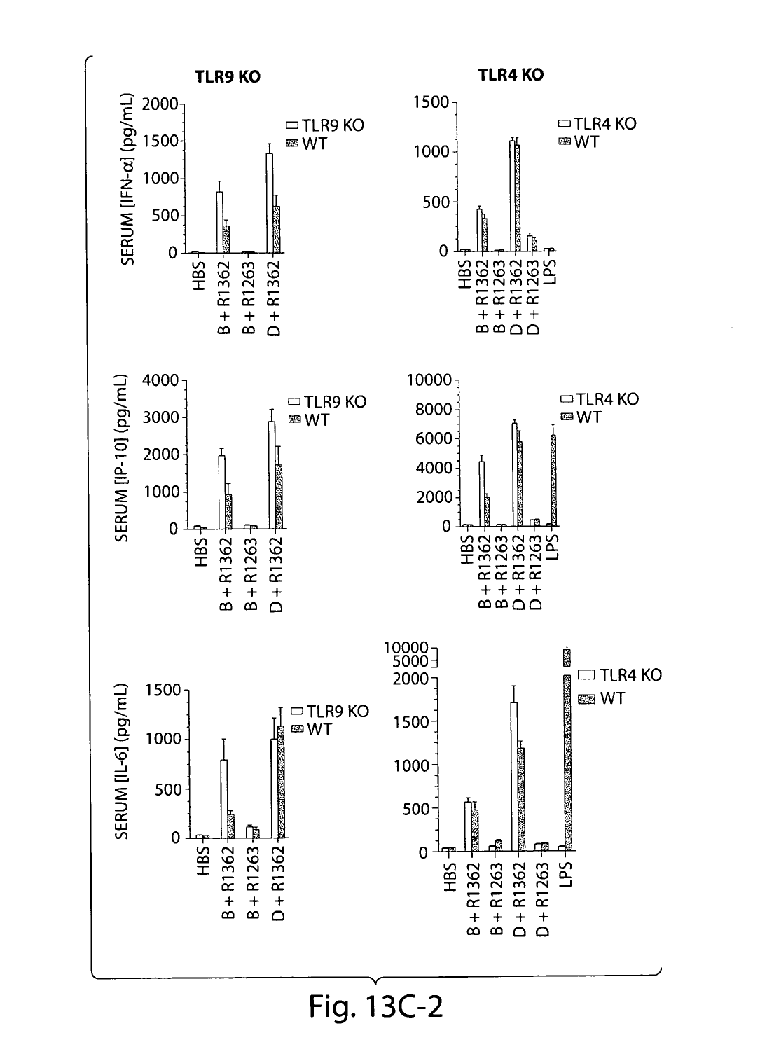

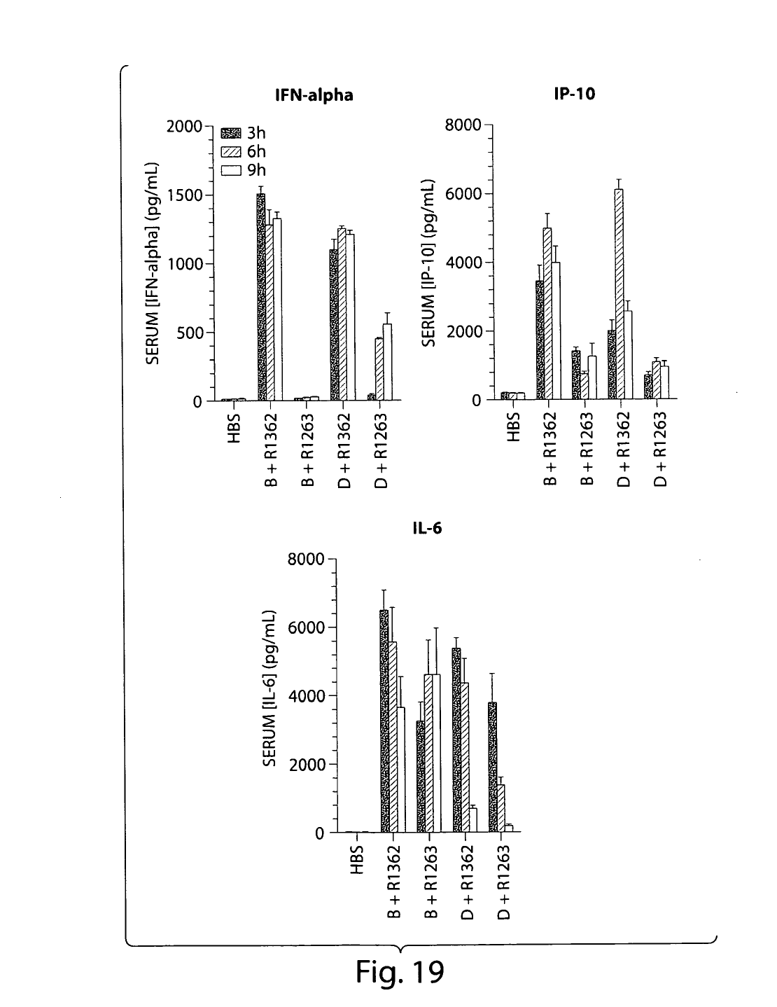

To investigate the RNA-specific nature of innate immune responses, we investigated profiles of innate immune activation in 129sv mice following SC administration of 3 μg to 100 μg of R1362 RNA. There was a clear difference in potency for activation of IFN-alpha and IP-10, with lipidoid D nanoparticles activating much greater amounts of these two markers of pDC activation (FIG. 13a). To investigate sequence specificity in innate immune activation, we compared lipidoid B and D nanoparticles formulated with R1362 to those formulated with R1263. After IV injection, a sharp sequence-specific contrast in stimulation of serum IFN-alpha and IP-10 was observed between nanoparticles containing the strong TLR7/8 agonist R1362 and the weak agonist R1263 (FIG. 19). Activation of IFN-alpha by nanoparticles with R1263 was higher for lipidoid D formulations than for lipidoid B. Both R1362- and R1263-containing nanoparticles induced IL-6 production that was greater for R1362 RNA than R1263 and greater with lipidoid B than lipidoid D.

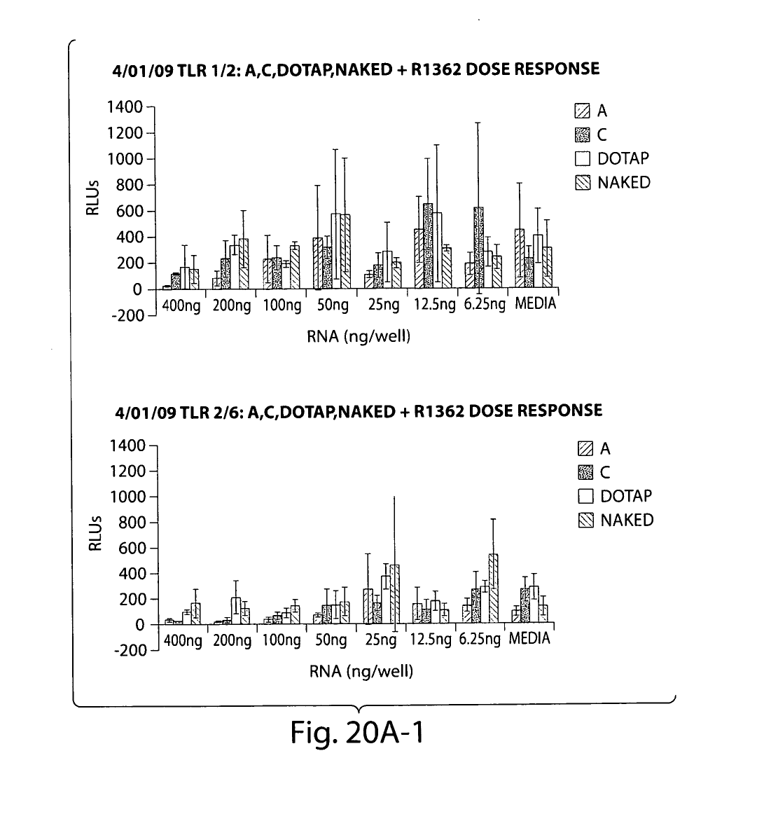

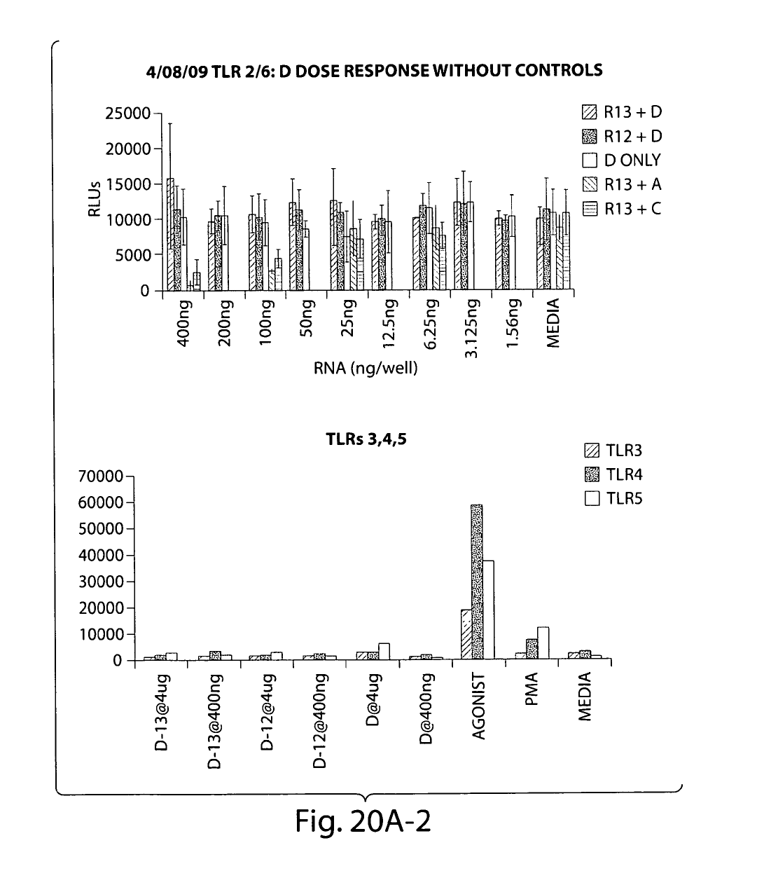

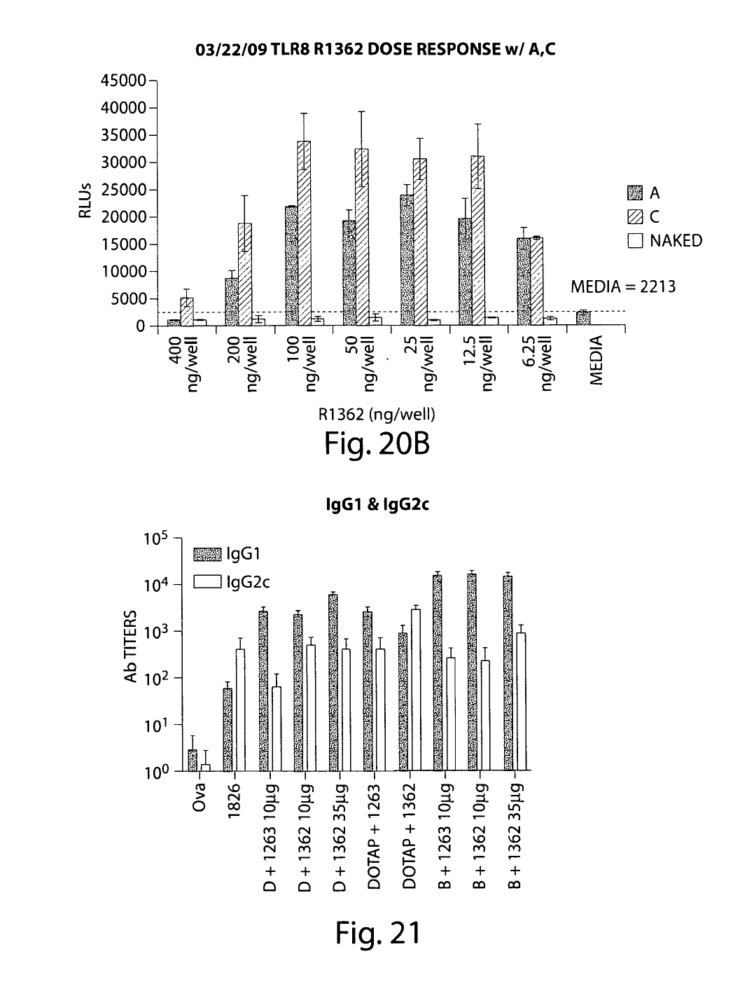

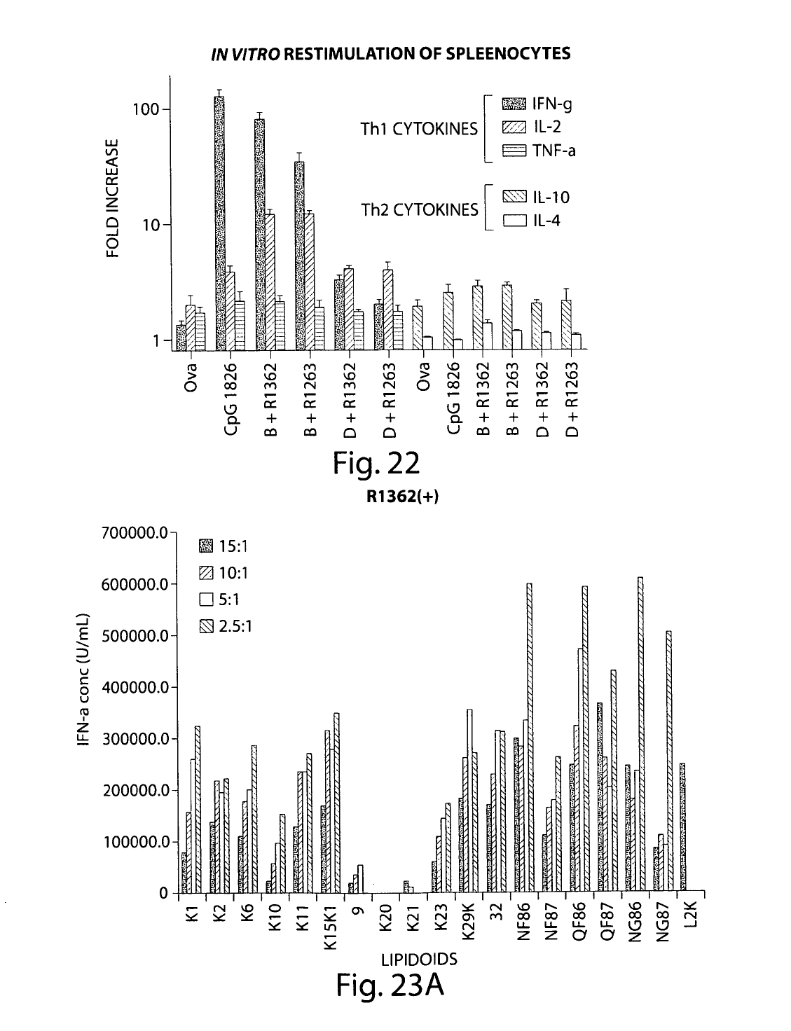

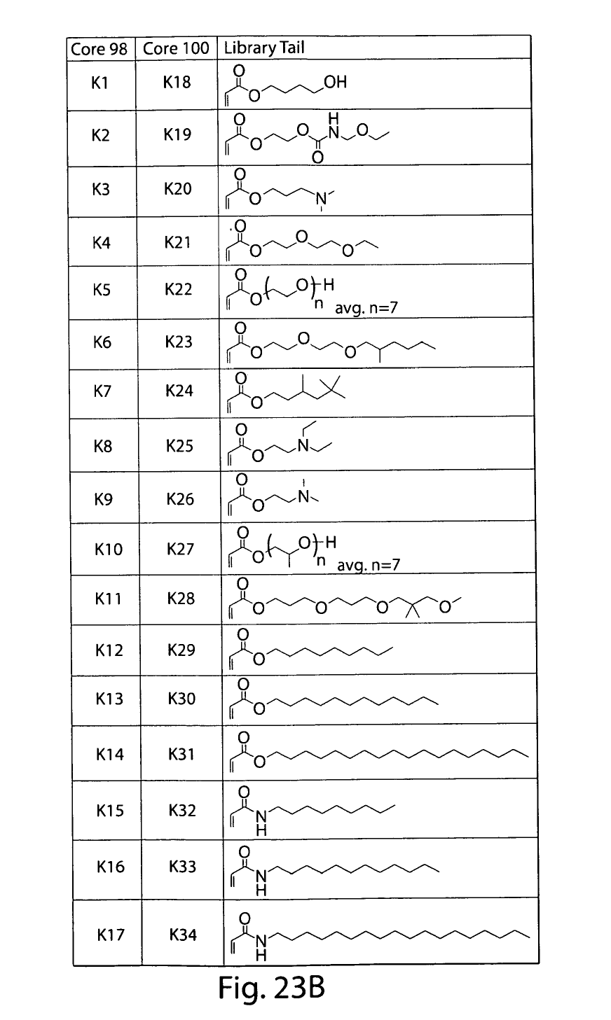

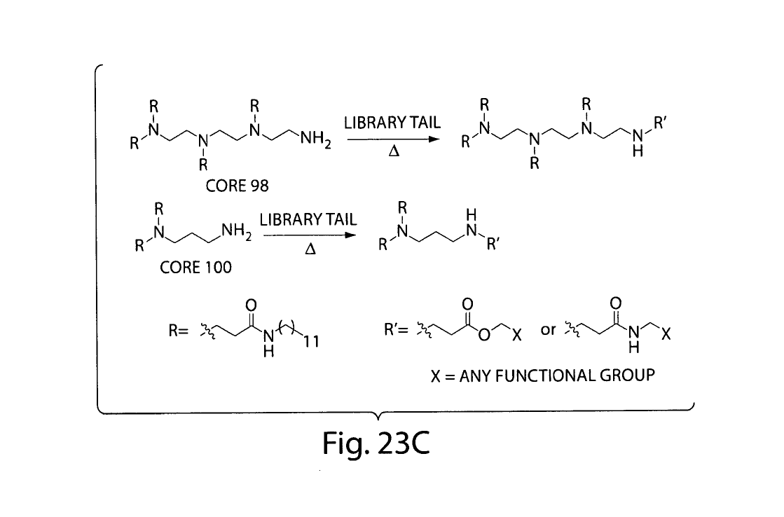

The potential for non-TLR7/8-mediated activation of these cytokines was explored by IV injections of 75 μg RNA into knockout mice that are MyD88^{-/-}, TLR7^{-/-}, TLR9^{-/-}, TLR4^{-/-}, or wild-type (WT) on matched backgrounds (FIG. 13b). Production of IFN-alpha, IP-10, and IL-6 were all dependent on MyD88 signaling, but only IFN-alpha and IP-10 production were dependent upon TLR7. L-6 production in response to B+R1362 nanoparticles was decreased but present in TLR7^{-/-} mice, but IL-6 production in response to B+1263 nanoparticles was not effected by loss of TL7 function. IL-6 production in response to lipidoid D nanoparticles was RNA-specific and completely dependent upon TLR7. Neither TLR9 nor TLR4 were required for cytokine production. Additionally, no bacterial endotoxin was detected by LAL assay in any batches of nanoparticles (data not shown). Further, HEK293T cells stably expressing human TLRs 2,3,4,5, and 6 were incubated with lipidoid-RNA complexes without observation of any TLR activity above background (FIG. 20). However, HEK293T stably transfected with either TLR8 exhibited dose-dependent activation by lipidoids complexed with R1362 RNA (FIG. 20).

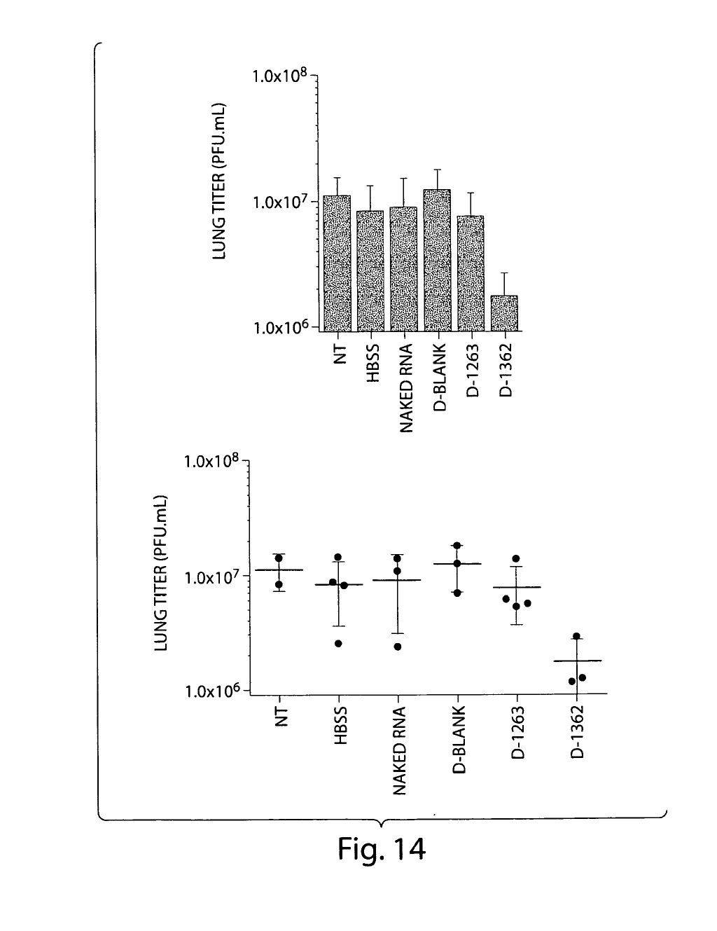

The biological activity of the type I interferons induced by lipidoid-RNA nanoparticles was examined using an established in vivo model of influenza infection that is sensitive to is RNA activity.²⁸ As lipidoid D nanoparticles exhibited the greatest amount of IFN-alpha activation, these lipidoids were injected subcutaneously into 129Sv mice. After 24 hours, mice were challenged intranasally with influenza A/PR8 virus, and lung viral titer was determined after another 24 hours of incubation. Prophylactic protection against infection was only observed for R1362 RNA complexed with lipidoid D formulations, while naked RNA, D+R1263, or the D lipidoid nanoparticles without RNA did not induce an antiviral state (FIG. 14).

### Lipidoid-RNA Nanoparticles Increase Adaptive Immune Responses to Protein Antigens

Lipidoid-RNA nanoparticles were mixed with chicken ovalbumin protein antigen (Ova) to investigate the adjuvant activity of lipidoid-mediated delivery of is RNA agonists. C57Bl/6 mice were vaccinated with intramuscular injections of Ova protein without adjuvant, lipidoid nanoparticles mixed with Ova protein, or CpG 1826 ODN, a TLR9 agonist, mixed with Ova protein.

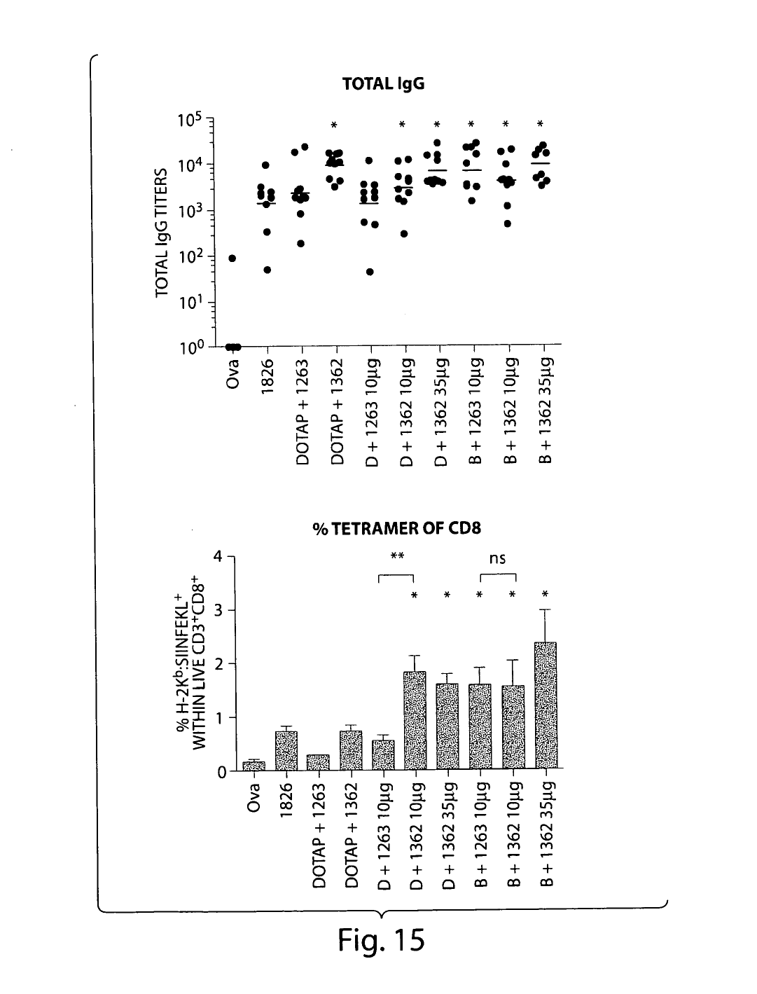

Vaccination with lipidoid-RNA nanoparticles as an adjuvant increased humoral immune responses by 3 to 4 orders of magnitude compared vaccination with protein alone. An significant increase in total IgG was observed for B lipidoids with both R1362 and R1263, but for D lipidoids only vaccination with R1362 RNA resulted in statistically significant greater levels of IgG antibody (FIG. 15a). Vaccination with lipidoid particles resulted in an increase in both IgG1 and IgG2a subclasses compared to protein alone (FIG. 21). While CpG adjuvant resulted in a greater proportion of IgG2c, lipidoid adjuvants preserved the relatively greater IgG1 bias observed with protein vaccination alone.

Vaccination with lipidoid-RNA nanoparticles also greatly stimulated cell-mediated immune responses. Both B+1362 and D+1362 nanoparticles induced greater numbers of splenic antigen-specific CD8⁺ T-cells than with CpG 1826 ODN, and all lipidoid formulations increased antigen-specific CD8⁺ T-cells to levels greater than that with pure Ova protein vaccination (FIG. 15b). With D lipidoid nanoparticles, the increase in percentage of reactive CD8+ positive T-cells was significantly greater with the R1362 RNA than the R1263. With B lipidoids, the percentage of reactive CD8+ positive T-cells was large but not significantly different for either RNA. Restimulation in vitro of splenocytes with Ova protein resulted in large increases in the secretion of Th1-biasing cytokines IFN-gamma and IL-2. Only minor increases were detected in the Th2-associated cytokines IL-10 and IL-4.

### Discussion

The controlled drug delivery of ssRNA agonists to cell types expressing TLR7 and TLR8 has the potential for therapeutic activation of the innate immune system. We synthesized a library of cationic lipidoid materials²⁶ and screened this library in a high throughput manner for the ability to deliver is RNA in vitro using the ssRNA R1362 sequence that engages both TLR7 and TLR8 in a variety of immune cells and across multiple species including human and mouse.²¹ Screening revealed a large number of promising candidate lipidoid materials for delivery of is RNA (FIG. 10). The majority of delivery function seems to be intrinsic to the core amine-containing structure. The population of lipidoids achieving efficient is RNA delivery was highly enriched for a specific diamine core, 100, that was present in the top 3 compounds tested in the screen. While other lipidoid compounds also showed promise as is RNA delivery agents (FIGS. 10c and 17a), we focused on the 100-core due to its R1362-specific potency and activity across a variety of related lipidoid compounds with variable tail-lengths and chemistries. To control for non-specific activation, a secondary screening with the control ssRNA R1263 sequence²¹ was performed. Some materials were equally active with both the R1362 and R1263 sequences (FIG. 17a) such as the 86 and 87 core. Screening was performed on unpurified reactants that may contain a crude mixture of fully or incompletely substituted lipidoids. Because this screening environment is completely aqueous, selection is biased against hydrophobic materials such as fully substituted compounds. Thus, most screening activity is likely due to incompletely substituted lipidoid compounds.

Based upon initial screening results, new 100-core second generation lipidoids incorporating short chain lengths and mixed alkyl chains were developed for further testing in vitro and in vivo (FIG. 11a). Purification of 100-core materials into 3-tailed and 4-tailed components confirmed that in vitro delivery of is RNA to TLR7 and TLR8 in human PBMCs is due to incompletely substituted lipidoids that are water soluble (FIG. 11b). However, formulation of lipidoid nanoparticles for in vivo delivery can be done in ethanol whereby fully substituted (i.e., 4-tail) hydrophobic compounds can be dissolved thus avoiding these limitations. Indeed, whereas in vitro testing favors 3-tailed compounds (FIG. 11b), in vivo formulated nanoparticles were most active with fully-substituted 4-tail lipidoids (FIG. 12). Nanoparticles for injection in vivo were formulated with PEG-ceramide, which can increase circulation time and prevent non-specific uptake, and cholesterol, which stabilizes liposome structure. Hydrophobic lipidoids may have stronger interactions with these components as well as provide more order by favoring segregation at the molecular level into hydrophobic and hydrophilic domains. Removal of ethanol prior to injection can be accomplished either by dialysis or by lyophilization. Previous work indicated that lyophilization could increase the is RNA delivery characteristics of the ND98 lipidoid,²⁸ however, lyophilized formulations of the 100-core lipidoids had more heterogeneous size.

### Methods

### RNA

RNAs were fully phosphorothioate-modified, 20-base, single-stranded RNA synthesized by Coley Pharmaceuticals with sequences as previously described²¹: R1362 [5′-UUGUUGUUGUUGUUGUUGUU-3′] and R1263 [5′-GCCACCGAGCCGAAGGCACC-3′].

### Combinatorial Lipidoid Synthesis

Lipidoids²⁶ were synthesized in a combinatorial fashion as depicted in FIG. 2 in solvent-free conditions by reacting primary and secondary amine-containing cores (FIG. 1, right) with alkyl-acrylate or alkyl-acrylamide (FIG. 1, left) tails at a high tail-to-core monomer ratio to drive synthesis of fully (n)-substituted lipidoids. Lipidoid products were purified of un-reacted core and side-chain reactants resulting in crude mixtures of fully and incompletely-substituted lipidoids by silica gel chromatography. Some alkyl-acrylate-tail lipidoids were further reacted with methyl-iodide (FIG. 16) to form quaternized amines with a permanent positive charge. Second-generation lipidoids (FIG. 11a) were synthesized in a four-step process (FIG. 11b). The 100-core diamine was mono-protected by reacting 10× molar excess pure diamine with di-tert-butyl dicarbonate (Boc₂O). ND tails were reacted with the free primary amine in excess prior to deprotection and regeneration of the opposite primary amine resulting in ND(2)-100. ND(2)-100 was further reacted with NA or LD tails and purified into 3-tail or 4-tail derivatives, which have been renamed lipidoids A-D for clarification. Nomenclature reflects alkyl tail linkage (acrylate=L, acrylamide=N), alkyl tail carbon-chain length (A=9, B=10, D=12, F=14, G=15, P=16, H=18 carbons), and amine-containing core. Quaternized core-amines are further referred to with a Q designation instead of L. Purified lipidoids includes number of tails in parenthesis ( ) following tail name. A complete list of crude lipidoids screened is found in Table 1.

### High-Throughput Screening for Lipidoid-Mediated is RNA Delivery

Donor-blind buffy-coat packs were obtained from the Massachusetts General Hospital blood bank. Peripheral blood mononuclear cells (PBMC) were isolated by Ficoll-Paque Plus (Amersham Biosciences) density centrifugation. PBMCs were resuspended in supplemented RPMI (RPMI 1640 medium with 10% FCS, 1 mM MEM sodium pyruvate, 10 mM HEPES, and 100 U/mL penicillin/streptomycin) and plated at 5×10⁵ cells/well in 175 μL in 96-well tissue culture plates. Lipofectamine 2000 (L2K) (Invitrogen) was used as a positive control for transfection of RNA according to manufacturers protocols and to normalize interferon responses across different donors.

Crude or purified lipidoid products were dissolved to 0.5 mg/mL in 25 mM sodium acetate, pH5, followed by brief sonication. For lipidoids with poor solubility, up to 10% DMSO was added to stock lipidoid solutions. RNA was dissolved to 50 μg/mL in sodium acetate. Lipidoids were arrayed in 96-well round-bottom reaction plates and mixed at 15, 10, 5, and 2.5:1 mass ratios of lipid to RNA for 80 μL total volume. Complexes were diluted 120 μL RPMI media after 20 minutes incubation at room temperature to allow for nanoparticle complexes to form. In quadruplicate, 25 μL of diluted complexes were added to PBMCs for a final RNA concentration of 200 ng RNA per well in 200 μL media (1 μg/mL˜140 nM). After 16-20 hours of incubation, PBMC cultures were centrifuged at 400 RCF for 10 minutes, and supernatants were stored at −80° C. for later quantification.

Type I interferon activity was quantified using a HT-compatible cell-based detection assay as previously described.²⁹ Briefly, 293T-ISRE-RFP cells were incubated with 50 μL PBMC supernatant overnight prior to HT-FACS analysis of red fluorescence. Recombinant human interferon alpha (hIFN-a) (PBL Laboratories) serially diluted in supplemented RPMI was used as a standard, and type I interferon activity of each screening well was normalized to activity from L2K transfections.

### Formulation and Characterization of Lipidoid-RNA Nanoparticles for Injection

Purified lipidoid was dissolved to 120 mg/mL in ethanol. Cholesterol (Ch) (Sigma Aldrich, St Louis, Mo.) was dissolved to 25 mg/mL in ethanol. N-palmitoyl-sphingosine-1-[succinyl(methoxypolyethylene glycol)2000] (C16 mPEG 2000 ceramide) (PEG) (Avanti Polar Lipids, Alabaster, Ala.) was dissolved to 100 mg/mL in ethanol. Lipidoid, Ch, and PEG were combined at a 15:0.8:7 mass ratio (L:C:P), vortexed briefly, and diluted in a mixture of ethanol and 200 mM sodium acetate (with 16.67 mg/mL sucrose for lyophilization) for a final lipidoid concentration of 7.5 mg/mL in 35% ethanol, 65% NaAc. RNAs were resuspended in water to 10 mg/mL and diluted to 35% ethanol. Lipidoid/Ch/PEG were added to diluted RNA at a 15, 11.5, or 10:1 mass ratio (L:R) and vortexed for 20 minutes to allow complexes to form. Complexed lipidoid-RNA nanoparticles were extruded once through a double 200 nm membrane and then twice through a double 80 nm membrane (Whatman, Florham Park, N.J.) on a Northern Lipids (British Columbia, Canada) extrusion system at 40° C. To remove ethanol prior to injection, nanoparticles were dialyzed in a Slide-A-Lyzer 3500 MWCO dialysis cassette (Pierce Biotech) against HBSS. For lyophilization, 10 mg of sucrose was added per mL of extruded complexes prior to freezing at −80° C. for >2 hours followed by >1 day lyophilization.

For quantification and encapsulation efficiency of RNA, a 50 μL sample of nanoparticles was diluted 200-fold in Tris-EDTA buffer (TE), mixed with either 50 μL of TE buffer or 50 μL of 2% Triton-X-100 (T-X) in TE, and incubated with 100 μL Quant-It Ribogreen reagent (Invitrogen) for 20 minutes at 37° C. in a 96-well black plate. Total fluorescence in the presence of T-X was compared to a standard curve of RNA diluted in TE to determine total RNA concentration; RNA encapsulation efficiency was determined by the ratio of fluorescence signal without T-X and with T-X. Nanoparticle size and zeta-potential of formulated lipidoid-RNA nanoparticles was assayed by dynamic light scattering using a Zeta-PALS instrument (Brookhaven Instruments) after 1/50 dilution in PBS or with a Mastersizer instrument (Malvern Instruments) after dilution in HBSS.

### In Vivo Characterization of Innate Immune Responses

Animal studies were conducted at Coley Pharmaceuticals (Ottowa, ON, Canada) under the approval of the institutional care committees and in accordance with the guidelines set forth by the Canadian Council on Animal Care. Formulated lipidoid-RNA nanoparticles were resuspended or diluted in HBSS prior to injection under isofluorane anesthesia. Innate immune responses of multiple lipidoid formulations with R1362 RNA were compared after subcutaneous (SC) injections in BALB/c mice. To further investigate RNA-specific immune responses, nanoparticles were injected SC at increasing doses or intravenously (IV) into 129sv mice. TLR-mediated responses were investigated following IV injection in C57Bl/6, C57Bl/6 TLR7^{-/-}, C57Bl/6 TLR9^{-/-}, C57Bl/6 MyD88^{-/-}, C3H, or C3H TLR4^{-/-}. As a control, DOTAP (1,2-dioleoyloxy-3-(trimethylammonium)propane) (Roche) was complexed with RNA at a 2:1 weight ratio (L:R). Blood samples for serum isolation were taken by direct cardiac puncture on heparin at indicated timepoints. Serum samples were analyzed by ELISA with commercially available antibodies (IFN-alpha, PBL Labs) (IP-10, BD Pharmingen) or 10-plex Luminex technology (BioSource International). Spleenocytes were also harvested for surface expression of activation markers by staining with anti-CD69-FITC, anti-CD86-APC, anti-CD3-PE/Cy7, anti-CD19-ECD, and anti-DX5-PE.

### REFERENCES

1. Iwasaki, A. & Medzhitov, R. Toll-like receptor control of the adaptive immune responses. Nat Immunol 5, 987-995 (2004).
2. Zhang, S. Y. et al. Human Toll-like receptor-dependent induction of interferons in protective immunity to viruses. Immunol Rev 220, 225-236 (2007).
3. Krieg, A. M. & Vollmer, J. Toll-like receptors 7, 8, and 9: linking innate immunity to autoimmunity. Immunol Rev 220, 251-269 (2007).
4. Jarrossay, D., Napolitani, G., Colonna, M., Sallusto, F. & Lanzavecchia, A. Specialization and complementarity in microbial molecule recognition by human myeloid and plasmacytoid dendritic cells. Eur J Immunol 31, 3388-3393 (2001).
5. Schon, M. P. & Schon, M. TLR7 and TLR8 as targets in cancer therapy. Oncogene 27, 190-199 (2008).
6. Marshak-Rothstein, A. Toll-like receptors in systemic autoimmune disease. Nat Rev Immunol 6, 823-835 (2006).
7. Pashine, A., Valiante, N. M. & Ulmer, J. B. Targeting the innate immune response with improved vaccine adjuvants. Nat Med 11, S63-68 (2005).
8. Steinman, R. M. & Banchereau, J. Taking dendritic cells into medicine. Nature 449, 419-426 (2007).
9. Hemmi, H. et al. Small anti-viral compounds activate immune cells via the TLR7 MyD88-dependent signaling pathway. Nat Immunol 3, 196-200 (2002).
10. Diebold, S. S., Kaisho, T., Hemmi, H., Akira, S. & Reis e Sousa, C. Innate antiviral responses by means of TLR7-mediated recognition of single-stranded RNA. Science 303, 1529-1531 (2004).
11. Heil, F. et al. Species-specific recognition of single-stranded RNA via toll-like receptor 7 and 8. Science 303, 1526-1529 (2004).
12. Lan, T. et al. Stabilized immune modulatory RNA compounds as agonists of Toll-like receptors 7 and 8. Proc Natl Acad Sci USA 104, 13750-13755 (2007).
13. Scheel, B. et al. Therapeutic anti-tumor immunity triggered by injections of immunostimulating single-stranded RNA. Eur J Immunol 36, 2807-2816 (2006).
14. Eberle, F. et al. Modifications in Small Interfering RNA That Separate Immunostimulation from RNA Interference. J Immunol 180, 3229-3237 (2008).
15. Sioud, M. Induction of inflammatory cytokines and interferon responses by double-stranded and single-stranded siRNAs is sequence-dependent and requires endosomal localization. J Mol Biol 348, 1079-1090 (2005).
16. Judge, A. D., Bola, G., Lee, A. C. & MacLachlan, I. Design of noninflammatory synthetic siRNA mediating potent gene silencing in vivo. Mol Ther 13, 494-505 (2006).
17. Morrissey, D. V. et al. Potent and persistent in vivo anti-HBV activity of chemically modified siRNAs. Nat Biotechnol 23, 1002-1007 (2005).
18. Robbins, M. et al. 2′-O-methyl-modified RNAs act as TLR7 antagonists. Mol Ther 15, 1663-1669 (2007).
19. Cristofaro, P. & Opal, S. M. Role of Toll-like receptors in infection and immunity: clinical implications. Drugs 66, 15-29 (2006).
20. Akira, S. & Takeda, K. Functions of toll-like receptors: lessons from KO mice. C R Biol 327, 581-589 (2004).
21. Forsbach, A. et al. Identification of RNA Sequence Motifs Stimulating Sequence-Specific TLR8—Dependent Immune Responses. J Immunol 180, 3729-3738 (2008).
22. Sioud, M. Single-stranded small interfering RNA are more immunostimulatory than their double-stranded counterparts—a central role for 2′-hydroxyl uridines in immune responses. Eur J Immunol 36, 1222-1230 (2006).
23. Ghosh, T. K. et al. Toll-like receptor (TLR) 2-9 agonists-induced cytokines and chemokines: I. Comparison with T cell receptor-induced responses. Cell Immunol 243, 48-57 (2006).
24. Putnam, D. Polymers for gene delivery across length scales. Nat Mater 5, 439-451 (2006).
25. Ewert, K. et al. Cationic lipid-DNA complexes for gene therapy: understanding the relationship between complex structure and gene delivery pathways at the molecular level. Curr Med Chem 11, 133-149 (2004).
26. Akinc, A. et al. A combinatorial library of lipid-like materials for delivery of RNAi therapeutics. Nat Biotech ((submitted)).
27. Nguyen, D. N. et al. A novel high-throughput cell-based method for integrated quantification of type I interferons and in vitro screening of immunostimulatory RNA drug delivery. Biotechnol Bioeng 103, 664-675 (2009).
28. Nguyen, D. N. et al. Drug delivery-mediated control of RNA immunostimulation. Mol Ther 17, 1555-1562 (2009).
29. Nguyen, D. N. et al. A novel high-throughput cell-based method for integrated quantification of type I interferons and in vitro screening of immunostimulatory RNA drug delivery. Biotechnol Bioeng (2009).

Each of the references, patents, and patent applications cited herein is incorporated here in by reference.

### Other Embodiments

The foregoing has been a description of certain non-limiting preferred embodiments of the invention. Those of ordinary skill in the art will appreciate that various changes and modifications to this description may be made without departing from the spirit or scope of the present invention, as defined in the following claims.

1. A method of modulating an immune response in a subject, the method comprising administering to a subject an amount of a nanoparticle sufficient to modulate an immune response in a subject, wherein the nanoparticle comprises a polynucleotide and a lipidoid of formula: 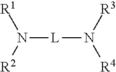
wherein L is a substituted or unsubstituted alkylene group having between one and six carbon atoms, inclusive;
R¹, R², R³, and R⁴ are independently selected from the group consisting of hydrogen, C₁-C₆ alkyl, C₃-C₇ cycloalkyl, —(CH₂)ₙOR^{A}, —(CH₂)ₙN(R^{A})₂, Y, —(CH₂)ₙN(Y)₂, —(CH₂)ₙNR^{A}Y; wherein each occurrence of R^{A} is independently a hydrogen; a protecting group; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted aryl; substituted or unsubstituted heteroaryl; alkoxy; aryloxy; alkylthio; arylthio; amino; alkylamino; dialkylamino; heteroaryloxy; or heteroarylthio moiety; with the proviso that at least one of R¹, R², R³, and R⁴ must equal Y, —(CH₂)ₙN(Y)₂, or —(CH₂)ₙNR^{A}Y;
R¹ and R² may be taken together with the intervening atoms to form a cyclic structure;
R³ and R⁴ may be taken together with the intervening atoms to form a cyclic structure;
each occurrence of Y is independently selected from the group consisting of —CH₂CH₂CO₂R⁸ or —CH₂CH₂CONHR⁸; wherein each occurrence of R⁸ is independently an alkyl chain of 6-30 carbon atoms, inclusive;
each occurrence of n is independently an integer between one and six, inclusive;
and salts thereof.

2. The method of claim 1, wherein at least one of R¹, R², R³, and R⁴ is hydrogen.

3-6. (canceled)

7. The method of claim 1, wherein R¹, R², R³, and R⁴ are not the same.

8. The method of claim 1, wherein at least two of R¹, R², R³, and R⁴ are the same.

9. The method of claim 1, wherein at least three of R¹, R², R³, and R⁴ are the same.

10. The method of claim 1, wherein the lipidoid is of formula: 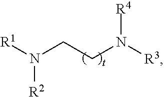
wherein t is 1 or 2.

11. The method of claim 10 wherein the lipidoid is of formula:

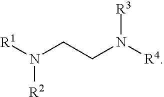

12-28. (canceled)

29. The method of claim 10 wherein the lipidoid is of formula:

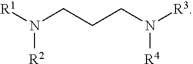

30. (canceled)

31. The method of claim 29, wherein the lipidoid is selected from the group consisting of: 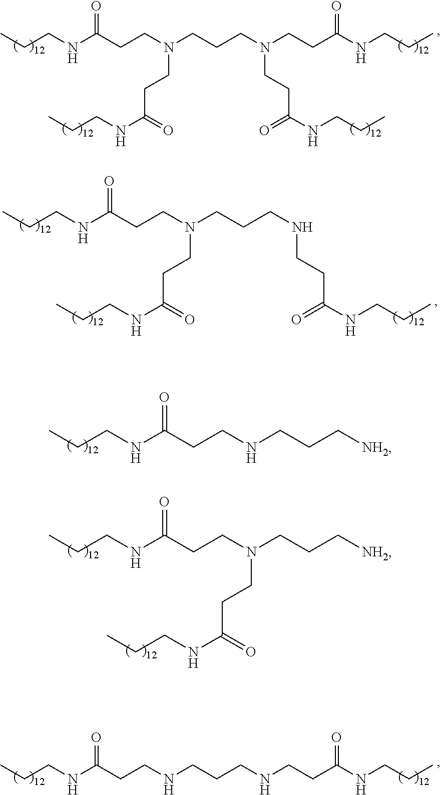
and combinations thereof.

32. The method of claim 29, wherein the lipidoid is selected from the group consisting of: 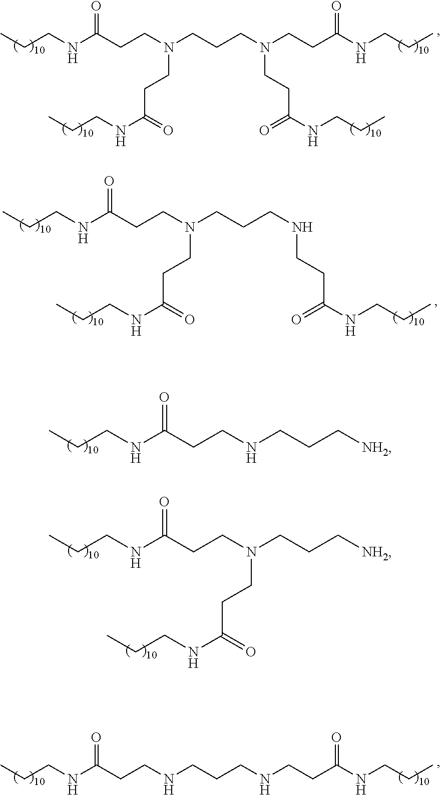
and combinations thereof.

33. The method of claim 29, wherein the lipidoid is selected from the group consisting of: 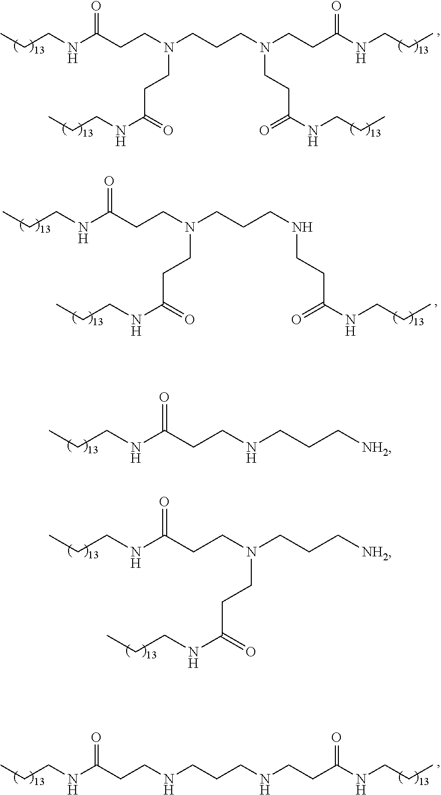
and combinations thereof.

34-59. (canceled)

60. A method of modulating an immune response, the method comprising administering to a subject an amount of a nanoparticle sufficient to modulate an immune response in a subject, wherein the nanoparticle comprises a polynucleotide and a lipidoid of formula: 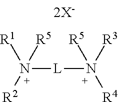
wherein L is a substituted or unsubstituted alkylene group having between one and six carbon atoms, inclusive;
R¹, R², R³, and R⁴ are independently selected from the group consisting of hydrogen, C₁-C₆ alkyl, C₃-C₇ cycloalkyl, —(CH₂)ₙOR^{A}, —(CH₂)ₙN(R^{A})₂, Y, —(CH₂)ₙN(Y)₂, —(CH₂)ₙNR^{A}Y; wherein each occurrence of R^{A} is independently a hydrogen; a protecting group; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted aryl; substituted or unsubstituted heteroaryl; alkoxy; aryloxy; alkylthio; arylthio; amino; alkylamino; dialkylamino; heteroaryloxy; or heteroarylthio moiety; with the proviso that at least one of R¹, R², R³, and R⁴ must equal Y, —(CH₂)ₙN(Y)₂, or —(CH₂)ₙNR^{A}Y;
R¹ and R² may be taken together with the intervening atoms to form a cyclic structure;
R³ and R⁴ may be taken together with the intervening atoms to form a cyclic structure;
each occurrence of Y is independently selected from the group consisting of —CH₂CH₂CO₂R⁸ or —CH₂CH₂CONHR⁸; wherein each occurrence of R⁸ is independently an alkyl chain of 6-30 carbon atoms, inclusive;
each occurrence of n is independently an integer between one and six, inclusive;
R⁵ is hydrogen or C₁-C₆ aliphatic;
X⁻ is an anion.

61-83. (canceled)

84. The method of claim 1, wherein each instance of Y is independently selected from the group consisting of:

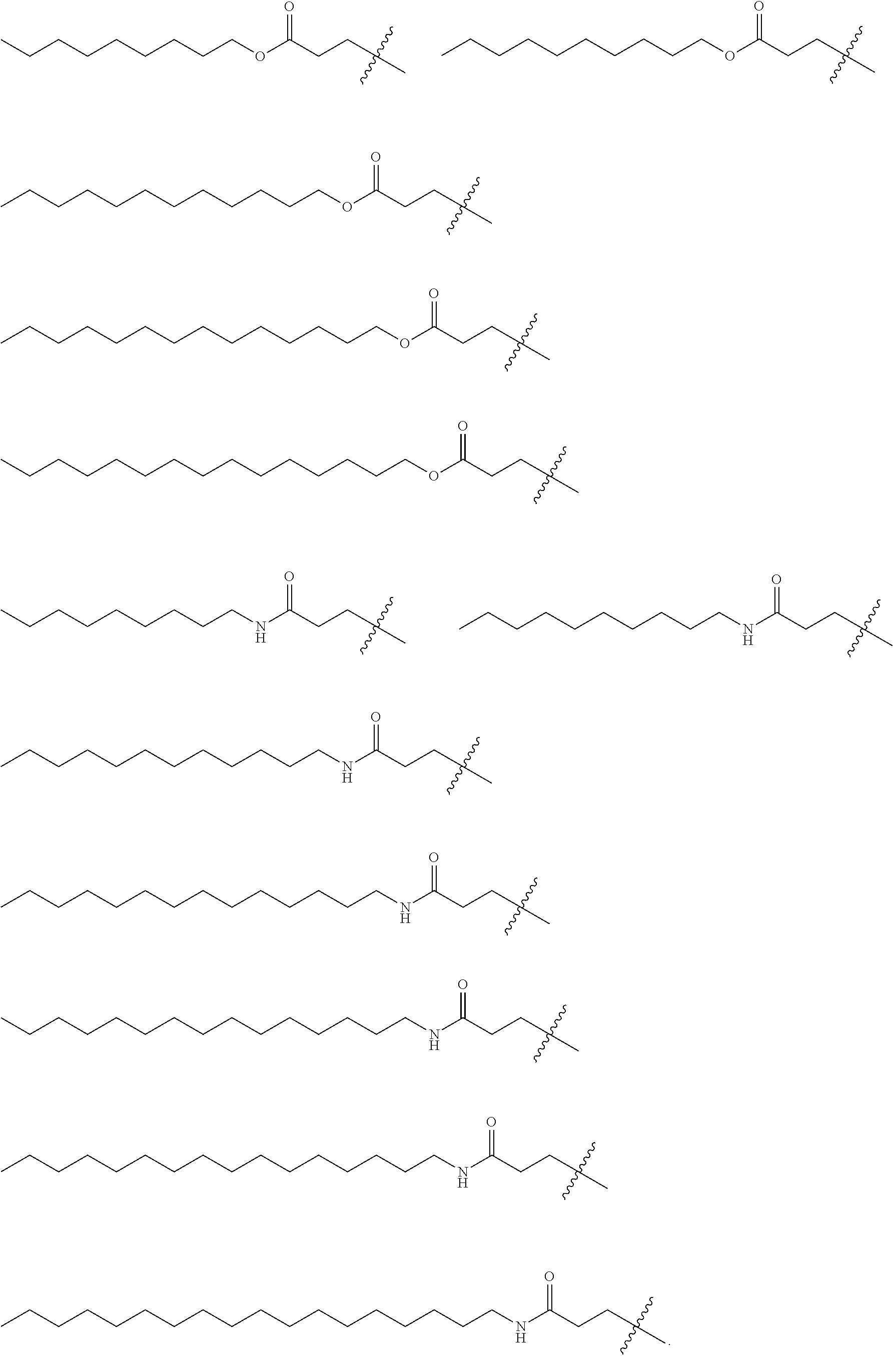

85. (canceled)

86. The method of claim 1, wherein each instance of Y is the same and is selected from the group consisting of:

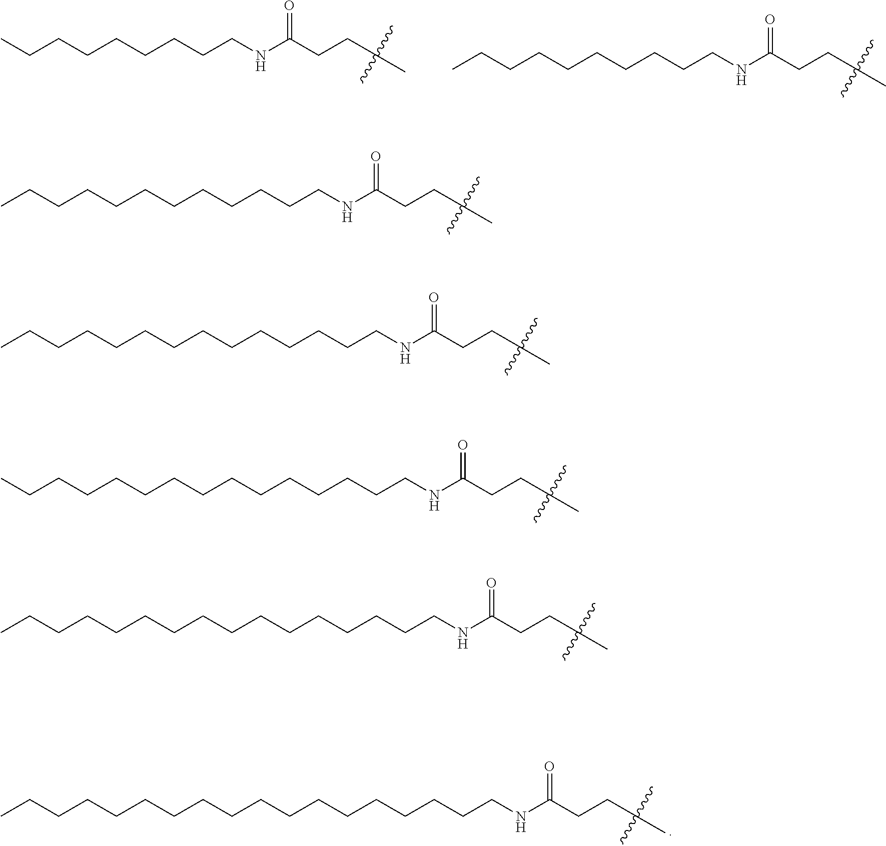

87-98. (canceled)

99. A method of modulating an immune response, the method comprising administering to a subject an amount of a nanoparticle sufficient to modulate an immune response in a subject, wherein the nanoparticle comprises a polynucleotide and a lipidoid of formula: 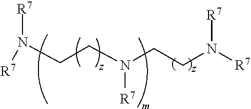
wherein
each occurrence of z is an integer between 1 and 10, inclusive;
m is an integer between 0 and 10, inclusive;
each occurrence of R₇ is hydrogen or Y;
each occurrence of Y is independently selected from the group consisting of —CH₂CH₂CO₂R⁸ or —CH₂CH₂CONHR⁸; wherein each occurrence of R⁸ is independently an alkyl chain of 6-30 carbon atoms, inclusive;
and salts thereof.

100-106. (canceled)

107. The method of claim 1, wherein the polynucleotide is RNA.

108-111. (canceled)

112. The method of claim 1, wherein the polynucleotide is DNA.

113. The method of claim 112, wherein the DNA is CpG DNA.

114. The method of claim 1, wherein the nanoparticle further comprises a polymer.

115-165. (canceled)

166. A pharmaceutical composition for modulating an immune response in a subject comprising an is RNA and a lipidoid of formula: 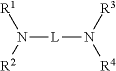
wherein L is a substituted or unsubstituted alkylene group having between one and six carbon atoms, inclusive;
R¹, R², R³, and R⁴ are independently selected from the group consisting of hydrogen, C₁-C₆ alkyl, C₃-C₇ cycloalkyl, —(CH₂)ₙOR^{A}, —(CH₂)ₙN(R^{A})₂, Y, —(CH₂)ₙN(Y)₂, —(CH₂)ₙNR^{A}Y; wherein each occurrence of R^{A} is independently a hydrogen; a protecting group; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted aryl; substituted or unsubstituted heteroaryl; alkoxy; aryloxy; alkylthio; arylthio; amino; alkylamino; dialkylamino; heteroaryloxy; or heteroarylthio moiety; with the proviso that at least one of R¹, R², R³, and R⁴ must equal Y, —(CH₂)ₙN(Y)₂, or —(CH₂)ₙNR^{A}Y;
R¹ and R² may be taken together with the intervening atoms to form a cyclic structure;
R³ and R⁴ may be taken together with the intervening atoms to form a cyclic structure;
each occurrence of Y is independently selected from the group consisting of —CH₂CH₂CO₂R⁸ or —CH₂CH₂CONHR⁸; wherein each occurrence of R⁸ is independently an alkyl chain of 6-30 carbon atoms, inclusive;
each occurrence of n is independently an integer between one and six, inclusive;
and salts thereof.

167-170. (canceled)
